SEQUENCE LISTING (4 sequences)

SEQ ID NO: 1 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 2 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 3 - protein, 448 aa, Homo sapiens
EVRLLESGGGLVQPGGSLRLSCAASGFTFRPRAMAWVRQAPGKGLEWVSSISAQGASAYYADSVKGRFTISRDNSKNTLYLQMNSLRAEDTAVYYCARDLSVSGFGPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 217 aa, Homo sapiens
SYELTQPPSSSGTPGQRVTISCSGSSSNIGRHTVSWYQQVPGTAPKLLIYSDDHRPSGVPDRFSASKSGTSASLTITGLQSEDEADYYCAAWDDSLNGPWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2015/0224191 A1
(43) Pub. Date: Aug. 13, 2015

(54) Combination Treatment with VEGF-C Antagonists

(71) Applicant: Vegenics PTY Limited, South Yarra (AU)

(72) Inventor: Megan E. Baldwin, Brighton East (AU)

(21) Appl. No.: 14/697,175

(22) Filed: Apr. 27, 2015

Related U.S. Application Data
(63) Continuation of application No. 14/013,203, filed on Aug. 29, 2013, now abandoned.
Application No. 14/013,203 is a continuation of application No. 13/501,600, filed on Apr. 12, 2012, now abandoned.
(60) Provisional application No. 61/324,608, filed on Apr. 15, 2010.

(51) Int. Cl. A61K 39/395; A61K 31/337; A61K 31/513; A61K 45/06

(52) U.S. Cl. CPC A61K 39/3955; A61K 45/06; A61K 31/337; A61K 31/513; A61K 2039/507

(57) ABSTRACT

The invention relates to a method and kit for treating cancer in a human subject, the method comprising administering to the subject in combination therapeutically effective amounts of a VEGF-C antagonist and an anti-neoplastic composition, and the kit comprising a VEGF-C antagonist for administering to the subject in combination with an anti-neoplastic composition. The invention further relates to methods for: increasing the duration of survival of, increasing the progression-free survival of, increasing the duration of response of, or treating, a subject or a group of human subjects susceptible to or diagnosed as having a cancer; or treating a human subject or a group of human subjects having metastatic colorectal cancer, prostate cancer, pancreatic cancer or glioblastoma, the methods comprising administering to the subject or subjects in the group in combination effective amounts of a VEGF-C antagonist and an anti-neoplastic composition.

### FIELD

The invention relates to treatment of cancer, comprising administering in combination effective amounts of a VEGF-C antagonist and an anti-neoplastic composition.

### BACKGROUND

Cancer remains one of the most deadly threats to human health. In 2009, cancer was estimated to affect nearly 1.5 million new subjects in the U.S. and was the second leading cause of death after heart disease, accounting for approximately 1 in 4 deaths. It has been predicted that cancer may surpass cardiovascular diseases as the number one cause of death by 2010. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult. Furthermore, cancers can arise from almost any tissue in the body through malignant transformation of one or a few normal cells within the tissue, and each type of cancer with particular tissue origin differs from the others.

Current methods of cancer treatment are relatively non-selective. Surgery removes the diseased tissue; radiotherapy shrinks solid tumors; and chemotherapy kills rapidly dividing cells. Chemotherapy, in particular, results in numerous side effects, in some cases so severe as to limit the dosage that can be given and thus preclude the use of potentially effective drugs. Moreover, cancers often develop resistance to chemotherapeutic drugs.

Thus, there is an urgent need for specific and more effective cancer therapies.

### SUMMARY OF THE INVENTION

A first aspect provides a method of treating cancer in a subject, comprising administering to the subject in combination therapeutically effective amounts of a VEGF-C antagonist and an anti-neoplastic composition.

The method of the first aspect may be presented in alternative forms, for example in European form (“agent for use”) or second medical use (Swiss) form (“use of an agent in the manufacture of a medicament”).

A second aspect provides an article of manufacture comprising a VEGF-C antagonist. The article of manufacture may comprise a kit. The article of manufacture may comprise a container containing the VEGF-C antagonist, and a package insert instructing the user of the VEGF-C antagonist to administer to a subject with cancer the VEGF-C antagonist in combination with an anti-neoplastic composition.

In one embodiment of the second aspect, the article of manufacture comprises a kit comprising the VEGF-C antagonist when used for treating cancer in a human subject, wherein the VEGF-C antagonist is for administering to the subject in combination with an anti-neoplastic composition.

One form designates either suitability for or restriction to a specific use and is indicated by the word “for”. Another form is restricted to a specific use only and is indicated by the words “when used for”.

In an embodiment of the first or second aspect, the VEGF-C antagonist is a VEGF-C antibody.

In an embodiment of the first or second aspect, the subject is human.

In an embodiment of the first or second aspect, the cancer comprises a solid and/or vascularised tumor. The solid tumor may be selected from a sarcoma, a carcinoma, a lymphoma, a melanoma and a blastoma.

In an embodiment of the first or second aspect, the cancer is primary. Therefore, treatment may prevent or ameliorate metastasis and may be in respect of stage I or stage II cancer. In another embodiment of the first or second aspect, the cancer is metastatic and treatment may be in respect of stage III or stage IV cancer.

In one embodiment of the first or second aspect, the anti-neoplastic composition comprises a standard of care for the cancer to be treated.

In an embodiment of the first or second aspect, the cancer is selected from the group consisting of lung and bronchial cancers, colorectal cancers, prostate cancers, pancreatic cancers, liver cancers, esophageal cancers, urinary and bladder cancers, non-Hodgkin lymphomas, kidney and renal cancers, breast cancers, ovarian cancers and brain cancers (e.g. glioblastomas).

In an embodiment of the first or second aspect, the anti-neoplastic composition comprises a chemotherapeutic agent. For example, suitable chemotherapeutic agents include docetaxel, 5-fluorouracil (5-FU), temozolomide (TMZ), gemcitabine, oxaliplatin, paclitaxel, carboplatin and irinotecan.

In another embodiment of the first or second aspect, the anti-neoplastic composition comprises an anti-angiogenic agent. Preferably, the anti-angiogenic agent is an anti-angiogenic antibody. The anti-angiogenic antibody may be a VEGF-A antibody. The VEGF-A antibody may be Bevacizumab.

In a further embodiment of the first or second aspect, the anti-neoplastic composition comprises a chemotherapeutic agent and an anti-angiogenic agent. In one embodiment, the chemotherapeutic agent is selected from the group consisting of docetaxel, 5-fluorouracil (5-FU), temozolomide (TMZ), and gemcitabine, and the anti-angiogenic agent is an antibody such as, for example, Bevacizumab.

In a further embodiment, of the first or second aspect, the cancer comprises a solid and/or vascularised tumor which has become resistant to an anti-angiogenic VEGF-A antagonist, in which the subject receives a combination therapy comprising a VEGF-C antagonist, preferably a VEGF-C antibody, and an anti-neoplastic composition comprising a chemotherapeutic agent and a VEGF-A antagonist, which may be the same or different to the VEGF-A antagonist to which the subject has become resistant. In one embodiment, the VEGF-A antagonist to which the subject has become resistant to bevacizumab.

### BRIEF DESCRIPTION OF THE DRAWINGS

FIG. 1 provides an amino acid sequence of human VEGF-C(SEQ ID NO: 1). The VHD of human VEGF-C is underlined.

FIG. 2 provides an amino acid sequence of human VEGF-A (SEQ ID NO: 2).

FIG. 3 provides an amino acid sequence for the heavy chain of the VEGF-C antibody VGX-100 (SEQ ID NO: 3). The heavy chain variable (VH) region is underlined.

FIG. 4 provides an amino acid sequence for the light chain of the antibody of FIG. 3 (SEQ ID NO: 4). The light chain variable (VL) region is underlined.

FIG. 5 depicts the response (tumor volume (mm³)) over time (days) to single treatment of PC-3 human prostate tumors (xenograft) according to Example 2. A VEGF-C antibody (VGX-100) was tested at 10 (*), 20 (♦) and 40 (Δ) mg/kg and compared to a VEGF-A antibody (bevacizumab, Avastin) at 10 mg/kg ( [symbol] ). Antibodies were administered twice per week by intraperitoneal injection. The negative control is indicated by the uppermost line (♦). VGX-100 at 10 and 20 mg/kg were indistinguishable, improved over the negative control, but not as efficacious as VGX-100 at 40 mg/kg or bevacizumab at 10 mg/kg. VGX-100 at 40 mg/kg displayed efficacy equivalent to bevacizumab at 10 mg/kg.

FIG. 6 depicts the mean cancer tumor size (mg) over time (days post tumor implant up to 160) in a PC-3 human prostate tumor xenograft model in nude mice in a study according to Example 3. Mice were treated with a VEGF-C antibody (VGX-100) as a single agent therapy or in a combination therapy with a VEGF-A antibody (bevacizumab, Avastin) and/or a chemotherapeutic agent (docetaxel). VGX-100 (40 mg/kg) and bevacizumab (10 mg/kg) were administered by intraperitoneal injection twice weekly. Docetaxel (10 mg/kg) was administered by intravenous injection on days 7, 14 and 21. At 160 days post implant, the lowermost line is the triple therapy of VGX-100+bevacizumab+deocetaxel, and in increasing order VGX-100+docetaxel, bevacizumab+docetaxol, docetaxel. The remaining treatments are indistinguishable at 160 days. At 60 days, however, the negative isotype control is uppermost, and in decreasing order VGX-100, bevacizumab and VGX-100+bevacizumab.

FIG. 7 depicts the tumor burden in the various treatment groups of FIG. 6.

FIG. 8 depicts the survival rate of the mice of FIG. 6. At 160 days post implant, the uppermost line is the triple therapy of VGX-100+bevacizumab+deocetaxel, and in decreasing order VGX-100+docetaxel, bevacizumab+docetaxol, docetaxel, negative isotype control, bevacizumab, VGX-100+bevacizumab, and VGX-100.

FIG. 9 depicts the response (mean tumor burden (mg)) over time (days post tumor implant, up to 70 days) to single or combination treatment of PC-3 human prostate tumors in nude mice in a second study according to Example 3. A VEGF-C antibody (VGX-100) was tested as a single agent therapy or in a combination therapy with a VEGF-A antibody (bevacizumab, Avastin) and/or a chemotherapeutic agent (docetaxel). VGX-100 (40 mg/kg) and bevacizumab (10 mg/kg) were administered by intraperitoneal injection twice weekly. Docetaxel (10 mg/kg) was administered by intravenous injection on days 7, 14 and 21. At 49 days post implant, the uppermost line is the negative isotype control (◯) and in decreasing order bevacizumab ( [symbol] ), VGX-100+bevacizumab (⊙), VGX-100 (□), docetaxel ( [symbol] ), VGX-100+docetaxel (▪), bevacizumab+docetaxel (Δ), and the triple therapy of VGX-100+bevacizumab+docetaxel (*). The triple therapy performed better than any of the dual therapies.

FIG. 10 depicts the percentage of mice of Example 4 with prostate tumors up to 7 weeks after intra-prostatic tumor inoculation. The VEGF-C antibody VGX-100 in combination with docetaxel (0.5 mg/kg; □) delayed the appearance of tumors in this model. VGX-100 (◯); negative isotype control (); docetaxel (▪); docetaxel+VGX-100 (□); docetaxel (▴); docetaxel+VGX-100 (Δ); PSA control ( [symbol] ).

FIG. 11 depicts the response (tumor volume (mm³)) over time (days) to single treatment of U87MG glioblastoma tumors (xenograft) according to Example 6. A VEGF-C antibody (VGX-100) was tested at 10 (x), 20 (x) and 40 (▴) mg/kg and compared to a VEGF-A antibody (bevacizumab, Avastin) at 10 mg/kg (▪). Antibodies were administered twice per week by intraperitoneal injection. The negative control is indicated by the uppermost line. VGX-100 at 10 and 20 mg/kg were indistinguishable, improved over the negative control, but not as efficacious as VGX-100 at 40 mg/kg or bevacizumab at 10 mg/kg. VGX-100 at 40 mg/kg displayed efficacy relevant to the negative control.

FIG. 12 illustrates the effect (mean tumor burden (mg)) of the VEGF-C antibody VGX-100 as a single therapy or in combination with bevacizumab on growth of U87MG human glioblastoma tumors in nude mice according to Example 7. Bevacizumab (10 mg/kg) and VGX-100 (40 mg/kg) were administered by intraperitoneal injection twice weekly. At the final time-point, the negative isotype control is the uppermost line () and in decreasing order VGX-100 alone (▪), bevacizumab alone ( [symbol] ), and VGX-100+bevacizumab ( [symbol] ).

FIG. 13 depicts the effect (mean tumor burden (mm³)) over time (days post tumor implant) of the VEGF-C antibody VGX-100 as a single-agent or in combination with bevacizumab on growth of KP4 human pancreatic tumors in nude mice according to Example 8. VGX-100 and bevacizumab were administered by intraperitoneal injection twice weekly. VGX-100 was administered at 20 or 40 mg/kg for single therapy and at 40/mg/kg for combination therapy. Bevacizumab was administered at 10 mg/kg for single and combination therapy. At 30 days post implant, the negative isotype control is the uppermost line, and in descending order VGX-100 at 20 mg/kg, VGX-100 at 40 mg/kg, bevacizumab, and VGX-100 plus bevacizumab is the lowermost line.

FIG. 14 shows the binding of VGX-100 to both VEGF-C and VEGF-D monomer and dimer in an ELISA assay as measured using BiaCore at an absorbance wavelength of 450 nm.

FIG. 15 shows the effect of VGX-100 on the binding of VEGF-C to (a) VEGFR-2 and (b) VEGFR-3 using a Ba/F3 bioassay (Stacker et al. 1999 J Biol Chem 274: 34884-34892; Achen et al. 2000 Eur J Biochem 267: 2505-2515). The binding of VEGF-C to the extracellular domain of VEGFR-2 or VEGFR-3 was measured using BA/F3-VEGFR-2 or -3/EpoR cells. The response to ligands and VGX-100 was measured by [³H]-thymidine incorporation following exposure for 48 hrs.

FIG. 16 shows the results of a HUVEC proliferation assay in which VGX-100 was used to inhibit the biological activity of VEGF-C.

FIG. 17 shows the effect (mean tumor burden (mg)) over time (days post tumor implant) of the VEGF-C antibody VGX-100 as a single-agent or in combination with bevacizumab (Avastin) and/or 5-FU on growth of HCT-116 human colorectal tumors in nude mice according to Example 10. Isotype control (◯); bevacizumab alone ( [symbol] ); VGX-100 alone (□); 5-FU alone (⋄); bevacizumab+5-FU (Δ); VGX-100+5-FU (▪); VGX-100+bevacizumab (⊙); VGX-100+bevacizumab+5-FU (*).

FIG. 18 shows the effect (mean tumor burden (mg)) over time (days post tumor implant) of the VEGF-C antibody VGX-100 as a single-agent or in combination with bevacizumab and/or docetaxel on growth of H292 human lung tumors in nude mice according to Example 11. From the uppermost line progressing downwards: VGX-100 alone ( [symbol] ); Isotype control (); docetaxel alone ( [symbol] ); bevacizumab alone (); VGX-100+bevacizumab ( [symbol] ); bevacizumab+docetaxel ( [symbol] ); VGX-100+bevacizumab+docetaxel ( [symbol] ).

FIG. 19 shows the effect (mean tumor burden (mg)) over time (days post tumor implant) of the VEGF-C antibody VGX-100 in combination with bevacizumab and docetaxel on growth of OVCAR-8 human ovarian tumors in nude mice according to Example 12. From the uppermost line progressing downwards: Isotype control (); docetaxel alone ( [symbol] ); bevacizumab+docetaxel ( [symbol] ); VGX-100+bevacizumab+docetaxel ( [symbol] ).

FIG. 20 shows the effect (mean tumor burden (mm³)) over time (days post tumor implant) of the VEGF-C antibody VGX-100 as a single agent on growth of PC-3-GFP human prostate tumors in an orthotopic MetaMouse® model according to Example 13. Isotype control (); VGX-100 alone ( [symbol] ).

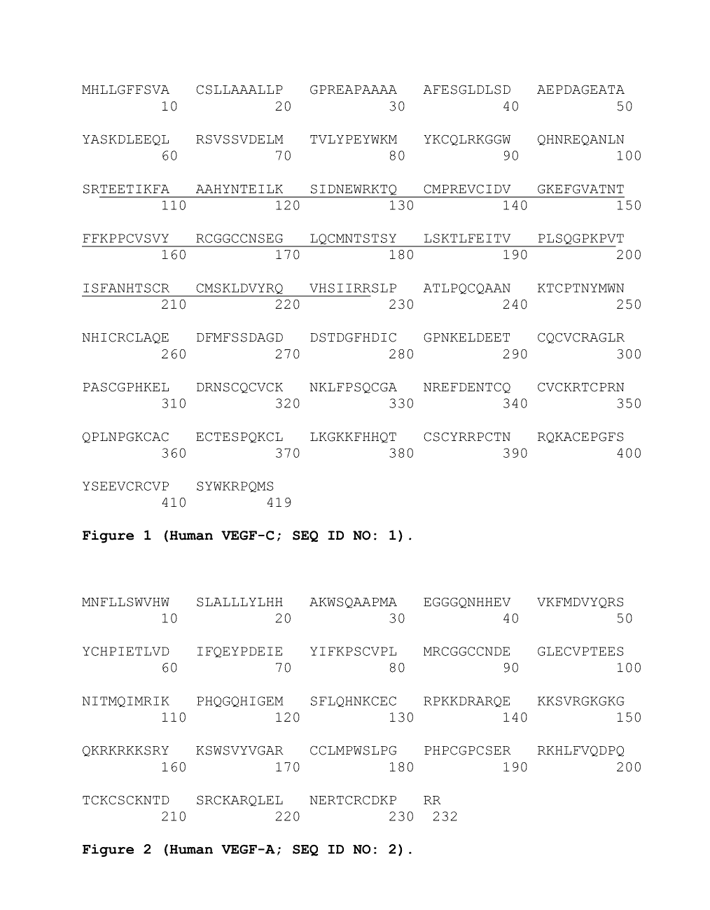

### DETAILED DESCRIPTION

### Definitions

A “disease” or “disorder” is any condition or phenotype that would benefit from treatment with a substance, composition or method of the invention. This includes chronic and acute disorders or diseases including those pathological conditions which predispose the mammal to the disorder in question. Non-limiting examples of disorders to be treated herein include benign and malignant tumors; leukemias and lymphoid malignancies; neuronal, glial, astrocytal, hypothalamic and other glandular, macrophagal, epithelial, stromal and blastocoelic disorders; inflammatory, immunologic and other angiogenesis-related disorders; and cancer.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a cancer. In one embodiment, the cell proliferative disorder is angiogenesis.

The term “tumor” refers to all neoplastic cell growth and proliferation, whether malignant or benign, and all-pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in a mammal that is typically characterized by unregulated cell growth. Examples of cancer include but are not limited to, carcinoma, lymphoma, blastoma, sarcoma, melanoma and leukemia or lymphoid malignancies. More particular examples of such cancers include adrenocortical carcinoma, adenocarcinoma of the lung, AIDS-related cancers and lymphomas, anal cancer, astrocytoma, B-cell lymphomas (including low grade/follicular non-Hodgkin's lymphoma (NHL), small lymphocytic NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma and Waldenstrom's Macroglobulinemia), bladder cancer, breast cancer (including male breast cancer), bronchial cancer, cancer of the intrahepatic bile duct, carcinoid tumors, cervical cancer, chronic lymphocytic leukemia, chronic myeloblastic leukemia, chronic myelogenous leukemia, chronic myeloproliferative disorders, clear cell sarcoma, colon cancer, colorectal cancer, cutaneous T-cell lymphoma, endometrial cancer, ependymoma, esophageal cancer, Ewing's sarcomas, gall bladder cancer, gastric or stomach cancer (including gastrointestinal cancer, germ cell tumors, gestational trophoblastic tumors, glioma or brain cancers (including glioblastoma), hairy cell leukemia, head and neck cancers, hepatocellular carcinoma, hypopharyngeal cancer, islet cell carcinoma, intraoccular melanoma, Kaposi's sarcoma, kidney cancer, laryngeal cancer, acute lymphoblastic leukemia, acute myeloid leukemia, hairy cell leukemia, lip and oral cavity cancer, liver cancer, lung cancer (including non-small cell and small-cell lung cancers), cutaneous T-cell lymphoma, Hodgkin's lymphoma, non-Hodgkin's lymphoma, Waldenstrom's macroglobulinemia, malignant fibrous histiocytoma, malignant mesothelioma, medulloblastoma, Merkel cell carcinoma, malignant mesothelioma, squamous neck cancer with occult primary, multiple endocrine neoplasia syndrome, multiple myeloma, mycosis fungoides, myelodysplastic syndromes, chronic myelogenous leukemia, acute myeloid leukemia, myeloproliferative disorders, nasal cavity and paranasal sinus cancer, nasopharyngeal cancer, neuroblastoma, oropharyngeal cancer, osteosarcoma, ovarian cancer, ovarian epithelial cancer, ovarian germ cell tumor, pancreatic cancer, islet cell pancreatic cancer, parathyroid cancer, penile cancer, peritoneal cancer, pheochromocytoma, pituitary tumor, plasma cell neoplasms, pleuropulmonary blastoma, post-transplant lymphoproliferative disorder, primary central nervous system lymphoma, primary liver cancer, primitive neuroectodermal tumors, prostate cancer, rectal cancer, renal cell (kidney) cancer, transitional cell cancer of the renal pelvis and ureter cancer, retinoblastoma, rhabdomyosarcoma, salivary gland cancer, soft tissue sarcoma, Sezary syndrome, skin cancer, skin melanoma, Merkel cell skin carcinoma, small intestine cancer, squamous cell cancer, squamous carcinoma of the lung, testicular cancer, thymoma, thymic carcinoma, thyroid cancer, gestational trophoblastic tumor, carcinoma of unknown primary site, urethral cancer, uterine cancer, uterine sarcoma, vaginal cancer, vulvar cancer, Wilms' tumor, as well as abnormal vascular proliferation associated with phakomatoses, edema (such as that associated with brain tumors), and Meigs' syndrome.

The term “anti-neoplastic composition” refers to a composition useful in treating cancer. Preferably, the anti-neoplastic composition comprises at least one active therapeutic agent capable of inhibiting or preventing tumor growth or function, and/or causing destruction of tumor cells. Examples of suitable therapeutic agents (anti-cancer agents) include, but are not limited to, chemotherapeutic agents, agents used in radiation therapy (e.g. radioactive isotopes), cytotoxic agents, growth inhibitory agents, toxins, anti-angiogenic agents, anti-lymphangiogenic agents, apoptotic agents, anti-tubilin agents and other agents to treat cancer such HER2-antibodies, CD-20 antibodies, an epidermal growth factor receptor (EGFR) antagonist (e.g. a tyrosine kinase inhibitor), a HER1/EGFR inhibitor (e.g. erlotinib (Tarceva™), platelet derived growth factor inhibitors (e.g. Gleevac™ (Imatinib Mesylate)), a COX-2 inhibitor (e.g. celecoxib), cytokines, interferons, antagonistic agents (e.g. neutralising antibodies) that bind to, for example, one or more of the following, ErbB2, ErbB3, ErbB4, PDGFR-beta, BIyS, APRIL, BCMA, VEGF-A, VEGF-C, VEGF-D, VEGF receptors (e.g. VEGFR-1, VEGFR-2, VEGFR-3, NP-1 and NP-2), TRAIL/Apo2 and other bioactive and organic chemical agents. Combinations thereof are also included in the invention.

The term “cytotoxic agent” refers to a substance that inhibits or prevents the function of cells and/or causes destruction of cells. The term is intended to include: Radioactive isotopes (e.g., At²¹¹, I¹³¹, I¹²⁵, Y⁹⁰, R¹⁸⁶, Re¹⁸⁸, Sm¹⁵³, Bi²¹², P³² and radioactive isotopes of Lu); chemotherapeutic agents such as methotrexate, adriamicin, vinca alkaloids (vincristine, vinblastine, etoposide), doxorubicin, melphalan, mitomycin C, chlorambucil, daunorubicin or other intercalating agents; enzymes and fragments thereof such as nucleolytic enzymes; antibiotics; toxins such as small molecule toxins or enzymatically active toxins of bacterial, fungal, plant or animal origin, including fragments and/or variants thereof; the various antitumor or anticancer agents disclosed below. Other cytotoxic agents are described below. A tumoricidal agent causes destruction of tumor cells.

The term “chemotherapeutic agent” refers to a chemical compound useful in the treatment of cancer. Chemotherapeutic agents are broadly categorized into classes, for example, based on common structural motifs, mechanism of actions and/or the organisms from which they are derived. For example, chemotherapeutic agents may be classified as alkylating agents, anti-metabolites, alkaloids, terpenoids, topoisomerase inhibitors, antibiotics, androgens and anti-hormonals. It will be apparent that many chemotherapeutic agents fall into one or more class or that they are variously referred to by the medical profession as belonging to different classes.

Alkylating Chemotherapeutic Agents:

Alkylating agents are so named because of their ability to alkylate many nucleophilic functional groups under conditions present in cells. Alkylating agents are sometimes variously referred to as classical alkylating, alkylating-like and non-classical alkylating agents. In contrast to the alkylating-like and non-classical agents, the classical alkylating agents include true alkyl groups and have been known for longer than the other alkylating agents. The classical alkylating agents work by impairing cell function by forming covalent bonds with the amino, carboxyl, sulfhydryl, and phosphate groups in biologically important molecules. Examples of classical alkylating agents include the nitrogen mustards, the nitrosoureas and the alkyl sulfonates. The ethylene imines (aziridines) and methyl melamines are also generally considered as classical, but can be considered non-classical as well. Specific examples of classical alkylating agents include: nitrogen mustards such as chlorambucil, chlornaphazine, cyclophosphamide (e.g. CYTOXAN®), estramustine, ifosfamide, mechlorethamine (mustine HN2), mechlorethamine oxide hydrochloride, melphalan, novembichin, phenesterine, prednimustine, trofosfamide, uracil mustard (uramustine) and mannomustards; nitrosoureas such as carmustine, chlorozotocin, fotemustine, lomustine and nimustine; alkyl sulfonates such as busulfan, improsulfan and piposulfan; ethylene imines (aziridines) and methyl melamines such as altretamine, triethylenemelamine, triethylenephosphoramide (TEPA), triethylenethiophosphoramide (e.g. ThioTEPA) and trimethylol melamine. Alkylating-like agents include platinum-based chemotherapeutic drugs (often referred to as platinum analogues). Although these agents do not have an alkyl group, they nevertheless damage DNA by permanently coordinating to DNA to interfere with DNA repair. Examples of platinum analogues include cisplatin (e.g. Carboquone), carboplatin, nedaplatin, oxiloplatin, oxaliplatin (Eloxatin™) and satraplatin. Some of the agents variously included in the non-classical alkylating category include procarbazine, triethylenethiophosphoramide (e.g. ThioTEPA) and its analogues such as altretamine (which are considered classical alkylating agents by some) and certain tetrazines such as dicarbazine and temozolomide.

Anti-Metabolite Chemotherapeutic Agents:

Anti-metabolites masquerade as purines or pyrimidines—which become the building blocks of DNA. They prevent these substances from becoming incorporated in to DNA during the “S” phase (of the cell cycle), stopping normal development and division. They also affect RNA synthesis. Due to their efficiency, these drugs are the most widely used cytostatics. Examples of anti-metabolites include purine analogues, pyrimidine analogues and antifolates. Examples of purine analogues include azathioprine, fludarabine, mercaptopurine (e.g. 6-mercaptopurine), thiamiprine, thioguanine (e.g. 6-thioguanine), pentostatin and cladribine. Examples of pyrimidine analogues include ancitabine, azacitidine, 6-azauridine, carmofur, dideoxyuridine, doxifluridine, enocitabine, floxuridin, floxuridine, 5-fluorouracil (5-FU) and gemcitabine (GEMZAR®). Examples of antifolates include methotrexate, denopterin, pteropterin, trimetrexate, trimethoprim, pyrimethamine, premetrexed, edatraxate and reltitrexed.

Alkaloid and Terpenoid Chemotherapeutic Agents:

Alkaloids and terpenoids are derived from plants and animals and generally block cell division by preventing microtubule function. The main examples are vinca alkaloids and taxanes. Examples of vinca alkaloids (which are derived from the Madagascar periwinkle and which bind to tubulin preventing assembly of tubulin into microtubules) include vinblastine (VELBAN®), vincristine (ONCOVIN®), vinorelbine (NAVELBINE®) and vindesine (ELDISINE®, FILDESIN®). Taxanes or taxoids are based on taxol (paclitaxel) which is derived from the bark of the Pacific Yew tree. They work by enhancing stability of microtubules thereby preventing separation of chromosomes during anaphase. Examples of taxanes/taxoids include paclitaxel (TAXOL® paclitaxel), abraxane (ABRAXANE™ Cremophor-free, albumin-engineered nanoparticle formulation of paclitaxel) and docetaxel (TAXOTERE® doxetaxel). Terpenoids are a large and diverse class of naturally-occurring organic chemicals found in all classes of living organisms. They are under investigation by numerous groups for anti-tumor and other therapeutic properties. Examples of terpenoids include eleutherobin. Another group of plant-derived chemotherapeutic agents are based on podophyllotoxin. Podophyllotoxin is used to produce other cytostatic drugs including etoposide, epopside phosphate, teniposide and amsacrine.

Topoisomerase Inhibitor Chemotherapeutic Agents:

Topoisomerases are essential enzymes that maintain the topology of DNA. Inhibition of type I or type II topoisomerases interefers with both transcription and replication of DNA by upsetting proper DNA supercoiling. Examples of topoisomerase inhibitors include beta-lapachone, lapachol, betulinic acid, doxorubicin, camptothecin (CPT), topotecan (HYCAMTIN®), CPT-11 (irinotecan, CAMPTOSAR®), acetylcamptothecin, scopolectin, 9-aminocamptothecin, LURTOTECAN® topoisomerase I inhibitor, lamellarin D, topoisomerase inhibitor RFS 2000, podophyllotoxins, derivatives of epipodophyllotoxins (such as amsacrine, etoposide (VP-16), epopside phosphate and teniposide), fluoroquinolones (such as ciprofloxacin, norfloxacin, lomefloxacin and ofloxacin);

Antibiotic Chemotherapeutic Agents:

Many antibiotics are used as chemotherapeutic agents including anthracyclines, enediynes, actinomycins, bleomycins, mithramycin and mitomycins. Most of these have been isolated from natural sources and antibiotics, however, they lack the specificity of conventional antimicrobial antibiotics and thus produce significant toxicity. The general properties of these drugs include interaction with DNA in a variety of different ways including intercalation (squeezing between the base pairs), DNA strand breakage and inhibition with the enzyme topoisomerase II. Examples of anthracycline antibiotics (including the anthraquinones) include daunorubicin (Daunomycin), daunorubicin (liposomal), daunomycin (daunomycin cerubidine), doxorubicin (ADRIAMYCIN® doxorubicin), morpholino-doxorubicin, cyanomorpholino-doxorubicin, 2-pyrrolino-doxorubicin, 4-deoxydoxorubicin (esorubicin), doxorubicin (liposomal), epirubicin, carminomycin, idarubicin, valrubicin, mitoxantrone, detorubicin, rodorubicin (a tetraglycosidic anthracycline), pirarubicin and zorubicin. Examples of enediynes antibiotics include calicheamicin (such as calicheamicin gamma 11 and calicheamicin omega1I (see, e.g., Agnew, Chem. Intl. Ed. Engl., 33: 183-186 (1994)), dynemicin (such as dynemicin A), zinostatin and enediyne chromoproteins (such as esperamicin, neocarzinostatin chromophore and related chromoprotein enediyne antiobiotic chromophores). Other antibiotics include aclacinomycins, actinomycin, azaserine, bleomycins, carabicin, carzinophilin, chromomycins, Actinomycin D (Dactinomycin), 6-diazo-5oxo-L-norleucine, marcellomycin, mitomycins such as mitomycin C, mycophenolic acid, nogalamycin, olivomycins, peplomycin, potfiromycin, puromycin, quelamycin, streptonigrin, streptozocin, tubercidin, ubenimex, CC-1065 (including its adozelesin, carzelesin and bizelesin synthetic analogues), duocarmycin (including the synthetic analogues KW-2189 and CB1-TM1).

Androgen Chemotherapeutic Agents:

Androgens such as calusterone, dromostanolone propionate, epitiostanol and mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; bestrabucil; bisantrene; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; prednisone; phenamet; losoxantrone; 2-ethylhydrazide; PSK® polysaccharide complex; razoxane; rhizoxin; sizofuran; spirogermanium; tenuazonic acid; triaziquone; 2,2′,2″-trichlorotriethylamine; trichothecenes (especially T-2 toxin, verracurin A, roridinA and anguidine); urethan; dacarbazine; mitolactol; pipobroman; gacytosine; mitoxantrone; leucovovin; novantrone; aminopterin; ibandronate; difluoromethylornithine (DMFO); retinoids such as retinoic acid; capecitabine (XELODA®);

Anti-Hormonal Chemotherapeutic Agents:

Anti-hormonal chemotherapeutic agents act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone and FARESTON® toremifene; anti-progesterones; estrogen receptor down-regulators (ERDs); agents that function to suppress or shut down the ovaries or testes, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole.

Other chemotherapeutic agents include the polyketides/macrocyclic lactones such as bryostatin and callystatin; the acetogenins such as bullatacin and bullatacinone; the bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate and ACTONEL® risedronate; nucleoside analogues such as troxacitabine (a 1,3-dioxolane nucleoside cytosine analog) and cytarabine (cytosine arabinoside or Ara-C); antisense oligonucleotides, particularly those that inhibit expression of genes in signaling pathways implicated in abherant cell proliferation, such as, for example, PKC-alpha, Raf, H-Ras and epidermal growth factor receptor (EGF-R); vaccines such as THERATOPE® vaccine and gene therapy vaccines, for example, ALLOVECTIN® vaccine, LEUVECTIN® vaccine and VAXID® vaccine; ABARELIX® rmRH; lapatinib ditosylate (an ErbB-2 and EGFR dual tyrosine kinase small molecule inhibitor also known as GW572016); colchicines; podophyllinic acid; cryptophycins including cryptophycin 1 and cryptophycin 8; dolastatin; eleutherobin; pancratistatin; a sarcodictyin and spongistatin.

Chemotherapeutic agents also include pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional chemotherapeutic agents include the cytotoxic agents useful as antibody drug conjugates, such as, for example, maytansinoids (DMI, for example) and the auristatins MMAE and MMAF.

The term “growth inhibitory agent” refers to a compound or composition which inhibits growth and/or proliferation of a cell. Examples of growth inhibitory agents include agents that block cell cycle progression (at a place other than S phase), such as agents that induce GI arrest and M-phase arrest. Classical M-phase blockers include the vinca alkaloids (e.g. vincristine and vinblastine), taxanes and topoisomerase II inhibitors such as the anthracycline antibiotic doxorubicin ((8S-cis)-10-[(3-amino-2,3,6-trideoxy-a-Llyxo-hexapyranosyl)oxy]-7,8,9,10-tetrahydro-6,8,11-trihydroxy-8-(hydroxyacetyl)-1-methoxy-5,12-naphthacenedione), epirubicin, daunorubicin, etoposide and bleomycin. Those agents that arrest GI also spill over into S-phase arrest, for example, DNA alkylating agents such as tamoxifen, prednisone, dacarbazine, mechlorethamine, cisplatin, methotrexate, 5-fluorouracil and ara-C. Further information can be found in The Molecular Basis of Cancer, Mendelsohn and Israel, eds., Chapter I, entitled “Cell cycle regulation, oncogenes, and anti-neoplastic drugs” by Murakami et al. (WB Saunders: Philadelphia, 1995). The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “cytokine” refers to proteins released by one cell population which act on another cell as intercellular mediators. Examples of such cytokines are lymphokines, monokines, and traditional polypeptide hormones. Included among the cytokines are: growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); epidermal growth factor; hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma colony stimulating factors (CSFs) such as macrophage-CSF (M-CSF); granulocyte-macrophage-CSF (GM-CSF); and granulocyte-CSF (G-CSF); interleukins (Ls) such as IL-1, IL-lalpha, IL-2, IL-3, IL-4, IL-5, IL-6, JL-7, IL-8, IL-9, IL-10, IL-11, IL-12; a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

The term “prodrug” refers to a precursor or derivative form of a pharmaceutically active substance that is less cytotoxic to tumor cells compared to the parent drug and is capable of being enzymatically activated or converted into the more active parent form. The prodrugs relevant to this disclosure include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form include, but are not limited to, those chemotherapeutic agents described above.

The term “angiogenic factor or agent” refers to a growth factor or its receptor which is involved in stimulating the development of blood vessels, e.g., promoting angiogenesis, endothelial cell growth, stability of blood vessels, and/or vasculogenesis, etc. For example, angiogenic factors, include, but are not limited to, VEGF-A and members of the VEGF family and their receptors (VEGF-B, VEGF-C, VEGF-D, VEGFR-1, VEGFR-2 and VEGFR-3); placental growth factor (PIGF); members of the platelet-derived growth factor (PDGF) family and their receptors (especially PDGF-BB, PDGFR-alpha, or PDGFR-beta); members of the fibroblast growth factor (FGF) family and their receptors (acidic (aFGF), basic (bFGF), FGF4, FGF9); TIE ligands and their receptors (angiopoietins, ANGPTI, ANGPT2, TIE1, TIE2); ephrins, Bv8, Delta-like ligand 4 (DLL4), Del-1, BMP9, BMP10, follistatin, granulocyte colony-stimulating factor (G-CSF), granulocyte-macrophage colony-stimulating factor (GM-CSF), hepatocyte growth factor/scatter factor (HGF/SF), interleukin-8 (IL-8), CXCL12, leptin, midkine, neuropilins (NRP1, NRP2), platelet-derived endothelial cell growth factor (PD-ECGF), pleiotrophin (PTN), progranulin, proliferin, transforming growth factor-alpha (TGF-alpha), transforming growth factor-beta (TGF-beta), tumor necrosis factor-alpha (TNF-alpha), Alkl, CXCR4, Notch1, Notct4, Sema3A, Sema3C, Sema3F, Robo4, etc. It would further include factors that promote angiogenesis, such as Perlecan (PLC) also known as basement membrane-specific heparan sulfate proteoglycan core protein (HSPG) or heparan sulfate proteoglycan 2 (HSPG2). It would also include factors that accelerate wound healing, such as growth hormone, insulin-like growth factor-I (IGF-I), vascular IBP-like growth factor (VIGF), epidermal growth factor (EGF), EGF-like domain multiple 7 (EGFL7) and connective tissue growth factor (CTGF) and members of its family. See, e.g., Klagsbrun & D'Amore (1991) Annu. Rev. Physiol. 53:217-39; Streit and Detmar (2003) Oncogene 22:3172-3179; Ferrara & Alitalo (1999) Nature Medicine 5(12):1359-1364; Tonini et al. (2003) Oncogene 22:6549-6556 (e.g., Table I listing known angiogenic factors); Sato (2003) Int. J. Clin. Oncol. 8:200-206.

The term “anti-angiogenic agent” or “angiogenic inhibitor” refers to a small molecular weight substance, a polynucleotide (including, e.g., an inhibitory RNA (RNAi or siRNA)), a polypeptide, an isolated protein, a recombinant protein, an antibody or conjugates or fusion proteins thereof, that inhibits angiogenesis, vasculogenesis, or undesirable vascular permeability, either directly or indirectly. It should be understood that the anti-angiogenic agent includes those agents that bind and block the angiogenic activity of the angiogenic factor (as defined above) or its receptor. For example, an anti-angiogenic agent is an antibody or other antagonist to an angiogenic agent as defined below, e.g., antibodies to VEGF-A or to the VEGF-A receptor (e.g. VEGFR-1 or VEGFR-2), anti-PDGFR inhibitors, small molecules that block VEGF-A receptor signaling (e.g., PTK787/ZK2284, SU6668, SUTENT®/SUI1248 (sunitinib malate), AMG706, or those described in, e.g., WO 2004/113304. Anti-angiogenic agents include, but are not limited to, the following agents: VEGF-A inhibitors such as a VEGF-specific antagonist, EGF inhibitors, EGFR inhibitors, Erbitux® (cetuximab, ImClone Systems, Inc., Branchburg, N.J.), Vectibix® (panitumumab, Amgen, Thousand Oaks, Calif.), TIE2 inhibitors, IGF1R inhibitors, COX-II (cyclooxygenase II) inhibitors, MMP-2 (matrixmetalloproteinase 2) inhibitors, and MMP-9 (matrix-metalloproteinase 9) inhibitors, CP-547,632 (Pfizer Inc., N.Y., USA), Axitinib (Pfizer Inc.; AG-013736), ZD-6474 (AstraZeneca), AEE788 (Novartis), AZD-2171), VEGF Trap (RegeneronJAventis), Vatalanib (also known as PTK-787, ZK-222584: Novartis & Schering A G), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. Other angiogenesis inhibitors include thrombospondin1, thrombospondin2, collagen IV and collagen XVIII. VEGF inhibitors are disclosed in U.S. Pat. Nos. 6,534,524 and 6,235,764, both of which are incorporated in their entirety for all purposes. Anti-angiogenic agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known antiangiogenic factors); and, Sato (2003) Int. J Clin. Oncol. 8:200206 (e.g., Table 1 listing anti-angiogenic agents used in clinical trials).

The term “anti-angiogenic therapy” refers to a therapy useful for inhibiting angiogenesis which comprises the administration of an anti-angiogenic agent.

The terms “Iymphangiogenic” and “Iymphangiogenesis” relate to stimulation of the development of lymphatic vessels. Accordingly, a “Iymphangiogenic factor” is a growth factor and “Iymphangiogenic therapy” is therapy which stimulates the development of lymphatic vessels. Lymphangiogenic factors disclosed herein include, but are not limited to, VEGF-C and VEGF-D. It follows that “anti-Iymphangiogenic” relates to inhibition of lymphangiogenesis.

The term “toxin” refers to a substance capable of having a detrimental effect on the growth or proliferation of a cell.

The term “VEGF-C” refers to the full-length 419 amino acid polypeptide provided as SEQ ID NO: 1, together with the naturally occurring allelic, truncated and processed forms thereof, including active fragments of the full-length polypeptide. Active fragments include any portions of the full-length amino acid sequence SEQ ID NO: 1, which have VEGF-C biological activity, and which include, but are not limited to, mature VEGF-C. VEGF-C is synthesized as a precursor protein containing N-terminal and C-terminal propeptides in addition to the central VEGF homology domain (VHD) which contains the binding sites for VEGFR-2 and VEGFR-3. The N- and C-propeptides are proteolytically cleaved from the VHD during biosynthesis by proprotein convertases to generate a fully-processed mature form. In humans, the mature proteolytically processed form of VEGF-C can exist as a homodimer and binds to VEGFR-2 and VEGFR-3. The processed mature VEGF-C has binding affinity for the VEGFR-2 and VEGFR-3 receptors. Experimental evidence demonstrates that the full-length form, the partially-processed forms and the fully-processed mature forms of VEGF-C are able to bind VEGFR-3. High affinity binding to VEGFR-2, however, occurs only with the fully-processed mature forms of VEGF-C. The term “VEGF-C” is also used to refer to allelic, processed and truncated forms of VEGF-C derived from species other than human.

VEGF-C is a member of a structurally related VEGF family of angiogenic regulators. In addition to an angiogenic activity, VEGF-C appears to be involved in regulation of lymphangiogenesis via its binding to VEGFR-3. The angiogenesis induced by VEGF-C in tumors can promote solid tumor growth and metastatic spread, and the lymphangiogenesis induced by VEGF-C can promote metastatic spread of tumor cells to the lymphatic vessels and lymph nodes. Furthermore, clinicopathological data indicates a role for this growth factor in a range of prevalent human cancers. For example, levels of VEGF-C mRNA in lung cancer are associated with lymph node metastasis and in breast cancer correlate with lymphatic vessel invasion and shorter disease-free survival.

The terms “biological activity” and “biologically active” with regards to a VEGF-C polypeptide refer to physical/chemical properties and biological functions associated with full-length and/or mature VEGF-C. In some embodiments, VEGF-C “biological activity” means having the ability to bind to and stimulate the phosphorylation of VEGFR-2 and/or VEGFR-3. Generally, VEGF-C will bind to the extracellular domain(s) of VEGFR-2 and/or VEGFR-3 and thereby activate or inhibit the intracellular tyrosine kinase domain. Consequently, binding of VEGF-C to its receptor(s) may result in enhancement or inhibition of proliferation and/or differentiation and/or activation of cells possessing VEGFR-2 and/or VEGFR-3 on their surface in vivo or in vitro. Binding of VEGF-C to VEGFR-2 and/or VEGFR-3 can be determined using conventional techniques, including competitive binding methods, such as RIAs, ELISAs and other competitive binding assays. Ligand/receptor complexes can be identified using such separation methods as filtration, centrifugation, flow cytometry and the like. Results from binding studies can be analyzed using any conventional graphical representation of the binding data. Since VEGF-C induces phosphorylation of VEGFR-2 and VEGFR-3, conventional tyrosine phosphorylation assays can also be used as an indication of the formation of a VEGFR-2/VEGF-C and VEGFR-3/VEGF-C complex, respectively. In another embodiment, VEGF-C “biological activity” means having the ability to bind to VEGFR-2.

The term “VEGF-C antagonist” or refers to a molecule capable of reducing VEGF-C expression levels or neutralizing, blocking, inhibiting, abrogating, reducing or interfering with one or more of VEGF-C's biological activities, including but not limited to, VEGF-C binding to one or more of its receptors and VEGF-C mediated angiogenesis, lymphatic endothelial cell (LEC) migration, LEC proliferation or adult lymphangiogenesis. VEGF-C antagonists useful in the present invention include, without limitation, polypeptides that specifically bind to VEGF-C, VEGF-C antibodies and antigen-binding fragments thereof, polypeptides that bind VEGF-C and a VEGF-C receptor and block ligand-receptor interaction (e.g. immunoadhesins, peptibodies), VEGF receptor molecules and derivatives which bind to and sequester VEGF-C thereby preventing VEGF-C binding to and activating receptors expressed on cells in vivo (e.g. soluble receptor traps derived from VEGFR-2 and/or VEGFR-3), VEGF-C receptor antibodies, VEGF-C receptor antagonists such as small molecule inhibitors of the VEGFR-2 and VEGFR-3. The VEGF-C specific antagonists also include antagonist variants of VEGF-C polypeptides, antisense nucleobase oligomers directed to VEGF-C, small RNA molecules directed to VEGF-C, RNA aptamers, peptibodies and ribozymes against VEGF-C. The VEGF-C specific antagonists also include non-peptide small molecules that bind to VEGF-C and which are capable of blocking, inhibiting, abrogating, reducing or interfering with one or more VEGF-C biological activity. The term “VEGF-C activities” specifically includes VEGF-C mediated biological activities (as hereinabove defined) of VEGF-C. In certain embodiments, the VEGF-C antagonist reduces or inhibits, by at least 10%, 20%, 30%, 40%, 50%, 60%, 70%, 80%, 90% or more, the expression level or biological activity of VEGF-C. According to one preferred embodiment, the VEGF-C antagonist binds to VEGF-C and inhibits VEGF-C induced endothelial cell proliferation in vitro. According to one preferred embodiment the VEGF-C antagonist binds to a VEGF-C polypeptide or a VEGF-C receptor with greater affinity than it does to a non-VEGF-C polypeptide or non-VEGF-C receptor, respectively. According to another preferred embodiment the VEGF-C antagonist specifically binds to a VEGF-C polypeptide or a VEGF-C receptor. According to one preferred embodiment, the VEGF-C antagonist binds to VEGF-C or a VEGF-C receptor with a Kd of between 1 μM and 1 pM. According to another preferred embodiment, the VEGF-C antagonist binds to VEGF-C or a VEGF-C receptor with a Kd of between 500 nM and 1 pM. The term “VEGF-C antagonist” specifically includes molecules, including antibodies, antibody fragments, other binding polypeptides, peptides, and non-peptide small molecules, that bind to VEGF-C and are capable of neutralizing, blocking, inhibiting, abrogating, reducing or interfering with VEGF-C activities.

A VEGF-C antagonist may me a tyrosine kinase inhibitor (TKI) that inhibits VEGFR-3 activity. A TKI that inhibits VEGFR-3 activity means an inhibitor of receptor tyrosine kinase activity that selectively or non-selectively reduces the tyrosine kinase activity of a VEGFR-3 receptor. Such an inhibitor generally reduces VEGFR-3 tyrosine kinase activity without significantly effecting the expression of VEGFR-3 and without effecting other VEGFR-3 activities such as ligand-binding capacity. A VEGFR-3 kinase inhibitor can be a molecule that directly binds the VEGFR-3 catalytic domain, for example, an ATP analog. A VEGFR-3 kinase inhibitor can bind the VEGFR-3 catalytic domain through one or more hydrogen bonds similar to those anchoring the adenine moiety of ATP to VEGFR-3 (Engh et al., J. Biol. Chem. 271:26157-26164 (1996); Tong et al., Nature Struc. Biol. 4:311-316 (1997); and Wilson et al., Chem. Biol. 4:423-431 (1997)). A VEGFR-3 kinase inhibitor also can bind the hydrophobic pocket adjacent to the adenine binding site (Mohamedi et al., EMBO J. 17:5896-5904 (1998); Tong et al., supra, 1997; and Wilson et al., supra, 1997).

VEGFR-3 kinase inhibitors useful in the invention include specific VEGFR-3 kinase inhibitors such as indolinones that differentially block VEGF-C and VEGF-D induced VEGFR-3 kinase activity compared to that of VEGFR-2. Such specific VEGFR-3 kinase inhibitors, for example, MAE106 and MAZ51 can be prepared as described in Kirkin et al., Eur. J. Biochem. 268:5530-5540 (2001). Additional VEGFR-3 kinase inhibitors, including specific, selective and non-selective inhibitors, are known in the art or can be identified using one of a number of well known methods for assaying for receptor tyrosine kinase inhibition.

As an example, a VEGFR-3 kinase inhibitor can be identified using a well known ELISA assay to analyze production of phosphorylated tyrosine as described, for example in Hennequin et al., J. Med. Chem. 42:5369-5389 (1999) and Wedge et al., Cancer Res. 60:970-975 (2000). Such an assay can be used to screen for molecules that inhibit VEGFR-3 in preference to other vascular endothelial growth factor receptors such as VEGFR-1 and in preference to unrelated tyrosine kinases such as fibroblast growth factor receptor1 (FGFR1). Briefly, molecules to be screened can be incubated for 20 minutes at room temperature with a cytoplasmic receptor domain in a HEPES (pH 7.5) buffered solution containing 10 mM MnCl₂ and 2 μM ATP in 96-well plates coated with a poly(Glu, Ala, Tyr) 6:3:1 random copolymer substrate (SIGMA; St. Louis, Mo.). Phosphorylated tyrosine can be detected by sequential incubation with mouse IgG anti-phosphotyrosine antibody (Upstate Biotechnology; Lake Placid, N.Y.), a horseradish peroxidase-linked sheep anti-mouse immunoglobulin antibody (Amersham; Piscataway, N.J.), and 2,2′azino-bis (3-ethylbenzthiazoline-6-sulfonic acid) (Roche Molecular Biochemicals, Indianapolis, Ind.). In such an in vitro kinase assay, the source of VEGFR-3 can be, for example, a lysate prepared from an insect cell infected with recombinant baculovirus containing a cytoplasmic receptor domain, for example, encoding residues 798 to 1363 of human VEGFR-3.

The term VEGFR-3 kinase inhibitor, as used herein, encompasses specific, selective and non-selective inhibitors of VEGFR-3. A specific VEGFR-3 kinase inhibitor reduces the tyrosine kinase activity of VEGFR-3 in preference to the activity of most or all unrelated receptor tyrosine kinases such as FGFR1 and in preference to the activity of the vascular endothelial growth factor receptors, VEGFR-1 and VEGFR-2. A selective VEGFR-3 kinase inhibitor reduces the tyrosine kinase activity of VEGFR-3 in preference to most or all unrelated receptor tyrosine kinases such as FGFR1. Such a selective VEGFR-3 inhibitor can have an IC₅₀ for inhibition of an isolated VEGFR-3 cytoplasmic domain that is, for example, at least 10-fold less than the IC₅₀ for both VEGFR-1 and VEGFR-2. In particular embodiments, the invention provides a selective VEGFR-3 kinase inhibitor having an IC₅₀ for inhibition of an isolated VEGFR-3 cytoplasmic domain that is at least 20-fold, 30-fold, 40-fold, 50-fold, 100-fold, 200-fold, 300-fold, 400-fold or 500-fold less than the IC₅₀ for both VEGFR-1 and VEGFR-2. In contrast, a non-selective VEGFR-3 kinase inhibitor reduces the tyrosine kinase activity of VEGFR-1 or VEGFR-2 or both to a similar extent as VEGFR-3.

The term “VEGF-C antibody” refers to an antibody that binds to VEGF-C or a biologically active fragment thereof, e.g. the mature fully-processed form, with sufficient affinity and specificity. In a certain embodiment, a VEGF-C antibody is capable of binding VEGF-C with sufficient affinity such that the antibody is useful as a diagnostic and/or therapeutic agent targeting VEGF-C. The antibody may be subjected to biological activity assays in order to evaluate its effectiveness as a therapeutic. Such assays are known in the art and depend on the target antigen and intended use for the antibody. Examples include the HUVEC inhibition assay; tumor cell growth inhibition assays (e.g. as described in WO 89/06692); antibody-dependent cellular cytotoxicity (ADCC) and complement-mediated cytotoxicity (CDC) assays (e.g. as described is U.S. Pat. No. 5,500,362); and agonistic activity or hematopoiesis assays (e.g. as described in WO95/27062). The binding of a VEGF-C antibody will partially or fully block, neutralize, reduce or antagonize VEGF-C activity. A VEGF-C antibody will usually not bind to other VEGF homologues such as VEGF-A, VEGF-B, VEGF-D or VEGF-E, nor other growth factors such as PIGF, PDGF or bFGF. In one embodiment, the extent of binding of a VEGF-C antibody to an unrelated, non-VEGF-C protein is less than about 10% of the binding of the antibody to VEGF-C as measured, e.g., by a radioimmunoassay (RIA). In a preferred embodiment the VEGF-C antibody specifically binds to a VEGF-C polypeptide. In certain embodiments, an antibody that binds to VEGF-C has a dissociation constant (Kd) of ≦1 μM, ≦100 nM, ≦10 nM, ≦1 nM or ≦0.1 nM. In certain embodiments, the antibody selected will have a binding affinity for hVEGF-C with a Kd value of between 100 nM-1 pM. Antibody affinities may be determined, for example, by a surface Plasmon resonance based assay (such as the BIAcore assay as described WO 2005/012359); enzyme-linked immunoabsorbent assay (ELISA); and competition assays (e.g. RIA's). In certain embodiments, a VEGF-C antibody binds to an epitope of VEGF-C that is conserved among VEGF-C from different species.

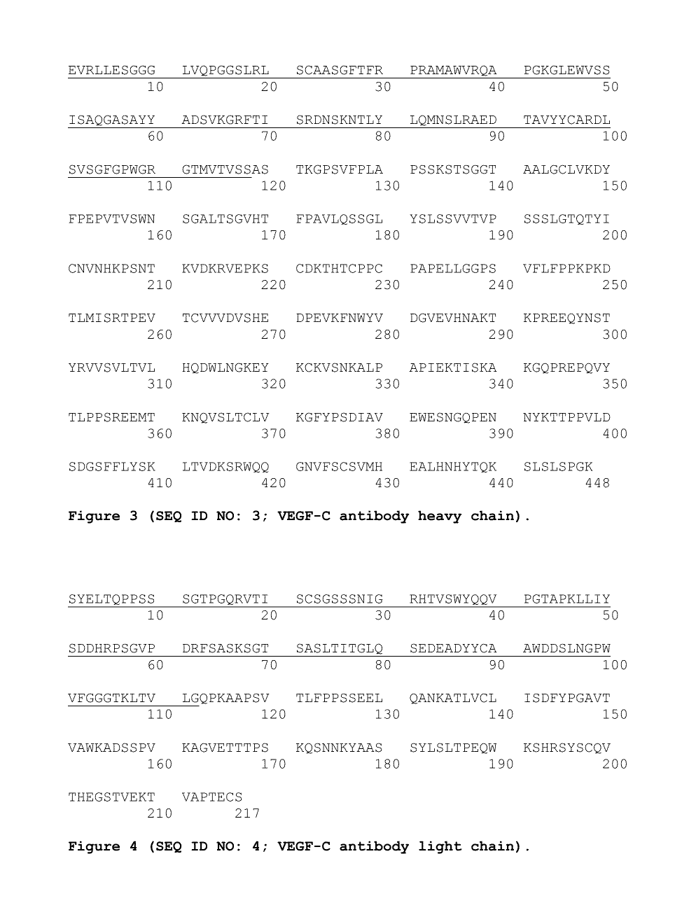

One example of a VEGF-C antibody is a monoclonal antibody that competitively inhibits the binding to VEGF-C of monoclonal VEGF-C antibody 69D09 produced by hybridoma ATCC PTA-4095 or having the heavy and light chain amino acid sequences shown in FIGS. 3 and 4, respectively. Another example of a VEGF-C antibody is a monoclonal antibody that binds to the same epitope as the monoclonal VEGF-C antibody 69D09 produced by hybridoma ATCC PTA-4095 or a monoclonal antibody having the heavy and light chain amino acid sequences shown in FIGS. 3 and 4, respectively. In one embodiment, the VEGF-C antibody is a fully-human VEGF-C monoclonal antibody, including but not limited to 69D09 antibody or fragment thereof. Other examples of suitable antibodies include antibodies 103, MM0006-2E65 and 193208. Further examples of such antibodies are found in U.S. Pat. Nos. 7,208,582, 7,850,963 and 7,109,308. The VEGF-C antibody may be a humanized antibody. Preferably, the VEGF-C antibody is a human antibody produced by deposited hybridoma ATC PTA-4095 or having the heavy and light chain amino acid sequences shown in FIGS. 3 and 4, respectively.

The term “VEGF-A” refers to the 232-amino acid polypeptide provided as SEQ ID NO: 2, together with naturally occurring allelic, truncated and and processed forms thereof. More particularly, the term “VEGF-A” as used herein refers to the 165-amino acid isoform of human VEGF-A and the related 121-, 189-, and 206-amino acid isoforms, as described by Leung et al. (1989) Science 246: 1306, and Houck et al. (1991) Mol. Endocrin, 5:1806, together with the naturally occurring allelic and processed forms thereof. The term “VEGF-A” is also used to refer to truncated forms of the polypeptide comprising amino acids 8 to 109 or 1 to 109 of the 165-amino acid human VEGF-A isoform. The term “VEGF-A” is also used to refer to allelic, processed and truncated forms of VEGF-A, including the various isoforms, derived from species other than human, such as mouse, rat and primate. VEGF-A has binding affinity for the VEGFR-1 (Flt-1) and VEGFR-2 (KDR).

The terms “biological activity” and “biologically active” with regards to a VEGF-A polypeptide refer to binding to any VEGF-A receptor or to any VEGF-A signaling activity such as regulation of both normal and abnormal angiogenesis and vasculogenesis (Ferrara and Davis-Smyth (1997) Endocrine Rev. 18:4-25; Ferrara (1999)J. Mol. Med. 77:527-543); promoting embryonic vasculogenesis and angiogenesis (Carmeliet et al. (1996) Nature 380:435-439; Ferrara et al. (1996) Nature 380: 439-442); and modulating the cyclical blood vessel proliferation in the female reproductive tract and for bone growth and cartilage formation (Ferrara et al. (1998) Nature Med. 4:336340; Gerber et al. (1999) Nature Med. 5:623-628). In addition to being an angiogenic factor in angiogenesis and vasculogenesis, VEGF-A, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life. Sci. 63:601-615 (2006)). Moreover, recent studies have reported mitogenic effects of VEGF-A on a few non-endothelial cell types, such as retinal pigment epithelial cells, pancreatic duct cells, and Schwarm cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

The term “VEGF-A antagonist” or refers to a molecule capable of reducing VEGF-A expression levels or neutralizing, blocking, inhibiting, abrogating, reducing or interfering with one or more of VEGF-A's biological activities, including but not limited to, VEGF-A binding to one or more of its receptors and VEGF-A mediated angiogenesis. Preferably, the VEGF-A specific antagonist binds VEGF-A or a VEGF-A receptor (e.g. VEGFR-1 or VEGFR-2). VEGF-A specific antagonists useful in the present invention include, without limitation, polypeptides that specifically bind VEGF-A, VEGF-A antibodies and antigen-binding fragments thereof, VEGF receptor molecules and derivatives which bind to and sequester VEGF-A thereby preventing VEGF-A binding to and activating receptors expressed on cells in vivo (e.g. soluble receptor traps derived from VEGFR-1 and/or VEGFR-2 such as, for example, the VEGF-Trap from Regeneron), polypeptides that bind VEGF-A and a VEGF-A receptor and block ligand-receptor interaction (e.g. Immunoadhesins, peptibodies), VEGF-A receptor antibodies, VEGF-A receptor antagonists such as small molecule inhibitors of the VEGFR-1 and/or VEGFR-2, fusion proteins such as VEGF₁₂₁-gelonin (Peregrine), antagonist variants of VEGF-A polypeptides, aptamers that bind VEGF-A, antisense nucleobase oligomers directed to VEGF-A or a VEGF-A receptor, small RNA molecules directed to VEGF-A or a VEGF-A receptor (e.g. RNAi), and ribozymes against VEGF-A. The VEGF-A specific antagonists also include non-peptide small molecules that bind to VEGF-C and which are capable of blocking, inhibiting, abrogating, reducing or interfering with one or more of VEGF-A's biological activities. Thus, the term “VEGF-A activities” specifically includes VEGF-A mediated biological activities of VEGF-A. In certain embodiments, the VEGF-A antagonist reduces or inhibits, by at least 10%, 20%, 30%, 40%, 50%, 60%, 70%, 80%, 90% or more, the expression level or one or more biological activities of VEGF-A. According to one preferred embodiment, the VEGF-A antagonist binds to VEGF and inhibits VEGF-A induced endothelial cell proliferation in vitro. According to another preferred embodiment the VEGF-A antagonist specifically binds to a VEGF-A polypeptide or a VEGF-A receptor. According to one preferred embodiment the VEGF-A antagonist binds to a VEGF-A polypeptide or a VEGF-A receptor with greater affinity than it does to a non-VEGF-A polypeptide or non-VEGF-A receptor, respectively. According to one preferred embodiment, the VEGF-A antagonist binds to VEGF-A or a VEGF-A receptor with a Kd of between 1 μM and 1 pM. According to another preferred embodiment, the VEGF-A antagonist binds to VEGF-A or a VEGF-A receptor with a Kd of between 500 nM and 1 pM.

According a preferred embodiment, the VEGF-A antagonist is selected from a polypeptide such as an antibody, a peptibody, an immunoadhesin, a small molecule or an aptamer. In a preferred embodiment, the antibody is a VEGF-A antibody such as the AVASTIN® antibody or a VEGF-A receptor antibody such as a VEGFR-1 or a VEGFR-2 antibody antibody. Other examples of VEGF antagonists include: VEGF-Trap, Mucagen, PTK787, SUI 1248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

The term “VEGF-A antibody” refers to an antibody that binds to VEGF-A with sufficient affinity and specificity. In a certain embodiment, a VEGF-A antibody is capable of binding VEGF-A with sufficient affinity such that the antibody is useful as a diagnostic and/or therapeutic agent targeting VEGF-A. Preferably, the VEGF-A antibody can be used as a therapeutic agent in targeting and interfering with diseases or conditions in which VEGF-A activity is involved. The antibody may be subjected to biological activity assays in order to evaluate its effectiveness as a therapeutic. Such assays are known in the art and depend on the target antigen and intended use for the antibody. Examples include the HUVEC inhibition assay; tumor cell growth inhibition assays (e.g. as described in WO 89/06692); antibody-dependent cellular cytotoxicity (ADCC) and complement-mediated cytotoxicity (CDC) assays (e.g. as described is U.S. Pat. No. 5,500,362); and agonistic activity or hematopoiesis assays (e.g. as described in WO95/27062). The binding of a VEGF-A antibody will partially or fully block, neutralize, reduce or antagonize VEGF-A activity. The VEGF-A antibody will usually not bind to other VEGF-A homologues such as VEGF-B, VEGF-C, VEGF-D or VEGF-E, nor other growth factors such as PIGF, PDGF or bFGF. In one embodiment, the extent of binding of a VEGF-A antibody to an unrelated, non-VEGF-A protein is less than about 10% of the binding of the antibody to VEGF-A as measured, e.g., by a radioimmunoassay (RIA). In a preferred embodiment, the VEGF-A antibody specifically binds to a VEGF-A polypeptide. In certain embodiments, an antibody that binds to VEGF-A has a dissociation constant (Kd) of ≦5 μM, ≦100 nM, ≦10 nM, ≦1 nM or ≦0.1 nM. In certain embodiments, the antibody selected will have a binding affinity for hVEGF-A with a Kd value of between 100 nM-1 pM. Antibody affinities may be determined by a surface Plasmon resonance based assay (such as the BIAcore assay as described in WO 2005/012359), enzyme-linked immunoabsorbent assay (ELISA); and competition assays (e.g. RIA's). In certain embodiments, a VEGF-A antibody binds to an epitope of VEGF-A that is conserved among VEGF-A from different species. In a preferred embodiment, the VEGF-A antibody is a monoclonal antibody that binds to the same epitope as the monoclonal VEGF-A antibody A4.6.I produced by hybridoma ATCC HB 10709. More preferably, the VEGF-A antibody is a recombinant humanized VEGF-A monoclonal antibody generated according to Presta et al. (1997) Cancer Res. 57:4593-4599.

A particularly preferred VEGF-A antibody generated according to Presta et al, is the antibody known as “Bevacizumab (BV),” or “AVASTIN®” (also known as “rhuMAb VEGF”). Bevacizumab comprises mutated human IgGI framework regions and antigen-binding complementarity-determining regions (CDRs) from the murine hVEGF-A monoclonal antibody AA.6.1 that blocks binding of humanVEGF-A to its receptors. Approximately 93% of the amino acid sequence of Bevacizumab, including most of the framework regions, is derived from human IgGI, and about 7% of the sequence is derived from the murine antibody A4.6.1. Bevacizumab has a molecular mass of about 149,000 daltons and is glycosylated. Bevacizumab and other humanized VEGF-A antibodies are further described in U.S. Pat. No. 6,884,879, the entire disclosure of which is expressly incorporated herein by reference.

In another empbidment, the VEGF-A antibody is Ranibizumab (LUCENTIS® antibody or rhuFab V2). Ranibizumab is a humanized, affinity-matured humanVEGF-A Fab fragment produced by standard recombinant technology methods in Escherichia coli expression vector and bacterial fermentation. Ranibizumab is not glycosylated and has a molecular mass of −48,000 daltons. See WO 98/45331 and US 20030190317.

Other VEGF-A antibodies that may be used in the present invention include antibodies derived from a sequence of the B20 or G6 antibody as described in US 20060280747, US 20070141065 and US 20070020267, the entire contents of which are expressly incorporated herein by reference. In one embodiment, the antibody is B20-4.1 as described in US 20060280747, US 20070141065 and US 20070020267. In another embodiment, the antibody is B20-4.1.1 as described in U.S. 60/991,302, the entire contents of which are expressly incorporated herein by reference. Potentially useful G6-derived antibodies include, but are not limited to, G6-8, G6-23 and G6-31.

Other additional VEGF-A antibodies are described in U.S. Pat. No. 7,060,269, U.S. Pat. No. 6,582,959, U.S. Pat. No. 6,703,020, U.S. Pat. No. 6,054,297, WO 96/30046, WO 94/10202, EP 666868, US 2006009360, US20050186208, US 20030206899, US 20030190317, US 20030203409, US 20050112126 and Popkov et al. Journal of Immunological Methods 288: 149-164 (2004).

VEGF receptors include VEGFR-1 (also known as Flt-1), VEGFR-2 (also known as KDR and FLK-1 for the murine homolog) and VEGFR-3 (also known as Flt-4). The specificity of each receptor for each VEGF family member varies. VEGF-A binds both VEGFR-1 and VEGFR-2. VEGF-C binds VEGFR-2 and VEGFR-3. The full length receptors comprise an extracellular domain having seven Ig-like domains, a transmembrane domain and an intracellular domain with tyrosine kinase activity. The extracellular domains are involved in the binding of the VEGF ligands and the intracellular domains are involved in signal transduction.

VEGF receptor molecules, or fragments thereof, that specifically bind to a VEGF ligand can be used as VEGF antagonists that bind to and sequester the VEGF protein, thereby preventing it from signaling. In a preferred embodiment, the VEGF receptor molecule is soluble. A soluble VEGF receptor molecule exerts an inhibitory effect on the biological activity of the VEGF protein by binding to the protein, thereby preventing it from binding to its natural receptors present on the surface of target cells. It is undesirable for a VEGF receptor molecule not to become associated with the cell membrane. In a preferred embodiment, the soluble VEGF receptor molecule lacks any amino acid sequences corresponding to the transmembrane and intracellular domains from the VEGF receptor(s) form which it is derived.

The VEGF receptor molecule may be a chimeric molecule comprising amino acid sequences derived from at least two different receptor proteins, at least one of which is a VEGF receptor protein (i.e. VEGFR-1, VEGFR-2 or VEGFR-3) that is capable of binding to and inhibiting the biological activity of a VEGF. In certain embodiments, a chimeric VEGF receptor molecule contains amino acid sequences derived from only two different receptor proteins selected from VEGFR-1, VEGFR-2 and VEGFR-3. In a preferred embodiment, a VEGF-receptor molecule only comprises one, two, three, four, five, six, or all seven Ig-like domains from the extracellular ligand-binding region of VEGFR-1, VEGFR-2 or VEGFR-3. Preferably, the VEGF receptor molecule comprises one or more Ig-like domains and lacks any transmembrane and intracellular domains from VEGFR-1, VEGFR-2 and VEGFR-3. In a particularly preferred embodiment, that part of the VEGF receptor molecule derived from VEGFR-1, VEGFR-2 and/or VEGFR-3, is selected from Ig-like domains one, two and three, or derivatives of these Ig-like domains.

The VEGF receptor molecule may be a fusion protein in which the amino acid sequences derived from the receptor protein(s) (e.g. the Ig-like domains derived from VEGFR-2, VEGFR-2 and/or VEGFR-3) are linked to amino acids from an unrelated protein, for example, immunoglobulin sequences. In a preferred embodiment, the amino acid sequences derived from the receptor protein(s) are fused to an Fc portion of an immunoglobulin. Other amino acid sequences to which Ig-like domains may be combined (fused) will be apparent to the skilled person in the art.

Examples of VEGF receptor molecules useful in the present invention include soluble VEGFR-1/Fc (comprising Ig-like domains from the extracellular domain of VEGFR-1 fused to an Ig Fc), VEGFR-2/Fc (comprising Ig-like domains from the extracellular domain of VEGFR-2 fused to an Ig Fc) and VEGFR-1/VEGFR-2/Fc (comprising Ig-like domains from the extracellular domains of VEGFR-1 and VEGFR-2 fused to an Ig Fc) (also known as the VEGF-Trap from Regeneron)—see WO 97/44453). These VEGF receptor molecules sequester VEGF-A.

Examples of soluble VEGF receptor molecules which sequester VEGF-C, thereby inhibiting VEGF-C activity or signaling via VEGFR-2 and VEGFR-3, are disclosed in WO2000/023565, WO2000/021560, WO2002/060950 and WO 2005/087808. Such inhibitors of VEGF-C activity include soluble VEGFR-2-, VEGFR-3- and NRP-2 derived traps. In a preferred embodiment, the VEGF-C antagonist is a soluble VEGF-C receptor molecule. A preferred VEGF-C receptor molecule is a polypeptide comprising a portion of the extracellular domain of VEGFR-3, the portion comprising at least Ig-like domains 1-3 of the extracellular domain and lacking Ig-like domain s 4-7 and the polypeptide lacking any transmembrane domain. These constructs are described in more detail in WO 2002/060950.

It will be appreciated that certain antagonists, in particular those targeting or derived from a VEGF receptor, may antagonize more than one VEGF ligand. For example, a VEGFR-3 antibody or a VEGF receptor molecule derived from VEGFR-3 could be used to inhibit (antagonize) the activity of VEGF-C and VEGF-D, as both these VEGF ligands bind to VEGFR-3. In the case of an antagonist molecule which can bind to two VEGF/PGDF family ligands, e.g. a soluble VEGFR-3 receptor trap which is capable of binding and sequestering both VEGF-C and VEGF-D polypeptides, then reference herein to the antagonist molecule specifically binding refers to the ability of the antagonist to bind to both ligands. By way of example and with reference to an antagonist molecule which binds to and antagonises X and Y (e.g. neutralizes, blocks, inhibits, abrogates, reduces or interferes with one or more of the biological activities of X and Y), in one embodiment, the antagonist reduces or inhibits, by at least 10%, 20%, 30%, 40%, 50%, 60%, 70%, 80%, 90% or more, the expression level or biological activity of X and Y. According to another embodiment, the antagonist (i) binds to X and inhibits a biological activity of X (e.g. binds to VEGF-C and inhibits VEGF-C induced endothelial cell proliferation in vitro) and (ii) binds to Y and inhibits a biological activity of Y (e.g. binds to VEGF-D and inhibits VEGF-D induced lymphatic endothelial cell proliferation in vitro). According to another preferred embodiment, the antagonist binds to X and Y separately with greater affinity than it does to both a non-X and non-Y polypeptide. According to another preferred embodiment, the antagonist specifically binds to both X and Y. According to one preferred embodiment, the antagonist binds to both X and Y separately with a Kd of between 1 μM and 1 pM. According to another preferred embodiment, the antagonist binds to X and Y separately with a Kd of between 500 nM and 1 pM.

For the avoidance of doubt, reference herein to use of, administration of or a product comprising a VEGF-C antagonist and an anti-neoplastic composition means the use of, administration of or a product comprising two different compounds.

The term “antagonist” refers to a compound or agent which inhibits or reduces the biological activity of the molecule to which it binds. Antagonists include antibodies, synthetic or native-sequence peptides, immunoadhesins, and small-molecule antagonists that bind to VEGF, optionally conjugated with or fused to another molecule. A “blocking” antibody or an “antagonist” antibody is one which inhibits or reduces biological activity of the antigen to which it binds.

The terms “treat”, “treating” or “treatment” refer to both therapeutic treatment and prophylactic or preventative measures, wherein the aim is to prevent or ameliorate cancer or slow down (lessen) cancer progression. Those in need of treatment include those already with the disorder as well as those in which the disorder is to be prevented.

The terms “preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be in whom the condition is to be prevented.

The term “standard of care” or “best practice” refers to treatment that experts agree is appropriate, accepted, and widely used in respect of a certain set of symptoms or a specific disorder, such as a cancer. The person skilled in the art will be aware of the standard of care for any given cancer. The standard of care may be different for different types of cancer or for different stages of the same type of cancer.

The term “resistant cancer” or “resistant tumor” refers to cancer, cancerous cells, or a tumor that does not respond completely, or loses or shows a reduced response over the course of cancer therapy to a cancer therapy comprising at least a VEGF-A antagonist. In certain embodiments, resistant tumor is a tumor that is resistant to a VEGF-A antibody therapy. In one embodiment, the VEGF-A antibody is bevacizumab. In certain embodiments, a resistant tumor is a tumor that is unlikely to respond to a cancer therapy comprising at least a VEGF-A antagonist.

Reference to administration “in combination” refers to simultaneous (concurrent) and consecutive administration in any order. The combined administration includes co-administration, using separate formulations or a single pharmaceutical formulation, and consecutive administration in any order. Preferably there is a time period while both (or all) active agents simultaneously exert their biological activities.

The term “subject” refers to a mammal. The mammal may be a primate, particularly a human, or may be a domestic, zoo, or companion animal. While it is particularly contemplated that the method and article of manufacture disclosed herein are suitable for medical treatment of humans, they are also applicable to veterinary treatment, including treatment of domestic animals such as horses, cattle and sheep, companion animals such as dogs and cats, or zoo animals such as felids, canids, bovids and ungulates.

The term “therapeutically effective amount” refers to an amount of a drug effective to treat a disease, disorder or phenotype in a mammal. In the case of cancer, the therapeutically effective amount of the drug may: reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder or disease. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life. In combination a “therapeutically effective amount” is such that administration of the VEGF-C antagonist and the anti-neoplastic composition (comprising one or more other therapeutic agents) results in reduction or inhibition of the targeted disorder.

The term a “therapeutically synergistic amount” refers to that amount of VEGF-C antagonist and the anti-neoplastic composition necessary to synergistically reduce or eliminate conditions or symptoms associated with the targeted disorder.

The term “antibody” is used in its broadest sense and specifically includes monoclonal antibodies (including full length or intact monoclonal antibodies), polyclonal antibodies, multivalent antibodies, multispecific antibodies (e.g., bispecific antibodies), and antibody fragments (see below) so long as they exhibit the desired biological activity.

The term “specific binding” or “specifically binds” or “specific for” refers to binding where a molecule binds to a particular polypeptide or epitope on a particular polypeptide without substantially binding to any other polypeptide or polypeptide epitope. Such binding is measurably different from a non-specific interaction. For example, specific binding can be determined using competition assays. As used herein, specific binding is used in relation to the interaction between an antibody and a polypeptide or polypeptide epitope and to the interaction between a receptor and a polypeptide or other receptor-binding molecule. Specific binding also applies to molecular interactions that partially or fully block, neutralize, reduce or antagonize VEGF-C or VEGF-A biological activity.

In particular, “specific binding” or “specifically binds” or “specific for” refers to a molecule having a Kd value at least 2-fold less for the particular polypeptide or polypeptide epitope than it does for a non-specific target. Specific binding also refers to a molecule having a Kd value at least 4-fold, 6-fold, 8-fold, 10-fold or greater than 10-fold less for the particular polypeptide or polypeptide epitope than it does for a non-specific target. Alternatively, specific binding can be expressed as a molecule having a Kd value for the target of at least about 10⁻⁴ M, about 10⁻⁵ M, about 10⁻⁶ M, about 10⁻⁷ M, about 10⁻⁸ M, about 10⁻⁹ M, about 10⁻¹⁰ M, about 10⁻¹¹ M, about 10⁻¹²M, or less.

The term “binding affinity” generally refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

In one embodiment, the “Kd” or “Kd value” or “Kd constant” is measured by a radiolabeled antigen binding assay (RIA) performed with the Fab version of an antibody of interest and its antigen as described by the following assay. Solution-binding affinity of Fabs for antigen is measured by equilibrating Fab with a minimal concentration of ¹²⁵I-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest. The Fab of interest is then incubated overnight (incubation may continue for a longer period (e.g. about 65 hours) to ensure that equilibrium is reached). Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd or Kd value is measured by using surface-plasmon resonance assays using a BIACORE®-2000 or a BIACORE®-3000 instrument (BIAcore, Inc., Piscataway, N.J.) at 25° C. with immobilized antigen CM5 chips at −10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μl/minute to achieve approximately ten response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (Kₒₙ) and dissociation rates (K_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio K_{off}/kₒₙ. See, e.g., Chen et al., J. Mol. Biol 293:865-881 (1999). If the on-rate exceeds 10⁶ M⁻¹ s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette. An “on-rate,” “rate of association,” “association rate,” or “kₒₙ” can also be determined as described above using a BIACORE®-2000 or a BIACORE®-3000 system (BIAcore, Inc., Piscataway, N.J.).

Unless indicated otherwise, the expression “multivalent antibody” refers to an antibody comprising three or more antigen binding sites. The multivalent antibody may be engineered to have the three or more antigen binding sites and is generally not a native sequence IgM or IgA antibody.

The term “antibody fragment” refers to a molecule comprising only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment which consists of a VH domain; (vii) isolated complementarity determining regions (CDRs); (viii) F(ab′)₂ fragments, a bivalent fragment including two Fab′ fragments linked by a disulphide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd segments (VH-CH1-VH-CH1) which, together with complementary light chain polypeptides, form a pair of antigen binding regions.

The term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the present invention may be made by the hybridoma method, or may be made by recombinant DNA methods. The “monoclonal antibodies” may also be isolated from phage antibody libraries.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

Reference to “humanized” forms of non-human (e.g., murine) antibodies refer to chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin.

The term “human antibody” refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. Alternatively, the human antibody may be prepared via immortalization of human B lymphocytes producing an antibody directed against a target antigen (such B lymphocytes may be recovered from an individual or may have been immunized in vitro).

The term “affinity matured” antibody refers to an antibody with one or more alterations in one or more CDRs thereof which result an improvement in the affinity of the antibody for antigen, compared to a parent antibody which does not possess those alteration(s). In one embodiment, affinity matured antibodies will have nanomolar or even picomolar affinities for the target antigen. Affinity matured antibodies are produced by procedures known in the art, e.g. affinity maturation by VH and VL domain shuffling, or random mutagenesis of CDR and/or framework residues.

The term “isolated” with respect to a polypeptide, CDR, antibody or other entity, refers to a polypeptide, CDR, antibody or other entity that has been identified and separated and/or recovered from a component of its natural environment. Contaminant components of its natural environment are materials that would interfere with diagnostic or therapeutic uses for the polypeptide or antibody, and may include enzymes, hormones, and other proteinaceous or non-proteinaceous solutes. In some embodiments, the polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. An isolated polypeptide includes the polypeptide in situ within recombinant cells since at least one component of the polypeptide's natural environment will not be present. Ordinarily, however, an isolated polypeptide will be prepared by at least one purification step.

The term “biological characteristic” with regards to an antibody refers to the designated antibody possessing one or more of the biological characteristics of that antibody which distinguish it from other antibodies that bind to the same antigen. In order to screen for antibodies which bind to an epitope on an antigen bound by an antibody of interest, a routine cross-blocking assay can be performed.

The term “label” refers to a detectable compound or composition which is conjugated directly or indirectly to a polypeptide or antibody, for example. The label may be itself be detectable (e.g., radioisotope labels or fluorescent labels) or, in the case of an enzymatic label, may catalyze chemical alteration of a substrate compound or composition which is detectable.

As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

The term “duration of survival” refers to the time from first administration of the drug to death. “Duration of survival” can also be measured by stratified hazard ratio (HR) of a treatment group versus a control group, which represents the risk of death for a subject during the treatment.

The term “time to disease progression” refers to the time from administration of the therapy until disease progression.

The term “response rate” refers to the percentage of treated subjects who responded to the treatment.

The term “duration of response” refers to the time from the initial response to disease progression.

Therapeutic Uses of VEGF-C Antagonists

The present inventors have found that using a VEGF-C antagonist, and in particular a VEGF-C antibody, in combination with an anti-neoplastic composition provides significant benefits in the treatment of cancer.

Thus, in a first aspect, the present invention provides a method of treating cancer in a subject, comprising administering to the subject in combination therapeutically effective amounts of a VEGF-C antagonist and an anti-neoplastic composition.

In one form of the first aspect, the present invention provides a VEGF-C antagonist and an anti-neoplastic composition in combination for treating cancer in a subject.

In a second aspect, the present invention provides an article of manufacture (e.g. a kit) comprising a VEGF-C antagonist. The article of manufacture may comprise a container containing a VEGF-C antagonist, and a package insert instructing the user of the VEGF-C antagonist to administer to a subject with cancer the VEGF-C antagonist in combination with an anti-neoplastic composition.

In a preferred embodiment of the first or second aspect of the invention, the subject is human.

Cancers are typically defined by stages of progression. Overall Stage Grouping (also referred to as Roman Numeral Staging), uses numerals I, II, III and IV to describe the progression of the cancer. Broadly speaking, the stages are defined as follows. Stage I usually means the cancer is relatively small and contained within the organ in which it originated, i.e. the cancer is localized. Stage II usually means the cancer has not started to spread into surrounding tissue, but the tumor is larger than in stage I, i.e. the cancer is locally advanced. Sometimes stage II means that cancer cells have spread into lymph nodes close to the tumor. Stage III usually means the cancer is larger. It may have started to spread into surrounding tissues and there are cancer cells in the lymph nodes in the area. The cancer is still locally advanced but typically is now larger than in stage II. Stage IV usually means the cancer has spread or metastasized to other organs or throughout the body—this is also called metastatic cancer. This stage usually represents inoperable cancer. Although these stages are defined precisely, the definition is different for each kind of cancer.

Unfortunately, it is common for cancer to return months or years after the primary tumor has been removed because cancer cells had already broken away and lodged in distant locations by the time the primary tumor was discovered, but had not formed tumors which were large enough to detect at that time. Sometimes a tiny bit of the primary tumor was left behind in the initial surgery and this later grows into a macroscopic tumor. Cancer that recurs after all visible tumor has been eradicated is called recurrent disease. Disease that recurs in the area of the primary tumor is locally recurrent, and disease that recurs as metastases is referred to as a distant recurrence. Distant recurrence is usually treated similarly to stage IV disease (sometimes the terms are used interchangeably). The significance of local recurrence may be quite different than distant recurrence, depending on the type of cancer.

The method of the first aspect and the article of manufacture of the second aspect may be used in the treatment of any of the cancers described above and at any stage of the cancer. The method of the first aspect and the article of manufacture of the second aspect may be used in the treatment of stage I, II, III or IV cancers or in a treatment regime adopted after surgery (e.g. resection) or in the treatment of recurring cancer. In one embodiment, it is envisaged that the VEGF-C antagonist will be useful in treatment of an early stage cancer. Without being limited by theory, it is envisaged that using a VEGF-C antagonist in combination with an anti-neoplastic agent could benefit the patient insofar as the VEGF-C antagonist would inhibit the VEGF-C driven growth of the tumor thus enabling the anti-neopleastic to be more effective in attacking (e.g. shrinking or eradicating) the tumor.

It is envisaged that the method, article and use of the invention will be particularly useful in treating subjects in which the cancer has begun to spread from the organ of origin to local lymph nodes and beyond. For example, it is envisaged that the method, article and use of the invention will be particularly useful for treating subjects in which the cancer has metastasized to other tissues or parts (e.g. organs) of the body, optionally in addition to spreading to local and/or distant lymph nodes. It is envisaged that the method, article and use of the invention will be particularly useful for treating subjects with stage III or IV cancer, and/or subjects in which the tumor or tumors are no longer respectable.

The method of the first aspect or the article of manufacture of the second aspect is particularly suitable for the treatment of vascularized, solid tumors. In an embodiment of the first or second aspect, the cancer is selected from the group consisting of lung and bronchial cancers, colorectal cancers, prostate cancers, pancreatic cancers, liver cancers, esophageal cancers, urinary and bladder cancers, non-Hodgkin lymphomas, kidney and renal cancers, breast cancers, ovarian cancers and brain cancers (e.g. glioblastomas). In a preferred embodiment of the first or second aspect, the cancer is colorectal cancer (e.g. metastatic colorectal cancer), lung cancer (e.g. non-small cell lung cancer, NSCLC), prostate cancer, glioblastoma, kidney cancer (e.g. metastatic renal cell carcinoma, RCC), pancreatric cancer or ovarian cancer.

The method of the first aspect and the article of manufacture of the second aspect encompass an anti-angiogenic therapy, i.e. a novel cancer treatment strategy aimed at inhibiting the development of tumor blood vessels required for providing nutrients to support tumor growth. Because angiogenesis is involved in both primary tumor growth and metastasis, the anti-angiogenic treatment provided by the disclosure is capable of inhibiting the neoplastic growth of a tumor at the primary site as well as preventing metastasis of tumors at the secondary sites, therefore allowing attack of the tumors by other therapeutics.

The method of the first aspect and the article of manufacture of the second aspect may encompass an anti-lymphangiogenic therapy, i.e. a novel cancer treatment strategy aimed at inhibiting the development of lymphatic vessels which have been implicated in the metastatic spread of tumors.

The method of the first aspect and the article of manufacture of the second aspect encompass a combined anti-angiogenic and anti-lymphangiogenic therapy, providing the combined benefits of both individual therapies.

The VEGF-C antagonist may be any suitable molecule described herein. In a preferred embodiment, the VEGF-C antagonist is selected from a VEGF-C antibody, a VEGFR-3 antibody and a VEGFR-3 receptor molecule (i.e. a VEGR-3 receptor trap) as described herein.

In a highly preferred embodiment, the VEGF-C antagonist is a VEGF-C antibody as described herein. A preferred VEGF-C antibody is a monoclonal antibody that competitively inhibits the binding to VEGF-C of monoclonal VEGF-C antibody 69D09 produced by hybridoma ATCC PTA-4095 or having the heavy and light chain amino acid sequences shown in FIGS. 3 and 4, respectively. Another preferred VEGF-C antibody is a monoclonal antibody that binds to the same epitope as the monoclonal VEGF-C antibody 69D09 produced by hybridoma ATCC PTA-4095 or a monoclonal antibody having the heavy and light chain amino acid sequences shown in FIGS. 3 and 4, respectively. In one embodiment, the VEGF-C antibody is a fully-human VEGF-C monoclonal antibody, including but not limited to 69D09 antibody or fragment thereof. The VEGF-C antibody may be a humanized antibody. Preferably, the VEGF-C antibody is a human antibody produced by deposited hybridoma ATCC PTA-4095 or having the heavy and light chain amino acid sequences shown in FIGS. 3 and 4.

One or more therapeutic agents can be used in the anti-neoplastic composition to be administered in combination with administration of the VEGF-C antagonist. In one embodiment of the first or second aspect, the anti-neoplastic composition comprises a standard of care for the cancer to be treated.

It is contemplated that when used to treat various diseases such as tumors, the VEGF-C antagonist can be combined with other therapeutic agents suitable for the same or similar diseases. When used for treating cancer, the VEGF-C antagonists may be used in combination with conventional cancer therapies, such as surgery, radiotherapy, chemotherapy or combinations thereof. When used for treating cancer, the anti-neoplastic composition suitably comprises one or more therapeutic agents capable of inhibiting or preventing tumor growth or function, and/or causing destruction of tumor cells.

Other therapeutic agents useful for combination cancer therapy with the VEGF-C antagonists include other anti-angiogenic agents. Many anti-angiogenic agents have been identified and are known in the art. In another embodiment of the first or second aspect, the anti-neoplastic composition comprises an anti-angiogenic agent. Preferably, the anti-angiogenic agent is an anti-angiogenic antibody. In a preferred embodiment of the first or second aspect, the anti-neoplastic composition comprises a VEGF-A antagonist. Preferred VEGF-A antagonists are selected from VEGF-A antibodies and soluble VEGF receptor molecules specific for VEGF-A (e.g. such as the VEGF Trap from Regeneron). A preferred VEGF-A antibody is Bevacizumab (Avastin).

In one embodiment, the VEGF-C antagonist is used in combination with another VEGF-C antagonist (i.e. the anti-neoplastic composition comprises a VEGF-C antagonist) such as, for example, a VEGF-C antibody, a VEGF-C variant, a soluble VEGF receptor molecule specific for VEGF-C, an aptamer capable of blocking VEGF-C or a VEGF-C receptor, a neutralizing VEGFR-3 antibody, a low molecular weight inhibitor of VEGFR-3 tyrosine kinases and any combinations thereof. Alternatively, or in addition, two or more antagonists of the same type may be co-administered to the subject. For example, two VEGF-C antibodies may be co-administered to the subject.

Other therapeutic agents useful for combination tumor therapy with the VEGF-C antagonist include antagonists of other factors that are involved in tumor growth, such as EGFR, ErbB2 (also known as HER2) ErbB3, ErbB4, or TNF. Sometimes, it may be beneficial also to administer one or more cytokines to the subject. In one example, the VEGF-C antagonist is co-administered with a growth inhibitory agent. For example, the growth inhibitory agent may be administered first, followed by the VEGF-C antagonist. However, simultaneous administration or administration of the VEGF-C antagonist first is also contemplated. Suitable dosages for the growth inhibitory agent are those presently used and may be lowered due to the combined action (synergy) of the growth inhibitory agent and VEGF-C antagonist.

In a preferred embodiment of the first or second aspect, effective amounts of a VEGF-C antagonist, such as a VEGF-C antibody, and one or more chemotherapeutic agents are administered in combination to a subject susceptible to, or diagnosed with, cancer (i.e. the anti-neoplastic composition comprises a chemotherapeutic agent). Any chemotherapeutic agent exhibiting anticancer activity can be used according to the present disclosure, including those defined above. The chemotherapeutic agent may be selected from the group consisting of docetaxel, 5-fluorouracil (5-FU), temozolomide (TMZ), gemcitabine, oxaliplatin, paclitaxel, carboplatin and irinotecan.

In a preferred embodiment, the method comprises administering in combination to the subject effective amounts of a VEGF-C antagonist, especially a VEGF-C antibody, and a standard chemotherapy for the cancer being or to be treated. It is envisaged that the method may be of particular benefit to treating a subject susceptible to or diagnosed with colorectal cancer, lung cancer, pancreatic cancer, prostate cancer, glioblastoma, kidney and renal cancer, breast cancer, liver cancer, non-Hodgkins lymphomas, ovarian cancer and brain cancers wherein effective amounts of a VEGF-C antagonist, especially an VEGF-C antibody, and a standard chemotherapy for the particular cancer are administered in combination to the subject. In particular, it is envisaged that the method may be of particular benefit to treating a subject susceptible to or diagnosed with colorectal cancer (especially metastatic CRC), lung cancer (especially non-small cell lung cancer, NSCLC), pancreatic cancer, prostate cancer, glioblastoma, kidney and renal cancer (especially metastatic renal cell carcinoma, RCC), pancreatric cancer, breast cancer (especially HER2 negative metastatic breast cancer), ovarian cancer and glioblastomas wherein effective amounts of a VEGF-C antagonist, especially an VEGF-C antibody, and a standard chemotherapy for the particular cancer are administered in combination to the subject.

There may be more than one “standard chemotherapy” or “standard chemotherapy regime” for a particular cancer type. For example, the “standard” therapy may vary depending on the stage of the cancer to be treated, whether the primary tumor has metastised or not, the age of the patient, variations in medical guidelines in different countries, etc. A standard chemotherapy for colorectal cancer is 5-FU. A standard chemotherapy for pancreatic cancer is gemcitabin. A standard chemotherapy for prostate cancer is docetaxel. A standard chemotherapy for glioblastoma is TMZ. The colorectal cancer, pancreatic cancer, prostate cancer or glioblastoma can be metastatic.

By way of example only, standard chemotherapy treatments for metastatic colorectal cancer are described herein below.

Colorectal cancer is the third most common cause of cancer mortality in the United States. It was estimated that approximately 129,000 new cases of colorectal cancer would be diagnosed and 56,000 deaths would occur due to colorectal cancer in the United States in 1999. Approximately 70% of colorectal cancer subjects present with disease that is potentially curable by surgical resection. However, the prognosis for the 30% who present with advanced or metastatic disease and for the 20% who relapse following resection is poor. The median survival for those with metastatic disease is 12-14 months.

The standard treatment for metastatic colorectal cancer in the United States has been until recently chemotherapy with 5-FU plus a biochemical modulator of 5-FU, leucovorin. The combination of 5-FU/leucovorin provides infrequent, transient shrinkage of colorectal tumors but has not been demonstrated to prolong survival compared with 5-FU alone, and 5-FU has not been demonstrated to prolong survival compared with an ineffective therapy plus best supportive care. The lack of a demonstrated survival benefit for 5-FU/leucovorin may be due in part to inadequately sized clinical trials. In a large randomized trial of subjects receiving adjuvant chemotherapy for respectable colorectal cancer, 5-FU/leucovorin demonstrated prolonged survival compared with lomustine (MeCCNU), vincristine, and 5-FU (MOF).

In the United States, 5-FU/leucovorin chemotherapy is commonly administered according to one of two schedules: the Mayo Clinic and Roswell Park regimens. The Mayo Clinic regimen consists of an intensive course of 5-FU plus low-dose leucovorin (425 mg/m² 5-FU plus 20 mg/m² leucovorin administered daily by intravenous (IV) push for 5 days, with courses repeated at 4- to 5-week intervals). The Roswell Park regimen consists of weekly 5-FU plus high-dose leucovorin (500-600 mg/m² 5-FU administered by IV push plus 500 mg/m² leucovorin administered as a 2-hour infusion weekly for 6 weeks, with courses repeated every 8 weeks). Clinical trials comparing the Mayo Clinic and Roswell Park regimens have not demonstrated a difference in efficacy, but have been underpowered to do so. The toxicity profiles of the two regimens are different, with the Mayo Clinic regimen resulting in more leukopenia and stomatitis and the Roswell Park regimen resulting in more frequent diarrhea. Subjects with newly diagnosed metastatic colorectal cancer receiving either regimen can expect a median time to disease progression of 4-5 months and a median survival of 12-14 months.

Recently, a new first-line therapy for metastatic colorectal cancer has emerged. Two randomized clinical trials, each with approximately 400 subjects, evaluated irinotecan in combination with 5-FU/leucovorin. In both studies, the combination of irinotecan/5-FU/leucovorin demonstrated statistically significant increases in survival (of 2.2 and 3.3 months), time to disease progression and response rates as compared with 5-FU/leucovorin alone. The benefits of irinotecan came at a price of increased toxicity: addition of irinotecan to 5-FU/leucovorin was associated with an increased incidence of National Cancer Institute Common Toxicity Criteria (NCI-CTC) Grade 3/4 diarrhea, Grade 3/4 vomiting, Grade 4 neutropenia, and asthenia compared with 5-FU/leucovorin alone. There is also evidence showing that single-agent irinotecan prolongs survival in the second-line setting. Two randomized studies have demonstrated that irinotecan prolongs survival in subjects who have progressed following 5-FU therapy. One study compared irinotecan to best supportive care and showed a 2.8-month prolongation of survival; the other study compared irinotecan with infusional 5-FU and showed a 2.2-month prolongation of survival. The question of whether irinotecan has more effect on survival in the first- or second-line setting has not been studied in a well-controlled fashion.

In a further embodiment of the first or second aspect, the anti-neoplastic composition comprises a chemotherapeutic agent and an anti-angiogenic agent. In one embodiment, the chemotherapeutic agent is selected from the group consisting of docetaxel, 5-fluorouracil (5-FU), temozolomide (TMZ), gemcitabine, oxaliplatin, paclitaxel, carboplatin and irinotecan and the anti-angiogenic agent is an antibody such as, for example, Bevacizumab.

In a preferred embodiment of the first or second aspect, the VEGF-C antagonist (for example a VEGF-C antibody) is administered in combination with a VEGF-A antagonist. A preferred VEFG-A antagonist is bevacizumab.

In a preferred embodiment of the first or second aspect, the VEGF-C antagonist (for example a VEGF-C antibody) is administered in combination with a VEGF-A antagonist (for example a VEFG-A antibody such as bevacizumab), optionally in combination with one or more additional anti-cancer therapeutic agents.

By way of example only, the following examples of potential combinations are described:

A VEGF-C antibody+a VEGF-A antibody+FOLFOX in the treatment of colorectal or lung cancer.

A VEGF-C antibody+a VEGF-A antibody+paclitaxel in the treatment of colorectal, lung or breast cancer.

A VEGF-C antibody+a VEGF-A antibody+paclitaxel+carboplatin in the treatment of lung cancer.

A VEGF-C antibody+a VEGF-A antibody+irinotecan in the treatment of colorectal cancer.

A VEGF-C antibody+a VEGF-A antibody+interferon in the treatment of renal cancer.

A VEGF-C antibody+a VEGF-A antibody in the treatment of glioblastoma.

The cancer may be late stage and/or metastatic in all of the above examples. In a preferred embodiment, the VEGF-A antibody is bevacizumab.

In one embodiment, the cancer to be treated is a resistant cancer. For example, the cancer to be treated is resistant to a VEGF-A antibody, especially bevacizumab. In a preferred embodiment, a subject loses or shows a reduced response over the course of a cancer therapy comprising a VEGF-antagonist, and in particular a VEGF-A antibody such as bevacizumab.

In a further embodiment of the first or second aspect, the cancer has become resistant to an anti-angiogenic VEGF-A antagonist, in which the subject receives a combination therapy comprising a VEGF-C antagonist, preferably a VEGF-C antibody, and an anti-neoplastic composition as described herein. Preferably, the cancer comprises a solid and/or vascularised tumor. In one embodiment, the standard of care therapy is revised by substituting the VEGF-A antagonist agent with a VEGF-C antagonist as described herein. In another embodiment, a VEGF-C antagonist as described herein is added to the standard of care therapy, i.e. the VEGF-A antagonist is maintained in the treatment regime.

The present inventors have also found that including a VEGF-C antagonist in a cancer treatment regime already including a VEGF-A antagonist, such as bevacizumab, at the commencement of the anti-cancer therapy (i.e. in patients who are naïve to the VEGF-A antagonist such as bevacizumab) or at a point in the anti-cancer therapy where no resistance to the VEGF-A antagonist is evident may provide significant benefits in terms of disease progression, tumor volume reduction, duration of survival, response rate and duration of response. In a further embodiment of the first or second aspect, the VEGF-C antagonist is administered in combination with an anti-neoplastic composition comprising at least one chemotherapeutic agent and at least one VEGF-A antagonist. For example, a VEGF-C antibody is suitably administered in combination with a VEGF-A antibody and a chemotherapeutic agent.

Dosage and Administration

It will be appreciated by one of skill in the medical arts that the exact manner of administering to a subject a therapeutically effective amount of an anti-cancer agents (e.g. the VEGF-C antagonist and the anti-neoplastic composition) following a diagnosis of a patient's likely responsiveness to the anti-cancer agent will be at the discretion of the attending physician. The mode of administration, including dosage, combination with other agents, timing and frequency of administration, and the like, may be affected by the diagnosis of a patient's likely responsiveness to such anti-cancer agent, as well as the patient's condition and history. Even patients diagnosed with a disorder who are predicted to be relatively insensitive to the anti-cancer agent may still benefit from treatment therewith, particularly in combination with other agents.

The therapeutic compositions comprising the anti-cancer agents will be formulated, dosed, and administered in a fashion consistent with good medical practice. Factors for consideration in this context include the particular type of disorder being treated, the particular mammal being treated, the clinical condition of the individual patient, the site of delivery of the agent, possible side-effects, the type of VEGF-C antagonist, the method of administration, the scheduling of administration, and other factors known to medical practitioners. The effective amount of the anti-cancer agents to be administered will be governed by such considerations.

The VEGF-C antagonist (e.g. antibodies) and anti-neoplastic composition, including therapeutic agents, may be administered to a subject by any suitable method including parenteral (e.g. intravenous (IV) administration), intramuscular, intraperitoneal, intracerobrospinal, subcutaneous (SC), intra-articular, intrasynovial, intrathecal, oral, topical and inhalation routes (e.g. intrapulmonary). Parenteral infusions include intramuscular, IV, intraarterial, intraperitoneal and SC administration. IV or SC administration of the VEGF-C antagonist (e.g. antibody) is preferred. Most preferably, the VEGF-C antagonist is administered by IV infusion.

The term “intravenous infusion” refers to introduction of a drug into the vein of an animal or human subject over a period of time greater than approximately 5 minutes, preferably between approximately 30 to 90 minutes, although, intravenous infusion is alternatively administered for 10 hours or less. The subject may receive the infusion continuously over a period of time or alternatively as a bolus or push. The term “intravenous bolus” or “intravenous push” refers to drug administration into a vein of an animal or human such that the body receives the drug in approximately 15 minutes or less, or in approximately 5 minutes or less.

The term “subcutaneous administration” refers to introduction of a drug under the skin of an animal or human subject, preferably within a pocket between the skin and underlying tissue, by relatively slow, sustained delivery from a drug receptacle. The pocket may be created by pinching or drawing the skin up and away from underlying tissue.

The term “subcutaneous infusion” refers to introduction of a drug under the skin of an animal or human subject, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human subject, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human subject, where bolus drug delivery is less than approximately 15 minutes, less than 5 minutes, or less than 60 seconds. Administration is preferably within a pocket between the skin and underlying tissue, where the pocket is created, for example, by pinching or drawing the skin up and away from underlying tissue.

The combined administration includes co-administration, using separate formulations or a single pharmaceutical formulation, and consecutive administration in either order, wherein preferably there is a time period while both (or all) active agents simultaneously exert their biological activities.

Aside from administration of anti-cancer agents to the patient by traditional routes as noted above, the present invention includes administration by gene therapy. Such administration of nucleic acids encoding the anti-cancer agent is encompassed by the expression “administering an effective amount of an anti-cancer agent”. See, for example, WO 1996/07321 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antagonist is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent specific for the target cells, such as an antibody specific for a cell-surface membrane protein on the target cell, a ligand for a receptor on the target cell, etc. Where liposomes are employed, proteins that bind to a cell-surface membrane protein associated with endocytosis may be used for targeting and/or to facilitate uptake, e.g. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., PNAS USA 87:3410-3414 (1990). Gene-marking and gene therapy protocols are described, for example, in Anderson et al., Science 256:808-813 (1992) and WO 1993/25673.

The pharmaceutical formulation containing the VEGF-C antagonist (e.g., an VEGF-C antibody) may also comprise a therapeutically effective amount of the anti-neoplastic agent such as, for example, a chemotherapeutic agent, a growth inhibitory agent, a cytotoxic agent, a cytokine, an anti-hormonal agent, an antiangiogenic agent, an anti-lymphangiogenic agent, and combinations thereof. Such molecules are suitably present in combination in amounts that are effective for the purpose intended.

In one embodiment, the VEGF-C antagonist is a VEGF-C antibody and the anti-neoplastic agent is a therapeutic antibody other than a VEGF-C antibody, such as a VEGF-A antibody, e.g. bevacizumab. Other therapeutic antibodies that may be employed as anti-neoplastic agents include antibodies that bind a cancer cell surface marker. For example, the therapeutic antibody may be HER2 antibody, trastuzumab (e.g., Herceptin®, Genentech, Inc., South San Francisco, Calif.) or HER2 antibody, pertuzumab (Onmitarg™, Genentech, Inc., South San Francisco, Calif., see U.S. Pat. No. 6,949,245).

Other therapeutic regimens in accordance with this invention may include administration of the VEGF-C antagonist and, including without limitation, radiation therapy and/or bone marrow and peripheral blood transplants, and/or a cytotoxic agent, a chemotherapeutic agent, or a growth inhibitory agent. In one embodiment, a chemotherapeutic agent is an agent or a combination of agents such as, for example, CHOP (an abbreviation for the combined therapy of cyclophosphamide, hydroxydaunorubicin (adriamycin; doxorubincin), vincristine (ONCOVIN™) and prednisolone); CVP (combination therapy of cyclophosphamide, vincristine and prednisolone—used, e.g. for low grade non-Hodgkin's lymphoma); COP (combination therapy of cyclophosphamide, vincristine and prednisolone—used, e.g. to treat leukemia); FOLFOX (an abbreviation for a treatment regimen with oxaliplatin (ELOXATIN™) combined with 5-FU and leucovovin); or immunotherapeutics such as anti-PSCA, anti-HER2 (e.g., HERCEPTIN®, OMNITARG™). The combination therapy may be administered as a simultaneous or sequential regimen. Additional chemotherapeutic agents include the cytotoxic agents useful as antibody drug conjugates, such as, for example, maytansinoids (DMI, for example) and the auristatins MMAE and MMAF. Drugs to counteract the side effects of, for example, the chemotherapeutic agents, may also be coadministered. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the VEGF-C antagonist and other chemotherapeutic agents or treatments.

The combination therapy may provide “synergy” and prove “synergistic”, i.e. the effect achieved when the active ingredients used together is greater than the sum of the effects that results from using the compounds separately. A synergistic effect may be attained when the active ingredients are: (I) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. An effective dosage of each active ingredient may be administered sequentially, i.e. serially, or effective dosages of two or more active ingredients may be administered together.

As will be understood by those of ordinary skill in the art, the appropriate doses of chemotherapeutic agents will be generally around those already employed in clinical therapies wherein the chemotherapeutics are administered alone or in combination with other chemotherapeutics. Variation in dosage will likely occur depending on the condition being treated. The physician administering treatment will be able to determine the appropriate dose for the individual subject.

For the prevention or treatment of disease, the appropriate dosage of VEGF-C antagonist will depend on the type of disease to be treated, as defined above, the severity and course of the disease, whether the antibody is administered for preventive or therapeutic purposes, previous therapy, the subject's clinical history and response to the antibody, and the discretion of the attending physician. Administration targeting one organ or tissue, for example, may necessitate delivery in a manner different from that to another organ or tissue. The antibody is suitably administered to the subject at one time or over a series of treatments. In a combination therapy regimen, the compositions of the present disclosure are administered in a therapeutically effective or synergistic amount.

Normal dosage amounts may vary from about 1 ng/kg to about 1 g/kg or more of mammal body weight per day. For example, dosage amounts may be from about 10 ng/kg/d to about 100 mg/kg/d, from about 100 ng/kg/d to about 10 mg/kg/d, from about 1 μg/kg/d to about 10 mg/kg/day, or about 10 μg/kg/d, about 50 μg/kg/d, about 100 μg/kg/d, about 500 μg/kg/d, about 1 mg/kg/d, about 2.5 mg/kg/d, about 5 mg/kg/d, about 10 mg/kg/d, about 20 mg/kg/d, about 30 mg/kg/d, about 40 mg/kg/d, about 50 mg/kg/d, about 60 mg/kg/d, about 70 mg/kg/d, about 80 mg/kg/d, about 90 mg/kg/d, about 100 mg/kg/d, about 250 mg/kg/d, or about 500 mg/kg/d, depending upon the route of administration.

Depending on the type and severity of the disease, about 1 μg/kg to 50 mg/kg (e.g. 0.1-20 mg/kg) of antagonist (e.g. VEGF-C antibody) is an initial candidate dosage for administration to the subject, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful.

The antagonist (e.g. antibody) may be administered every two to three weeks, at a dose ranged from about 5 mg/kg to about 15 mg/kg. More preferably, such dosing regimen is used in combination with a chemotherapy regimen as the first line therapy for treating cancer, e.g. metastatic colorectal cancer. The chemotherapy regimen may involve the traditional high-dose intermittent administration. The chemotherapeutic agents may be administered using smaller and more frequent doses without scheduled breaks (“metronomic chemotherapy”). The progress of the therapy of the invention is easily monitored by conventional techniques and assays.

Efficacy of the Treatment

The main advantage of the treatment of the present disclosure is the ability of producing marked anti-cancer effects in a human subject without causing significant toxicities or adverse effects, so that the subject benefits from the treatment overall. The efficacy of the treatment can be measured by various endpoints commonly used in evaluating cancer treatments, including but not limited to, tumor regression, tumor weight or size shrinkage, time to progression, duration of survival, progression free survival, overall response rate, duration of response, and quality of life. Because the anti-angiogenic agents target the tumor vasculature and not necessarily the neoplastic cells themselves, they represent a unique class of anticancer drugs, and therefore may require unique measures and definitions of clinical responses to drugs. For example, tumor shrinkage of greater than 50% in a 2-dimensional analysis is the standard cut-off for declaring a response. However, the VEGF-C antagonist may cause inhibition of metastatic spread without shrinkage of the primary tumor, or may simply exert a tumoristatic effect. Accordingly, novel approaches to determining efficacy of an anti-angiogenic therapy should be employed, including for example, measurement of plasma or urinary markers of angiogenesis and measurement of response through radiological imaging.

Disclosed herein is a method for increasing the duration of survival of a human subject susceptible to or diagnosed as having a cancer, comprising administering in combination to the subject effective amounts of a VEGF-C antagonist and an anti-neoplastic composition. The anti-neoplastic composition may comprise at least one chemotherapeutic agent, wherein administration of the VEGF-C antagonist and the anti-neoplastic composition effectively increases the duration of survival.

For example, a subject group treated with the VEGF-C antagonist combined with the anti-neoplastic composition comprising at least two, optionally three, therapeutic agents (e.g. chemotherapeutic agents) may have a median duration of survival that is at least about 2 months, preferably between about 2 and about 5 months, longer than that of the subject group treated with the same chemotherapeutic cocktail alone, said increase being statistically significant. A combination treatment of the VEGF-C antagonist and the anti-neoplastic composition comprising one or more therapeutic agents (e.g. chemotherapeutic agents) may significantly reduce the risk of death by at least about 30% (i.e., a stratified HR of about 0.70), or by at least about 35% (i.e., a stratified HR of about 0.65), when compared to a chemotherapy alone.

Also disclosed is a method for increasing the progression free survival of a human subject susceptible to or diagnosed as having a cancer, comprising administering in combination to the subject effective amounts of a VEGF-C antagonist and an anti-neoplastic composition. The anti-neoplastic composition may comprise at least one chemotherapeutic agent, wherein administration of the VEGF-C antagonist and the anti-neoplastic composition effectively increases the duration of progression free survival.

For example, the combination treatment of the disclosure using a VEGF-C antagonist and one or more chemotherapeutic agents significantly may increase progression free survival by at least about 2 months, preferably by about 2 to about 5 months, when compared to a treatment with chemotherapy alone.

Also disclosed is a method for treating a group of subjects susceptible to or diagnosed as having a cancer, comprising administering in combination to the group effective amounts of a VEGF-C antagonist and an anti-neoplastic composition. The anti-neoplastic composition may comprise at least one chemotherapeutic agent, wherein administration of the VEGF-C antagonist and the anti-neoplastic composition effectively increases the response rate in the group of subjects. For example, the combination treatment may significantly increase the response rate in the treated subject group compared to the group treated with chemotherapy alone, said increase having a Chi-square p-value of less than 0.005.

Also disclosed is a method for increasing the duration of response in a human subject or a group of human subjects susceptible to or diagnosed as having a cancer, comprising administering in combination to the subject or group effective amounts of an VEGF-C antagonist and an anti-neoplastic composition. The anti-neoplastic composition may comprise at least one chemotherapeutic agent, wherein administration of the VEGF-C antagonist and the anti-neoplastic composition effectively increases the duration of response. For example, a combination treatment using a VEGF-C antagonist and one or more chemotherapeutic agents, a statistically significant increase of at least 2 months in duration of response may be obtainable.

Also disclosed is a method of treating a human subject or a group of human subjects having metastatic colorectal cancer, prostate cancer, pancreatic cancer or glioblastoma, comprising administering in combination to the subject or group effective amounts of a VEGF-C antagonist and an anti-neoplastic composition, wherein said anti-neoplastic composition comprises at least one chemotherapeutic agent, wherein administration of the VEGF-C antagonist and the anti-neoplastic composition results in statistically significant and clinically meaningful improvement of the treated subject or group as measured by the duration of survival, progression free survival, response rate or duration of response.

Safety of the Treatment

Disclosed herein are methods of effectively treating cancers without significant adverse effects to the subject. Combination therapy of the disclosure using VEGF-C antagonists combined with a chemotherapy cocktail comprising at least two, or three, chemotherapeutic agents does not significantly increase incident occurrences of adverse events, when compared with the chemotherapy alone. Thus, the treatment of the present disclosure unexpectedly contains side effects at an acceptable level, and at the same time significantly improves anti-cancer efficacy.

Articles of Manufacture (Kits)

An article of manufacture containing materials useful for the treatment of the disorders or cancers described above is also disclosed. The article of manufacture may comprise a container, a label and a package insert. Suitable containers include, for example, bottles, vials, syringes, etc. The containers may be formed from a variety of materials such as glass or plastic. The container holds a composition which is effective for treating the disorder or cancer and may have a sterile access port (for example the container may be an intravenous solution bag or a vial having a stopper pierceable by a hypodermic injection needle). At least one active agent in the composition is a VEGF-C antagonist (e.g. a VEGF-C antibody). The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, and syringes. In addition, the article of manufacture comprises a package insert with instructions for use, including for example a warning that the composition is not to be used in combination with an anthracycline-type chemotherapeutic agent, e.g. doxorubicin, or epirubicin, or instructing the user of the composition to administer the VEGF-C antagonist and an anti-neoplastic composition to a subject.

The article of manufacture may be in the form of a kit. A kit for treating cancer in a subject may comprise a VEGF-C antagonist and an anti-neoplastic composition. In another embodiment, the kit for treating cancer in a subject may comprise a VEGF-C antagonist and instructions for using the VEGF-C antagonist in combination with an anti-neoplastic composition. The anti-neoplastic agent may comprise at least one chemotherapeutic agent, e.g. a VEGF-A antibody, for treating cancer in a subject.

Pharmaceutical Formulations

Therapeutic formulations of the antagonists used in accordance with the present disclosure are prepared for storage by mixing an antibody having the desired degree of purity with optional pharmaceutically acceptable carriers, excipients or stabilizers in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as ethylenediaminetetraacetic acid (EDTA); sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized VEGF-A antibody formulations are described in WO 97/04801.

The formulation herein may also contain more than one active compound as necessary for the particular indication being treated, preferably those with complementary activities that do not adversely affect each other. For example, it may be desirable to further provide antibodies which bind to EGFR, VEGF-A, a VEGFR, or ErbB2 (e.g., Herceptin®) in the one formulation. Alternatively, or in addition, the composition may comprise a cytotoxic agent, cytokine, growth inhibitory agent and/or small molecule VEGFR antagonist. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The following examples are intended merely to illustrate the practice of the present invention and are not provided by way of limitation. The disclosures of all patent and scientific literatures cited herein are expressly incorporated in their entirety by reference. However, it is to be understood that, if any prior art publication is referred to herein, such reference does not constitute an admission that the publication forms a part of the common general knowledge in the art in Australia or any other country.

### EXAMPLES

### Example 1

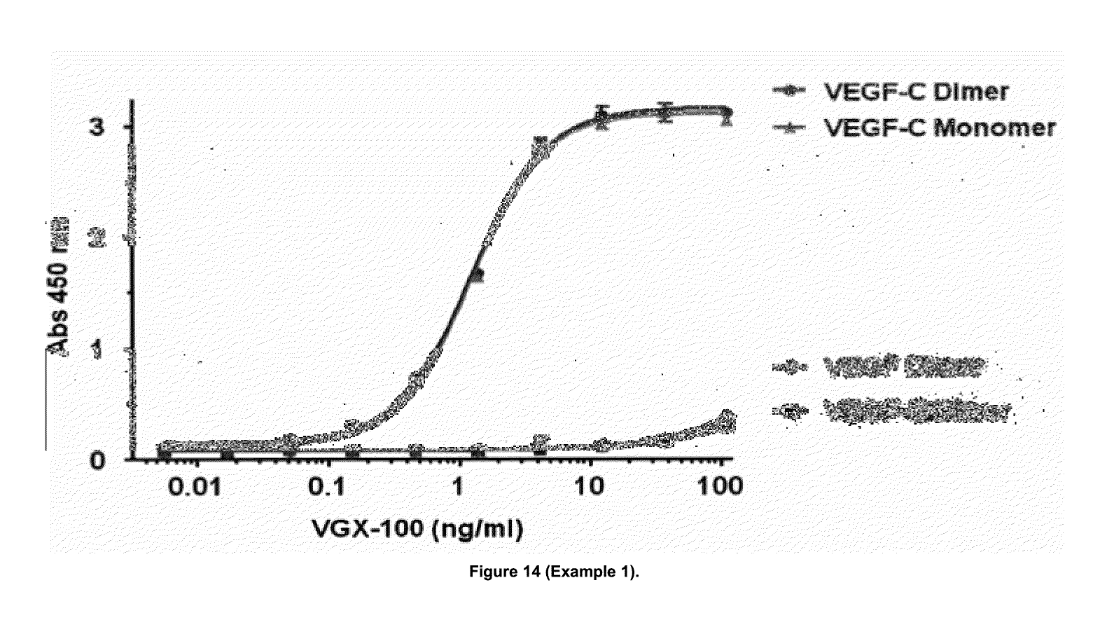

VEGF-C antibody VGX-100 was assessed by a direct binding ELISA using VEGF-C, VEGF-D or VEGF (R&D Systems) as capture antigens and bound VGX-100 detected with rabbit anti-human IgG-HRP (Abcam). The results are presented in FIG. 14. VGX-100 selectively recognized and bound to VEGF-C by ELISA with a K_{D} 1.8 nM (BiaCore).

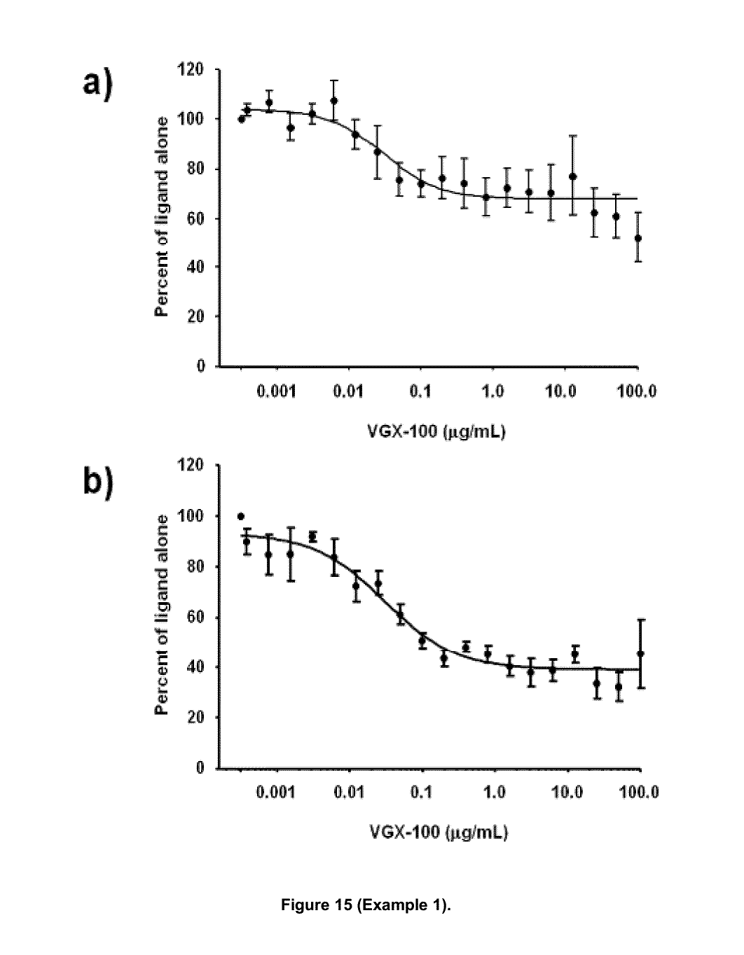

Bioassays to measure the binding of VEGF-C to the extracellular domain of VEGFR-2 or VEGFR-3 were performed with BA/F3-VEGFR-2 or BA/F3-VEGFR-3/EpoR cells. The response to ligands and VGX-100 was measured by [³H] thymidine incorporation following exposure for 48 hrs. The results are presented in FIG. 15. VGX-100 blocked VEGF-C binding to (a) VEGFR-2 and (b) VEGFR-3 in Ba/F3 bioassays.

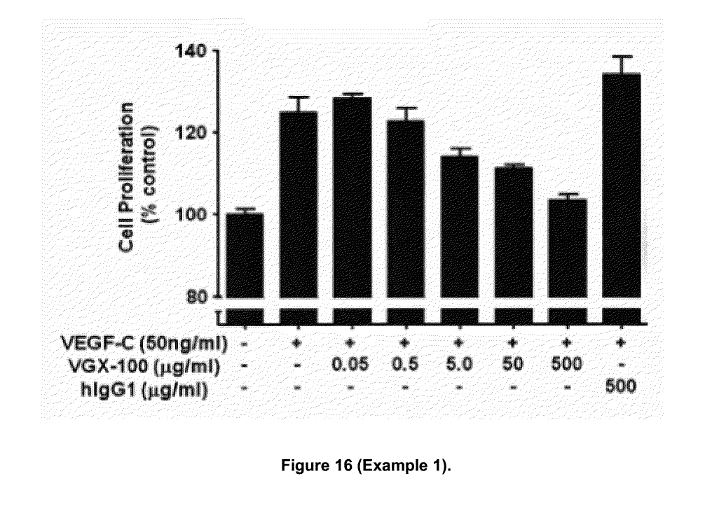

HUVEC proliferation assays were conducted for 48 hrs and cell number measured with WST-1 reagent (Roche). The results are shown in FIG. 16. VGX-100 inhibited VEGF-C stimulated HUVEC proliferation.

### Example 2

### Prostate Cancer (PC-3) Single Therapy

To initiate the study, 5×10⁶ PC-3 cells suspended in 100 μl of a mixture of medium/Matrigel (1:1) were subcutaneously implanted to the nude mice in the right flank region. Cells were implanted into 80 mice. Animals were monitored for tumor growth daily after cell implantation. When tumor volumes reached 80-100 mm³, mice were randomized into 5 groups of 10 mice each using only mice having tumor volumes closest to the mean value. Mice with tumor volumes too big or too small were excluded from the study. Tumor volumes were measured using the formula V=L×W×H×π/6, where L and W represent the longer and shorter diameters of the tumor and H represents the height of the tumor. The treatment with drugs began on the day after randomization. Vehicle control, test antibody and the VEGF-A antibody bevacizumab were administered by intraperitoneal injection in volumes ranging between 68-108 μl per dose. Bevacizumab was supplied as drug in aqueous solution at a concentration of 25 mg/ml and diluted 10 fold with PBS. The treatment was carried out twice weekly for 21 days. Throughout the entire study, tumor volumes were measured twice weekly and body weights once weekly. Animals were observed for possible toxic effects from the drug treatment. Unscheduled sacrifices were performed if tumor volumes reached >1,500 mm³, animals lost >25% of their original body weights, tumor ulceration appeared, or mice became moribund. At the end of the experiment, statistical analysis was performed on the tumor growth rate of each treatment group versus that of the control. At the termination of the animals, tumors were removed, embedded in OCT, and stored at −80° C. for IHC analysis. Gross examination was conducted on liver, lung, spleen, kidneys, testes, and prostate. If no abnormal observation was detected on these organs, the tissues were discarded.

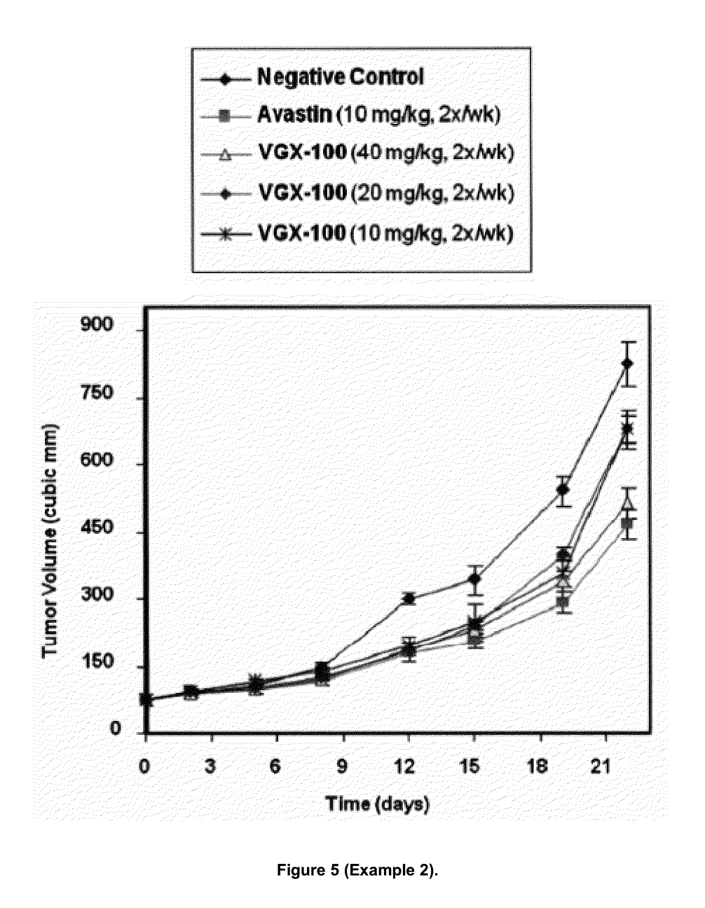

VEGF-C antibody VGX-100 (40 mg/kg) exhibited significant single-agent activity in a subcutaneous PC-3 xenograft tumor model, with equivalent efficacy to VEGF-A antibody bevacizumab (10 mg/kg) (FIG. 5).

### Example 3

### Combination Therapy in a Prostate Cancer (PC-3) Xenograft Model

This experiment evaluated the anticancer efficacy of the VEGF-C antibody VGX-100 alone and in combination with the VEGF-A antibody bevacizumab, the chemotherapeutic agent docetaxel, and bevacizumab+docetaxel against established PC-3 human prostate carcinoma xenografts in male nu/nu mice. Specifically, the following treatments were evaluated: (i) VGX-100+bevacizumab; (ii) VGX-100+docetaxel; (iii) VGX-100+docetaxel+bevacizumab; (iv) docetaxel; and (v) docetaxel+bevacizumab. VGX-100 and bevacizumab were administered at 10 mg/kg intraperitonealy every three days for two injections, followed by three days of rest for the duration of the experiment (twice each week, 3 days apart). Docetaxel was administered intravenously at 10 mg/kg weekly for three injections.

Materials and Methods

Chemicals

HIgG1 Isotype control-humanised antibody (6.4-12.36 mg/ml) and VGX-100 (5.53 and 5.75 mg/ml) were provided as frozen colorless stock solutions in Dulbecco's phosphate buffered saline (DPBS) and stored from light at −80° C. The stock solutions were thawed and stored at 4° C. when treatment began. The dosing solutions (40 mg/kg in 0.2 ml/20 g body weight) were prepared by diluting the stock solution with DPBS. The resulting formulations were clear with pH values ranging from 7.02-7.21. The dosing vials were kept on wet ice during dosing, gently pipetted and not aspirated through a needle except during administration. Dosing solutions were prepared weekly in DPBS and stored protected from light at 4° C. while not in use.

Bevacizumab (lot: 773313, 25 mg/ml) was obtained from McKesson Specialty Care Solutions as a colorless solution and stored at 4° C. Bevacizumab was prepared immediately prior to dosing by diluting the stock solution with water (pH=6.5).

Docetaxel (lot no. D9A095) was manufactured by Sanofi-Aventis and obtained from McKesson as a pale yellow solution in 50% EtOH/50% Tween80. It was stored protected from light at 4° C. Docetaxel was diluted as directed on the package insert with the provided diluent (sterile water) to produce a clear and colorless 10 mg/ml stock solution. On each treatment day, a fresh 10 mg/ml stock was prepared and further diluted with water to achieve the appropriate concentration. The resulting solution was clear and colorless with a pH of 7.16.

All test articles were administered within two hours of preparation. For groups receiving combination therapy, the drugs were administered in the order presented in Table 1 and within 30 minutes of one another.

Animals and Husbandry

Male NCR (NCRNU-M) athymic mice were obtained from Taconic. They were 6-7 weeks old on Day 1 of the experiment. The mice were fed irradiated Rodent Diet 5053 (LabDiet™) and water ad libitum. The mice were housed in static cages with Bed-O'Cobs™ bedding inside Biobubble® Clean Rooms that provide H.E.P.A filtered air into the bubble environment at 100 complete air changes per hour. All treatments, body weight determinations, and tumor measurements were carried out in the bubble environment. The environment was controlled to a temperature range of 21.1±1.1° C. (70±2° F.) and a humidity range of 30-70%.

Test mice were implanted subcutaneously on Day 0. All mice were observed for clinical signs at least once daily. Mice with tumors in excess of 2 g or with ulcerated tumors were euthanized, as were those found in obvious distress or in a moribund condition. All procedures carried out in this experiment were conducted in compliance with all the laws, regulations and guidelines of the National Institutes of Health (NIH) and with the approval of Discovery and Imaging Services, Ann Arbor's (DIS-AA) Animal Care and Use Committee.

Cell Preparation

PC-3 cells were obtained from ATCC and expanded using Ham's F-12 media supplemented with 10% fetal bovine serum and 1% penicillin-streptomycin-glutamine in 5% CO₂ atmosphere at 37° C. When expansion was complete, the cells were collected using Trypsin (Cell Gro®), the trypsin neutralized, and the cells pooled for implantation. The PC-3 (Passage 10) cell suspension was counted using trypan blue exclusion with a hemacytometer. The cell suspension was reduced to a pellet using an Eppendorf 5810R centrifuge at 1500 rpm (300×g) for 5 minutes at 4° C. A 2.5×10⁷ cells/ml suspension was prepared in 50% serum free media and 50% Matrigel®. Pre-injection viability was 97.7%. Test animals were implanted subcutaneously on Day 0 with 5×10⁶ cells/animal (0.2 ml) using a 27-gauge needle and syringe. The cell suspension was maintained on wet ice to minimize the loss of viability and inverted frequently to maintain a uniform cell suspension during the inoculation procedure.

Treatment

Treatments began on Day 8, when the mean estimated tumor mass for all groups in the experiment was 129 mg (range of group means, 123-134 mg). All animals weighed ≧19.2 g at the initiation of therapy. Mean group body weights at first treatment were matched (range of group means, 21.5-22.8 g). All animals were dosed according to individual body weight on the day of treatment (0.2 ml/20 g).

Measurements and Endpoints

Body weights and tumor measurements were recorded thrice weekly. Tumor burden (mg) was estimated from caliper measurements by the formula for the volume of a prolate ellipsoid assuming unit density as: Tumor burden (mg)=(L×W²)/2, where L and W are the respective orthogonal tumor length and width measurements (mm). The primary endpoints used to evaluate efficacy were: tumor growth delay, % T/C (defined as the median tumor mass of the Treated Group divided by the median tumor mass of the Control Group x 100), complete and partial tumor response, and the number of tumor-free survivors at the end of the study, where T and C are the median times in days required for the treatment and control group tumors, respectively, to grow to a selected evaluation size, 750 mg. Tumor growth delay for this experiment was expressed as a T-C value. In this experiment, % T/C was evaluated when the median Control reached 1 g.

A complete response or regression (CR) is defined as a decrease in tumor mass to an undetectable size (<50 mg). As used herein, “complete Regression (CR)” is credited to an animal if the tumor burden is reduced to an immeasurable volume at any point after the first treatment. Any tumor measurement less than 3 mm is recorded as “0”. This is in keeping with the convention of the National Cancer Institute and reflects the inherent and unacceptably high mechanical error in such measurements in addition to the uncertain biology of what is measured at those small sizes.

A partial response or regression (PR) is defined as a ≧50% decrease in tumor mass or burden from that at first treatment. PRs are exclusive of CRs, as are Tumor-Free Survivors (TFS).

“% Tumor Free Survivors (TFS)” refers to any animal with no measurable evidence of disease on the last day of the experiment. This value is exclusive of CRs.

“Tumor Doubling Time (Td)” refers to the growth rate of the tumor expressed as the volume doubling time (days) and is calculated from a log-linear least squares regression of the exponential portion of the tumor growth curve.

“Time to Evaluation Size” refers to the time (days) it takes a tumor to reach the specified Evaluation Size and is calculated from a log-linear least squares best fit of tumor burden versus time for the exponential portion of the final (post-treatment) tumor growth curve. This value is calculated for every animal in the experiment. The group medians are then used to calculate the Tumor Growth Delay.

“Time to Fold Growth End Point” refers to the occasional (usually when initial mean tumor burdens across all groups are not well-matched) situation when it is advantageous to display efficacy parameters in terms of fold growth, where the selected endpoint is the time it takes to reach a selected multiple of initial tumor burden. This increases the probability of uncovering a statistically significant therapeutic effect by eliminating the confounding effects of disparity in initial tumor sizes. The “Time to Fold Growth End Point” is calculated as described for Time to Evaluation Size from the fold growth data.

“Tumor Burden at Last Rx” refers to the tumor burden on the last day of treatment. This value is calculated from a log-linear least squares best fit of tumor burden versus time for the exponential portion of the final (post-treatment) tumor growth curve.

“Tumor Growth Delay (T-C)” refers to the difference between the median times it takes the treated group and the control group (the first group in Table 2) to reach the stated evaluation size. This is calculated from the median times to evaluation size for each animal in the group, not from interpolation of the median growth curve.

“Apparent Net Tumor Cell Kill” refers to the net change in tumor burden (measured in logs) between the first and last treatments. By interpolation, the log-linear regression line for each animal is used to calculate the tumor weight (TW) at the start of treatment, and the TW at the end of treatment, for control and treatment groups, respectively. For each treatment group, the median TW (end of treatment) is subtracted from the median control TW (start of treatment) to calculate the apparent net cell kill (in log units).

TABLE 1
Group Summary

| | | | | | | Efficacy | | | | |
|---|---|---|---|---|---|---|---|---|---|---|
| | | Dose | | | % T/C | Tumor Growth | Apparent Net Tumor | | | |
| Group | Treatment | mg/kg/inj | Schedule | Route | Day 22 | Delay (days) | Cell Kill (logs) | % CR | % PR | % TFS |
| 1 | Negative isotype | 40 | (Q3Dx2; 3) x22 | IP | 100 | NA | NA | 0 | 0 | 0 |
| | control | | | | | | | | | |
| 2 | Bevacizumab | 10 | (Q3Dx2; 3) x21 | IP | 70 | 5.6 | −4.91 | 0 | 0 | 0 |
| 3 | VGX-100 | 40 | (Q3Dx2; 3) x9 | IP | 87 | 2.2 | −2.85 | 0 | 0 | 0 |
| 4 | Docetaxel | 10 | Q7Dx3 | IV | 8 | 56.5 | 1.13 | 100 | 0 | 20 |
| 5 | Bevacizumab + | 10 + 10 | (Q3Dx2; 3) x22 + | IP + IV | 8 | 76.7 | −2.20 | 70 | 0 | 0 |
| | docetaxel | | Q7Dx3 | | | | | | | |
| 6 | VGX-100 + | 40 + 10 | (Q3Dx2; 3) x22 + | IP + IV | 8 | 110.3 | −2.06 | 100 | 0 | 20 |
| | docetaxel | | Q7Dx3 | | | | | | | |
| 7 | VGX-100 + | 40 + 10 | (Q3Dx2; 3) x17 + | IP + IP | 76 | 8.2 | −4.69 | 0 | 0 | 0 |
| | bevacizumab | | (Q3Dx2; 3)x17 | | | | | | | |
| 8 | VGX-100 + | 40 + 10 + | (Q3Dx2; 3) x22 + | IP + IP + | 7 | >140 | NA | 100 | 0 | 40 |
| | bevacizumab + | 10 | (Q3Dx2; 3)x22 + | IV | | | | | | |
| | docetaxel | | Q7Dx3 | | | | | | | |

TABLE 2

| | | | Growth Endpoints | | | | |
|---|---|---|---|---|---|---|---|
| | | Time to | Time to | | | | |
| | | evaluation | fold growth | Complete | Partial | Tumor free | Animal |
| Animal | Td (days) | size (days) | EP (days) | regression | regression | survivor | endpoint |

Group 1: Negative isotype Control Dose: 40

| | | | | | | | |
|---|---|---|---|---|---|---|---|
| 1 | 7.7 | 23.3 | 20.3 | no | no | no | TD |
| 2 | 5.9 | 19.0 | 18.2 | no | no | no | TD |
| 2 | 6.4 | 24.5 | 19.4 | no | no | no | TD |
| 4 | 10.4 | 36.7 | 30.7 | no | no | no | TD |
| 5 | 4.8 | 20.0 | 17.3 | no | no | no | TD |
| 6 | 4.7 | 17.6 | 14.2 | no | no | no | TD |
| 7 | 6.3 | 19.9 | 17.5 | no | no | no | TD |
| 8 | 9.6 | 31.0 | 23.4 | no | no | no | TD |
| 9 | 5.7 | 18.3 | 17.6 | no | no | no | TD |
| 10 | 5.6 | 19.5 | 16.3 | no | no | no | TD |
| Mean | 6.7 | 23.0 | 19.5 | Total | Total | Total | Total |
| SD | 1.9 | 6.3 | 4.7 | CR | PR | TFS | Excluded |
| Median | 6.1 | 20.0 | 17.9 | 0 | 0 | 0 | 0 |

Group 2: Bevacizumab Dose: 10

| | | | | | | | |
|---|---|---|---|---|---|---|---|
| 1 | 16.9 | 36.3 | 26.6 | no | no | no | TD |
| 2 | 7.4 | 24.4 | 20.2 | no | no | no | TD |
| 2 | 7.5 | 25.8 | 21.4 | no | no | no | TD |
| 4 | 13.7 | 33.0 | 22.1 | no | no | no | TD |
| 5 | 16.8 | 30.5 | 20.9 | no | no | no | TD |
| 6 | 10.6 | 25.2 | 21.2 | no | no | no | TD |
| 7 | 8.6 | 25.4 | 20.3 | no | no | no | TD |
| 8 | 27.8 | 44.3 | 28.3 | no | no | no | TD |
| 9 | 4.8 | 18.0 | 15.3 | no | no | no | TD |
| 10 | 8.4 | 22.7 | 17.8 | no | no | no | TD |
| Mean | 12.2 | 28.6 | 21.4 | Total | Total | Total | Total |
| SD | 6.8 | 7.6 | 3.8 | CR | PR | TFS | Excluded |
| Median | 9.6 | 25.6 | 21.0 | 0 | 0 | 0 | 0 |

Group 3: VGX-100 Dose: 40

| | | | | | | | |
|---|---|---|---|---|---|---|---|
| 1 | 16.6 | 39.5 | 27.4 | no | no | no | TD |
| 2 | 5.1 | 22.8 | 20.9 | no | no | no | TD |
| 3 | 6.0 | 19.9 | 16.4 | no | no | no | TD |
| 4 | 6.1 | 22.3 | 21.5 | no | no | no | TD |
| 5 | 14.1 | 33.5 | 28.2 | no | no | no | TD |
| 6 | 5.7 | 18.5 | 13.9 | no | no | no | TD |
| 7 | 12.3 | 31.1 | 21.3 | no | no | no | TD |
| 8 | 6.3 | 19.4 | 15.8 | no | no | no | TD |
| 9 | 7.1 | 22.0 | 17.9 | no | no | no | TD |
| 10 | 3.8 | 22.2 | 20.7 | no | no | no | TD |
| Mean | 8.3 | 25.1 | 20.4 | Total | Total | Total | Total |
| SD | 4.4 | 7.1 | 4.7 | CR | PR | TFS | Excluded |
| Median | 6.2 | 22.2 | 20.8 | 0 | 0 | 0 | 0 |

Group 4: Docetaxel Dose: 10

| | | | | | | | |
|---|---|---|---|---|---|---|---|
| 1 | 5.1 | 81.0 | 78.1 | yes | no | no | TD |
| 2 | NA | >160 | >160 | yes | no | yes | TFS |
| 3 | 8.5 | 62.3 | 57.4 | yes | no | no | TD |
| 4 | 9.4 | 89.1 | 81.6 | yes | no | no | TD |
| 5 | 9.1 | 72.1 | 65.4 | yes | no | no | TD |
| 6 | 13.2 | 69.0 | 58.5 | yes | no | no | TD |
| 7 | 6.5 | 62.5 | 60.0 | yes | no | no | TD |
| 8 | 13.4 | 81.0 | 73.3 | yes | no | no | TD |
| 9 | NA | >160 | >160 | yes | no | yes | TFS |
| 10 | 5.0 | 66.5 | 65.9 | yes | no | no | TD |
| Mean | 8.8 | NA | 86.0 | Total | Total | Total | Total |
| SD | 3.3 | NA | 39.8 | CR | PR | TFS | Excluded |
| Median | 8.8 | 76.5 | 69.6 | 10 | 0 | 2 | 0 |

Group 5: Bevacizumab + Docetaxel Dose: 10 + 10

| | | | | | | | |
|---|---|---|---|---|---|---|---|
| 1 | 5.4 | 79.2 | 75.2 | yes | no | no | TD |
| 2 | 6.6 | 105.9 | 102.1 | yes | no | no | TD |
| 3 | 23.3 | >160 | >158 | yes | no | no | TD |
| 4 | 3.2 | >160 | >59 | yes | no | no | TD |
| 5 | 10.8 | 96.3 | 92.2 | yes | no | no | TD |
| 6 | 29.0 | 65.6 | 62.0 | no | no | no | TD |
| 7 | 29.0 | >160 | >158 | no | no | no | TD |
| 8 | 31.1 | 87.1 | 69.3 | no | no | no | TD |
| 9 | 11.6 | 96.7 | 87.5 | yes | no | no | TD |
| 10 | NA | NA | NA | yes | no | no | NSD |
| Mean | 16.7 | NA | 95.9 | Total | Total | Total | Total |
| SD | 11.3 | NA | 37.7 | CR | PR | TFS | Excluded |
| Median | 11.6 | 96.7 | 87.5 | 7 | 0 | 0 | 0 |

Group 6: VGX-100 + Docetaxel Dose: 40 + 10

| | | | | | | | |
|---|---|---|---|---|---|---|---|
| 1 | 10.0 | NA | NA | yes | no | no | NSD |
| 2 | 11.9 | >160 | >156 | yes | no | no | TD |
| 3 | 10.5 | 100.5 | 94.5 | yes | no | no | TD |
| 4 | 10.2 | >160 | >162 | yes | no | no | TD |
| 5 | 6.0 | 87.4 | 82.7 | yes | no | no | TD |
| 6 | 17.1 | 99.6 | 89.8 | yes | no | no | TD |
| 7 | NA | >160 | >160 | yes | no | yes | TFS |
| 8 | 3.4 | NA | NA | yes | no | no | NSD |
| 9 | 11.9 | 86.3 | 79.5 | yes | no | no | TD |
| 10 | NA | >160 | >160 | yes | no | yes | TFS |
| Mean | 10.1 | NA | 123.0 | Total | Total | Total | Total |
| SD | 4.1 | NA | 39.2 | CR | PR | TFS | Excluded |
| Median | 10.4 | >130 | 125.2 | 10 | 0 | 2 | 0 |

Group 7: VGX-100 + Bevacizumab Dose: 40 + 10

| | | | | | | | |
|---|---|---|---|---|---|---|---|
| 1 | 6.0 | 24.5 | 21.1 | no | no | no | TD |
| 2 | 14.8 | 43.8 | 33.0 | no | no | no | TD |
| 3 | 7.0 | 21.8 | 19.1 | no | no | no | TD |
| 4 | 5.2 | 20.5 | 17.5 | no | no | no | TD |
| 5 | 15.0 | 32.0 | 26.3 | no | no | no | TD |
| 6 | 6.2 | 19.7 | 16.1 | no | no | no | TD |
| 7 | 40.3 | 77.0 | 53.9 | no | no | no | TD |
| 8 | 5.9 | 21.5 | 19.3 | no | no | no | TD |
| 9 | 14.7 | 32.0 | 21.2 | no | no | no | TD |
| 10 | 14.9 | 40.6 | 29.7 | no | no | no | TD |
| Mean | 13.0 | 33.3 | 25.7 | Total | Total | Total | Total |
| SD | 10.6 | 17.6 | 11.3 | CR | PR | TFS | Excluded |
| Median | 10.8 | 28.2 | 21.1 | 0 | 0 | 0 | 0 |

Group 8: VGX-100 + Bevacizumab + Docetaxel Dose: 40 + 10 + 10

| | | | | | | | |
|---|---|---|---|---|---|---|---|
| 1 | NA | NA | NA | yes | no | no | NSD |
| 2 | NA | >160 | >160 | yes | no | yes | TFS |
| 3 | 26.3 | >160 | >169 | yes | no | no | TD |
| 4 | 17.6 | >160 | >176 | yes | no | no | TD |
| 5 | NA | >160 | >160 | yes | no | yes | TFS |
| 6 | NA | >160 | >160 | yes | no | yes | TFS |
| 7 | 16.6 | 121.0 | 111.5 | yes | no | no | TD |
| 8 | NA | >160 | >160 | yes | no | yes | TFS |
| 9 | 22.8 | 142.9 | 124.7 | yes | no | no | TD |
| 10 | 12.4 | 136.8 | 132.1 | yes | no | no | TD |
| Mean | NA | NA | 150.4 | Total | Total | Total | Total |
| SD | NA | NA | 22.0 | CR | PR | TFS | Excluded |
| Median | NA | >160 | 160.0 | 10 | 0 | 4 | 0 |

LYVE-1 and CD31 immunohistochemistry staining of tumors harvested at various time-points throughout study will be performed.

Assessment of Side Effects

All mice were observed for clinical signs daily. The mice were weighed on each day of treatment and at least twice weekly thereafter. Individual body weights were recorded twice weekly.

Upon death or euthanasia, all mice were necropsied to provide a general assessment of potential cause of death and perhaps target organs for toxicity. The presence or absence of metastases was also noted. Individual and group toxicity findings have been summarized in Tables 1 and 2.

Results

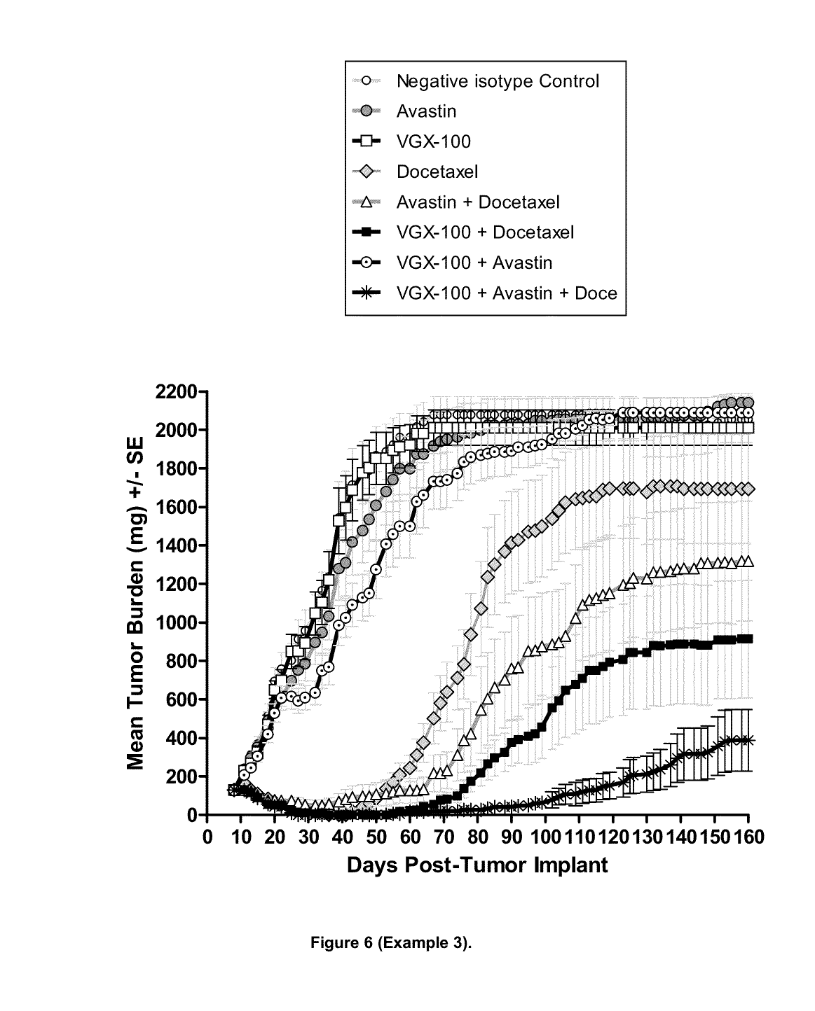

The results for Example 3 are provided in Tables 1 and 2 and FIGS. 6 to 9.

Tumor Growth/General Observations/Controls

A tumor burden of 750 mg was chosen for evaluation of efficacy by tumor growth delay. The Median Control Tumor reached evaluation size on Day 20, and the tumor volume doubling time for the Control Group was 6.1 days (Table 2). Control animals experienced an 11% weight loss by Day 25 with slow recovery thereafter. There were no spontaneous tumor regressions in the Control Group. All three thioglycolate cultures of tumor cells used for implantation of this study were negative for gross bacterial contamination. Based on historical data for this model, the biology of the Control Group was judged to be normal based on a historical tumor volume doubling time of approximately 7 days.

Efficacy

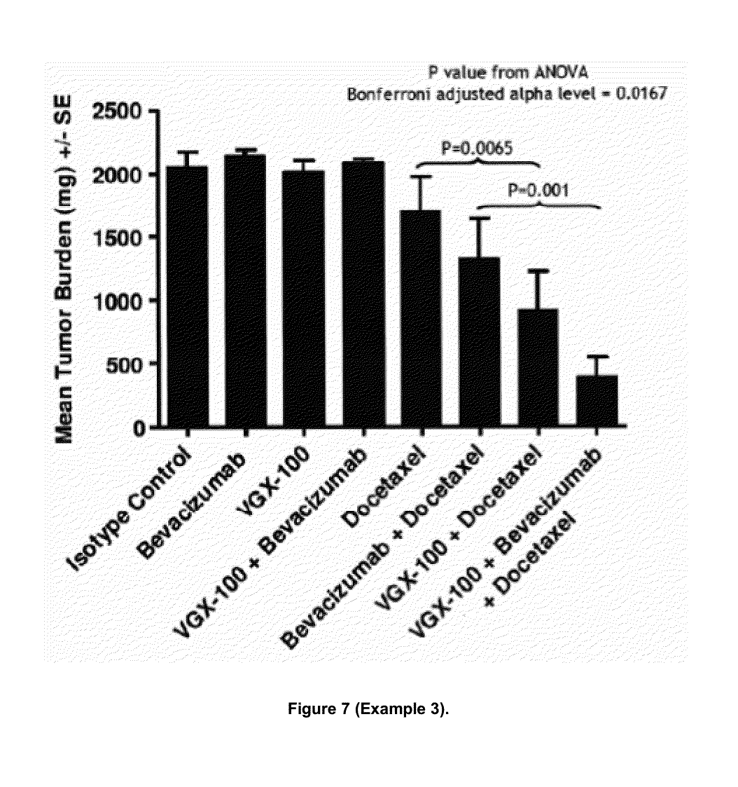

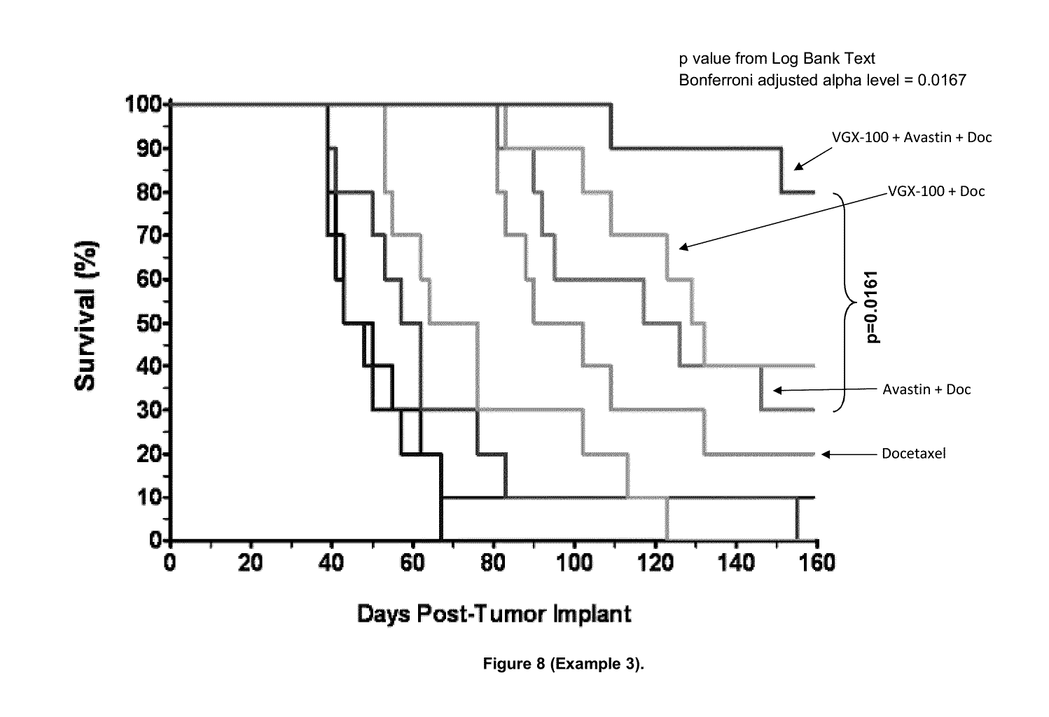

Primary measures of efficacy were tumor growth delay, Day 22 (the day on which the median control tumor burden reached 1 g), partial or complete tumor regressions, and tumor free survivors. Compiled efficacy data and graphics are contained in Tables 1 and 2, FIGS. 6 and 7. The survival rate of the mice is shown in FIG. 8.

Single Agents

Treatment with bevacizumab at 10 mg/kg, (Q3Dx2; 3 days off)×22, produced an insignificant (P>0.05) tumor growth delay of 5.6 days and a Day 22 T/C value of 70%. No tumor regressions were observed.

Treatment with VGX-100 at 40 mg/kg, (Q3Dx2; 3 days off)×9, was ineffective, producing an insignificant tumor growth delay of 2.2 days and a Day 22 T/C value of 87%. Nor tumor regressions were observed.

Treatment with docetaxel at 10 mg/kg, Q7Dx3, was highly active, producing a significant (P<0.05) tumor growth delay of 56.5 days, a positive net tumor cell kill value of 1.13 logs, a Day 22 T/C value of 8%, and an incidence of complete tumor regressions of 100%. Twenty percent of the responders remained tumor free at termination of the study on Day 160. The tumor growth delay produced by this regimen was statistically significant. The anti-cancer activity of this regimen was also statistically superior to that of bevacizumab and VGX-100.

Combination Regimens

Combination therapy with bevacizumab+docetaxel, both administered at 10 mg/kg, was highly active, producing a significant (P<0.05) tumor growth delay of 76.7 days, a Day 22 T/C value of 8%, and an incidence of complete tumor regressions of 70%. There were no tumor free survivors. The anti-cancer activity of this regimen was not statistically superior to that of treatment with docetaxel as a single agent based on analysis of tumor growth delay. The net tumor cell kill produced by this treatment regimen (−2.20 logs) was much lower than that produced by treatment with docetaxel as a single agent.

Combination therapy with VGX-100 at 40 mg/kg+docetaxel at 10 mg/kg, was highly active, producing a significant (P<0.05) tumor growth delay of 110.3 days, a Day 22 T/C value of 8%, and an incidence of complete tumor regressions of 100%. Twenty percent of the responders remained tumor free at termination of the study on Day 160. The anticancer activity of this regimen was statistically significant to that of treatment with docetaxel as a single agent based on analysis of the area under the curve (i.e. tumor burden over time). The net tumor cell kill produced by this treatment regimen (−2.06 logs) was much lower than that produced by treatment with docetaxel as a single agent.

Combination therapy with VGX-100 at 40 mg/kg+bevacizumab at 10 mg/k, produced an insignificant (P>0.05) tumor growth delay of 8.2 days, and a Day 22 T/C value of 76%. There were no tumor regressions. The anti-cancer activity of this regimen was not statistically superior to that of treatment with either bevacizumab or VGX-100 as a single agent based on analysis of tumor growth delay.

Combination therapy with VGX-100 at 40 mg/kg+bevacizumab at 10 mg/kg+docetaxel at 10 mg/kg was highly active, producing a significant (P<0.05) tumor growth delay of >140 days, a Day 22 T/C value of 7%, and an incidence of complete tumor regressions of 100%. Forty percent of the mice remained tumor free at termination of the study on Day 160. The tumor growth delay produced by this regimen was significantly longer than that of the binary combination of bevacizumab+docetaxel.

Discussion

FIG. 6 shows that VGX-100 significantly enhanced the anti-tumor efficacy of chemotherapy and bevacizumab in this prostate cancer model. Docetaxel was highly active against this model, as a single agent, producing long tumor growth delays, tumor regressions and tumor free survivors. Bevacizumab and VGX-100 were both active, however, adding VGX-100 to docetaxel resulted in a statistically significant improvement in tumor growth inhibition. Furthermore, adding VGX-100 to a combination of bevacizumab+docetaxel achieved a significant improvement in tumor growth inhibition compared to bevacizumab+docetaxel.

FIG. 7 summarises the tumor growth inhibition in the PC-3 model. At the conclusion of the experiment (day 160), tumors in the animals treated with the triple combination of VGX-100+bevacizumab+docetaxel were 16.5% the size of tumors in the control animals (see Table 3).

TABLE 3

| Treatment Group | % T/C^{a} |
|---|---|
| hIgG1 isotype control (40 mg/kg) | — |
| Bevacizumab (10 mg/kg) | 104.2% |
| VGX-100 (40 mg/kg) | 97.9% |
| VGX-100 (40 mg/kg) + bevacizumab (10 mg/kg) | 101.7% |
| Docetaxel (10 mg/kg) | 82.4% |
| Bevacizumab (10 mg/kg) + docetaxel (10 mg/kg) | 64.2% |
| VGX-100 (40 mg/kg) + docetaxel (10 mg/kg) | 44.5% |
| VGX-100 (40 mg/kg) + bevacizumab (10 mg/kg) + | 16.6% |
| docetaxel (10 mg/kg) | |

^{a}% T/C: tumor burden in treatment group compared to control treatment group at day 160

Tumor growth inhibition by the triple combination was superior to bevacizumab+docetaxel with a p-value of 0.001, which exceeds the standard for statistical significance of 0.0167. VGX-100+docetaxel was superior to docetaxel alone with a p-value of 0.0065, which also exceeds the threshold for statistical significance. p-values were calculated using ANOVA of the area under the curve measurements. The Bonferroni-adjusted alpha level of 0.0167 was determined as the threshold for statistical significance.

FIG. 8 summarises the survival rates of the mice in the different treatment groups. Addition of VGX-100 to docetaxel+bevacizumab improves survival in this prostate cancer model. The figure on the left represents the percentage of mice in each treatment group in the study that survived as a function of time. Survival of animals in the group treated with the triple combination of VGX-100+docetaxel+bevacizumab was the highest (80%), and exceeded that in the group treated only with docetaxel+bevacizumab by a statistically significant margin (p-value 0.0161). Table 4 summarizes average survival duration for each treatment group. 40% of animals treated with the triple combination had no detectable tumors at the conclusion of the study (Tumor Free Survivors, TFS). This compares to none of the animals treated with bevacizumab+docetaxel and 20% of the animals treated with docetaxel alone. Animals with tumors larger than 2 g were euthanized.

TABLE 4

| | Average | | | |
|---|---|---|---|---|
| | Survival | | | |
| Treatment Group | (days) | % CR^{a} | % PR^{b} | % TFS^{c} |
| hIgG1 isotype control | 59.9 | 0 | 0 | 0 |
| (40 mg/kg) | | | | |
| Docetaxel (10 mg/kg) | 108.6 | 100 | 0 | 20 |
| Bevacizumab (10 mg/kg) + | 122.7 | 70 | 0 | 0 |
| docetaxel (10 mg/kg) | | | | |
| VGX-100 (40 mg/kg) + | 131.8 | 100 | 0 | 20 |
| docetaxel (10 mg/kg) | | | | |
| VGX-100 (40 mg/kg) + | 154 | 100 | 0 | 40 |
| bevacizumab (10 mg/kg) + | | | | |
| docetaxel (10 mg/kg) | | | | |

^{a}% CR: Complete regression: Tumor burden reduced to an immeasurable volume at any point after the first treatment;
^{b}% PR: Partial regression: Tumor burden reduced to less than half of the tumor burden at the first treatment;
^{c}% TFS: Tumor free survival: Tumor burden is immeasurable at termination of the experiment (day 160)

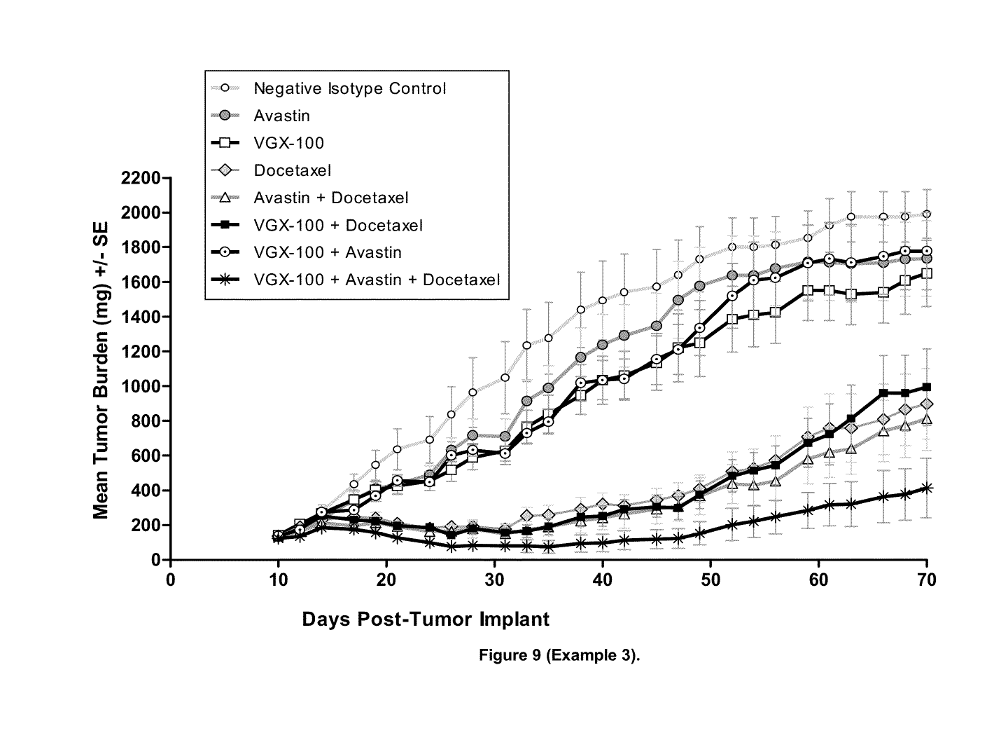

The results of a second study according to Example 3 are provided in FIG. 9.

### Example 4

### Combination Therapy in a Prostate Cancer (PC-3) Orthotopic Model

Experimental Method

Male SCID mice (3-4 weeks old) were obtained from ARC, Perth. Mice were injected with an analgesic (carprofen) and anaesthetized with ketamine/xylazine followed by isoflurane maintenance. The prostate was exposed surgically and injected with 10⁵ cells in 10 μl.

General mouse health was monitored using twice weekly weighing and visual inspection.

Tumor growth and dissemination was monitored by palpation, and longitudinal in vivo imaging (combined x-ray and fluorescence imaging performed at least once per week).

The experiment was continued for 8 weeks, unless deterioration in mouse health or excessive tumor burden was recorded. At this time mice were anaesthetized, blood collected by cardiac puncture, tumors weighed and measured, final images generated and organs harvested for histological analysis. Metastases were also studied.

### Experimental Design

N=12 per group as follows. Dosing for VGX-100 was twice to three times per week.

Dosing for docetaxel and anti-angiogenic therapy was determined. Dosing was initiated 1 day after tumor inoculation.

A. PC-3 Tumor Xenograft: Single Agent Dose Response Experiment.

Groups A-H were included in the single therapy PC-3 xenograft study (Table 5).

TABLE 5

| Group | Treatment | Dose | # Mice |
|---|---|---|---|
| A | VEGF-C antibody | 5 mg/kg | 12 |
| B | VEGF-C antibody | 10 mg/kg | 12 |
| C | VEGF-C antibody | 20 mg/kg | 12 |
| D | VEGF-C antibody | 40 mg/kg | 12 |
| E | Docetaxol low dose | TBD | 12 |
| F | Docetaxol medium dose | TBD | 12 |
| G | Docetaxol high dose | TBD | 12 |
| H | hIgG1 isotype control | 40 mg/kg | 12 |
| I | Bucalutamide submax dose | TBD | 12 |
| J | Bucalutamide max dose | TBD | 12 |
| K | GNRH Agonist submax dose | TBD | 12 |
| L | GNRH Agonist max dose | TBD | 12 |

Submaximal and maximal dose of VGX-100, docetaxol, bucalutamide and GNRH as single-agents were determined.

B. PC-3 Tumor Xenograft: Combination with Chemotherapy in Dose Response Experiment.

Groups A-E were included in the combination therapy PC-3 xenograft study (Table 6).

TABLE 6

| Group | Treatment | Dose | # Mice |
|---|---|---|---|
| A | VEGF-C antibody submax dose | ~5-10 mg/kg | 12 |
| | | (TBD) | |
| B | VEGF-C antibody max dose | ~40 mg/kg | 12 |
| | | (TBD) | |
| C | Docetaxol submax dose | TBD | 12 |
| D | VEGF-C antibody + Docetaxol | TBD | 12 |
| | submax doses | | |
| E | hIgG1matched high dose | TBD | 12 |
| F | Bucalutamide submax dose | TBD | 12 |
| G | GNRH Agonist submax dose | TBD | 12 |
| H | VEGF-C antibody + Bucalutamide | TBD | 12 |
| | submax doses | | |
| I | VEGF-C antibody + GNRH Agonist | TBD | 12 |
| | submax doses | | |

C. PC-3 Tumor Xenograft: Combination with a-Angiogenic Tx (e.g. Bevacizumab) Dose Response Experiment.

Groups A-F will be included in the combination therapy PC-3 xenograft study (Table 7).

TABLE 7

| Group | Treatment | Dose | # Mice |
|---|---|---|---|
| A | VEGF-C antibody submax dose | ~5-10 mg/kg | 12 |
| | | (TBD) | |
| B | VEGF-C antibody max dose | ~40 mg/kg | 12 |
| | | (TBD) | |
| C | a-Angio Tx submax dose | TBD | 12 |
| | | (Avastin = ~5 | |
| | | mg/kg) | |
| D | a-Angio Tx mas dose | TBD | 12 |
| | | (Avastin = ~20 | |
| | | mg/kg) | |
| E | VEGF-C antibody + a-Angio | TBD | 12 |
| | Tx submax doses | | |
| F | hIgG1 matched high dose | TBD | 12 |

Monitoring Tumor Growth and Metastases

Tumor dimensions were recorded no less than weekly by palpation and longitudinal in vivo imaging employing combined x-ray and fluorescence methods.

The tumor was cut through the middle in a consistent and reproducible manner, so as to obtain sections that were representative of the entire tumor cross-section. Samples of primary and metastatic tumors were taken and analyzed as follows:
Frozen sections: CD31 staining.
Paraffin blocks: Tumor sample was fixed in formalin and embedded in paraffin. Sections were stained using H&E, a-LYVE-1 antibody (to detect lymphatic vessels) and a-CD34 (to detect blood vessels).
IHC/IF analysis: In addition to a-LYVE-1, a-CD34 (or a-CD31) staining, VEGF, VEGF-C, VEGF-D, VEGFR-1, VEGFR-2 and VEGFR-3 protein levels and localization were determined by IHC/IF methods. Both FFPE and frozen sections were used in this analysis (depending on the characteristics of the available antibodies.
Serum Samples: Serum was collected at the termination of the experiment by cardiac puncture. PSA levels were not measured as PC-3 cells do not produce PSA. The remaining serum was frozen for future analysis.

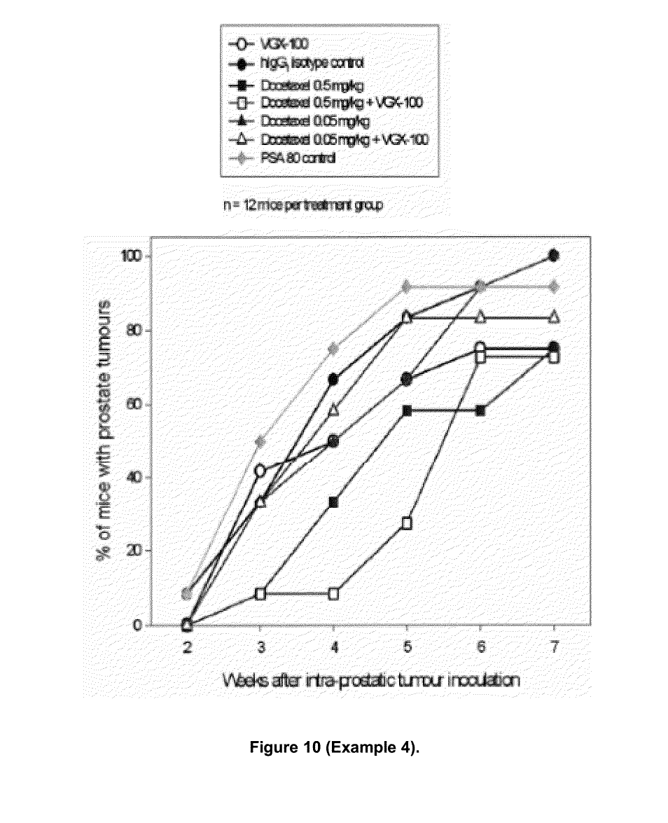

The results of Example 4 are provided in FIG. 10.

### Example 5

### Combination Therapy in a Prostate Cancer (LNCaP) Orthotopic Model

The same protocol as Example 4 will be followed except that LNCaP prostate cancer cells will be substituted for PC-3 prostate cancer cells. In the Single Agent Dose Response Experiment (A), groups A-L will be included in the LNCaP study, in line with Table 3. In the Combination with Chemotherapy Dose Response Experiment (B), groups A-I will be included in the LNCaP study, in line with Table 6.

PSA levels will be measured in the LNCaP study of Example 5.

### Example 6

### Single Agent Therapy in a Glioblastoma (U87MG) Xenograft Model

To initiate the study, 5×10⁶ of U-87 cells suspended in 100 μl of a mixture of medium/Matrigel (1:1) were subcutaneously implanted to the nude mice in the right flank region. Cancer cells were planted into 80 mice. Animals were monitored for tumor growth daily after cell implantation. When tumor volumes reached 80-100 mm³, mice were randomized into 5 groups of 10 mice each using only mice having tumor volumes closest to the mean value. Mice with tumor volumes too big or too small were excluded from the study. Tumor volumes were measured using the formula V=L×W×H×π/6, where L and W represented the longer and shorter diameters of the tumor and H represented the height of the tumor. The treatment with drugs was initiated on the day after randomization. Vehicle control, test antibody and bevacizumab were administered by intraperitoneal injection in volumes ranging between 64 and 108 μl per dose. Bevacizumab (Genentech) was supplied as drug in aqueous solution at a concentration of 25 mg/ml and diluted 10-fold with PBS. The treatment was carried out twice weekly for 27 days. Throughout the entire study, tumor volumes were measured twice weekly and body weights once weekly. Animals were also observed for possible toxic effects from the drug treatment. Unscheduled sacrifices were performed if tumor volumes reached >1,500 mm³, animals lost >25% of their original body weights, tumor ulceration appeared, or mice became moribund. At the end of the experiment, tumors were removed, embedded in Optimal Cutting Temperature (OCT), and stored at −80° C. for IHC analysis. Gross examination was conducted on liver, lung, spleen, kidneys, testes, and prostate. If no abnormal observation was detected on these organs, the tissues were discarded.

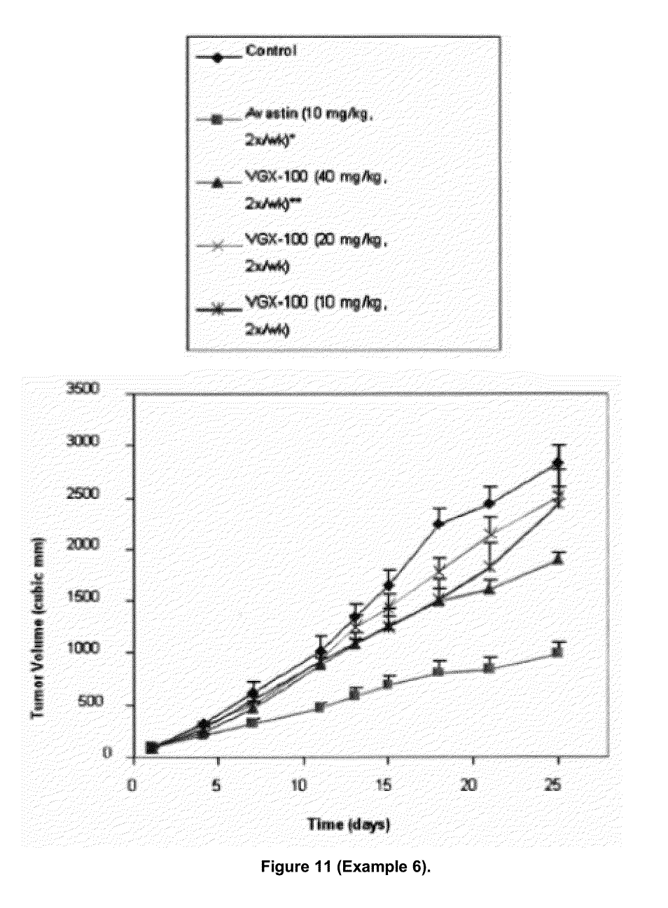

The VEGF-C antibody VGX-100 (40 mg/kg) significantly inhibited tumor growth compared to vehicle control in the single-agent U87MG xenograft study (FIG. 11).

### Example 7

### Combination Therapy in a Glioblastoma (U87MG) Xenocraft Model

This experiment was conducted broadly in accordance with Example 3, except that U87MG glioblastoma cells were substituted for PC-3 prostate cancer cells. Cells from a human glioblastoma tumor line (U87MG) were implanted subcutaneously into mice and grown until the tumors reached an average size of approximately 150 mg. Mice were treated twice weekly with either VGX-100 (40 mg/kg), bevacizumab (10 mg/kg), a combination of the two, or a negative control antibody (Isotype Control). Tumor size was measured 2-3 times weekly with calipers. Vertical bars indicate the standard error of the mean for tumor weight for each time point in each treatment group. There were 10 animals per treatment group.

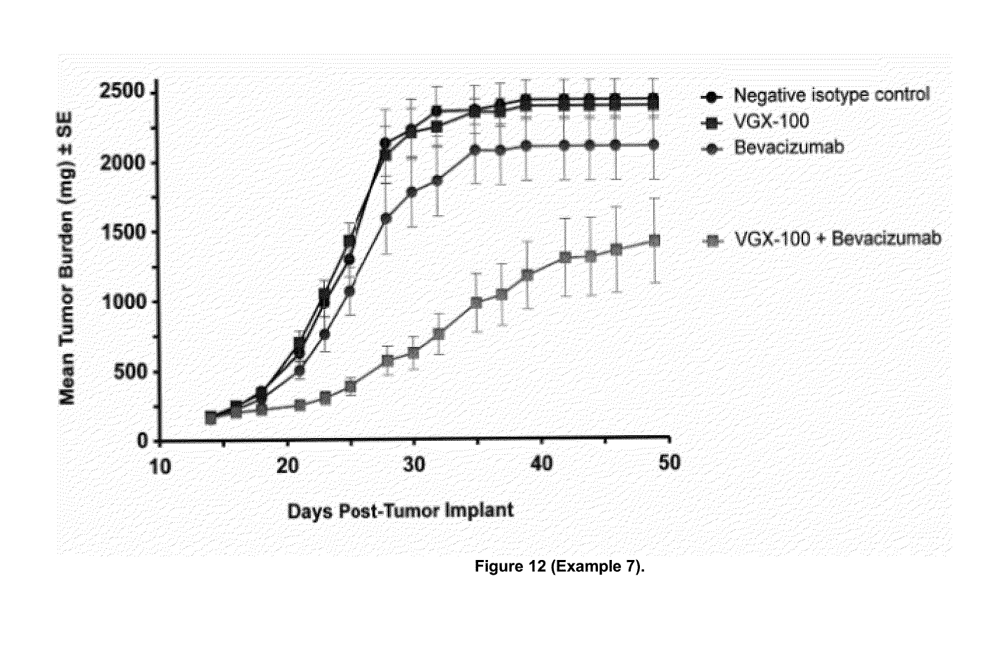

Administered alone, bevacizumab had only a minor effect in slowing the growth of U87MG brain cancer tumors, and VGX-100 had no significant effect. Used in combination however, VGX-100 plus bevacizumab achieved a 42% reduction in tumor growth at day 49 (see FIG. 12).

### Example 8

### Combination Therapy in a Pancreatic Cancer (KP4) Xenocraft Model

To initiate the study, 5×10⁶ of KP4 cells suspended in 100 μl of a mixture of medium/Matrigel (1:1) were subcutaneously implanted to the nude mice in the right flank region. Cancer cells were implanted into 90 mice. Animals were monitored for tumor growth daily after cell implantation. When tumor volumes reached 80-100 mm³, mice were randomized into 5 groups of 10 mice each using only mice having tumor volumes closest to the mean value. Mice with tumor volumes too big or too small were excluded from the study. Tumor volumes were measured using the formula V=L×W×H×π/6, where L and W represented the longer and shorter diameters of the tumor and H represented the height of the tumor. The treatment with drugs was initiated on the day after randomization. Vehicle control, test antibody and bevacizumab were administered by intraperitoneal injection in volumes ranging between 68 and 122 μl per dose. Bevacizumab was supplied as drug in aqueous solution at a concentration of 25 mg/ml and diluted 10-fold in PBS. All treatments were administered intraperitoneally twice weekly for 30 days. Mice were treated with either VGX-100 (at 20 or 40 mg/kg), bevacizumab (10 mg/kg), a combination of the two (VGX-100 at 40 mg/kg+bevacizumab at 10 mg/kg), or a negative isotype control antibody.

Throughout the entire study, tumor volumes were measured twice weekly and body weights once weekly. Animals were also observed for possible toxic effects from the drug treatment. Unscheduled sacrifices were performed if tumor volumes reached >2,000 mm³, animals lost >25% of their original body weights, tumor ulceration appeared, or mice became moribund. At the end of the experiment, tumors were removed, embedded in Optimal Cutting Temperature (OCT), and stored at −80° C. for IHC analysis. Gross examination was conducted on liver, lung, spleen, kidneys, testes, and prostate. If no abnormal observation was detected on these organs, the tissues were discarded.

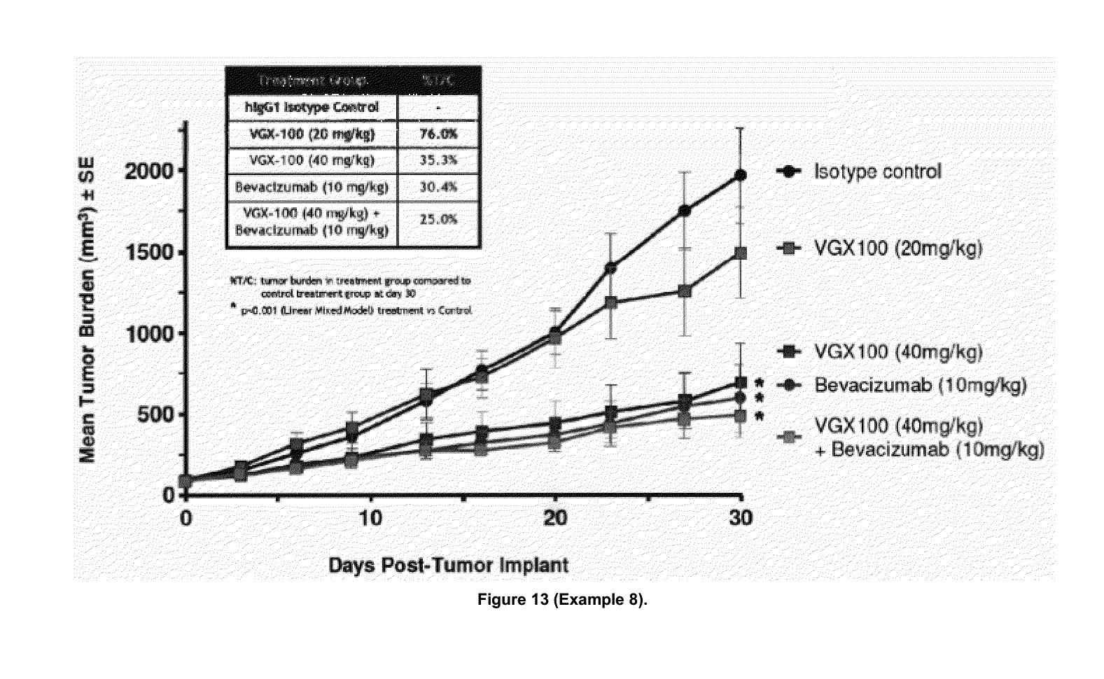

FIG. 13 shows the mean tumor burden in each treatment group over time. Pancreatic tumors in mice treated with 40 mg/kg VGX-100 were on average 35.3% the size of tumors in control mice. This is similar to the size (30.4%) of tumors treated with bevacizumab. An asterisk (*) indicates that there is a statistically significant difference in the average size of tumors in the treatment group compared to the control (analysis by ANOVA). Tumor size was measured 2-3 times weekly with calipers. Size of tumors relative to controls is calculated after 30 days of treatment. Vertical bars indicate the standard error of the mean for tumor weight for each time point in each treatment group.

TABLE 8

| Treatment Group | Mean Tumor Vol. at D 30 (mm3) | % T/C |
|---|---|---|
| hIgG1 Isotype Control | 1964.31 | — |
| (40 mg/kg) | | |
| Avastin (10 mg/kg) | 596.7 | 30.4% |
| VGX-100 (40 mg/kg) + | 490.76 | 25.0% |
| Avastin (10 mg/kg) | | |
| VGX-100 (20 mg/kg) | 1492.77 | 76.0% |
| VGX-100 (40 mg/kg) | 693.24 | 35.3% |
| VGX-100 (60 mg/kg) | 958.93 | 48.8% |

LYVE-1 and CD31 immunohistochemical staining and quantitation on lymph node tissue was performed. Immunohistochemical staining & quantitation of blood and lymphatic vessels on formalin fixed frozen KP4 tumors will be conducted.

### Example 10

### Colorectal Carcinoma (HCT-116) Combination Therapy Model

This experiment was conducted broadly in accordance with Example 3, except that HCT-116 colorectal carcinoma cells were substituted for PC-3 prostate cancer cells and fluorouracil was substituted for docetaxel as the chemotherapeutic agent.

Mice were divided into the following treatment groups: Isotype control; bevacizumab alone; VGX-100 alone; 5-FU alone; bevacizumab+5-FU; VGX-100+5-FU; VGX-100+bevacizumab; VGX-100+bevacizumab+5-FU. Where used, 5-FU was administered twice weekly (at 50 mg/kg) intravenously for the duration of the experiment, VGX-100 was administered twice weekly (at 40 mg/kg) intraperitoneally for the duration of the experiment and bevacizumab was administered twice weekly (at 10 mg/kg) intraperitoneally for the duration of the experiment.

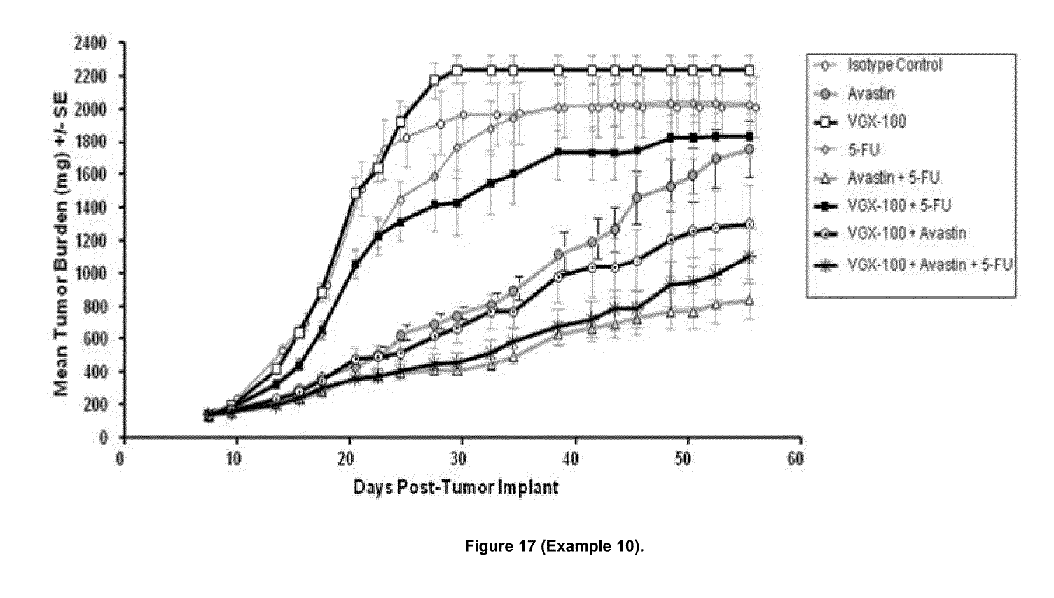

The results are presented in FIG. 17. A small additive effect of the VEGF-C antibody VGX-100 was observed in combination with Bevacizumab against tumors. Likewise, there was evidence of a small additive effect when VGX-100 is used in combination with fluorouracil chemotherapy.

### Example 11

### Luna Carcinoma (H292) Combination Therapy Tumor Model

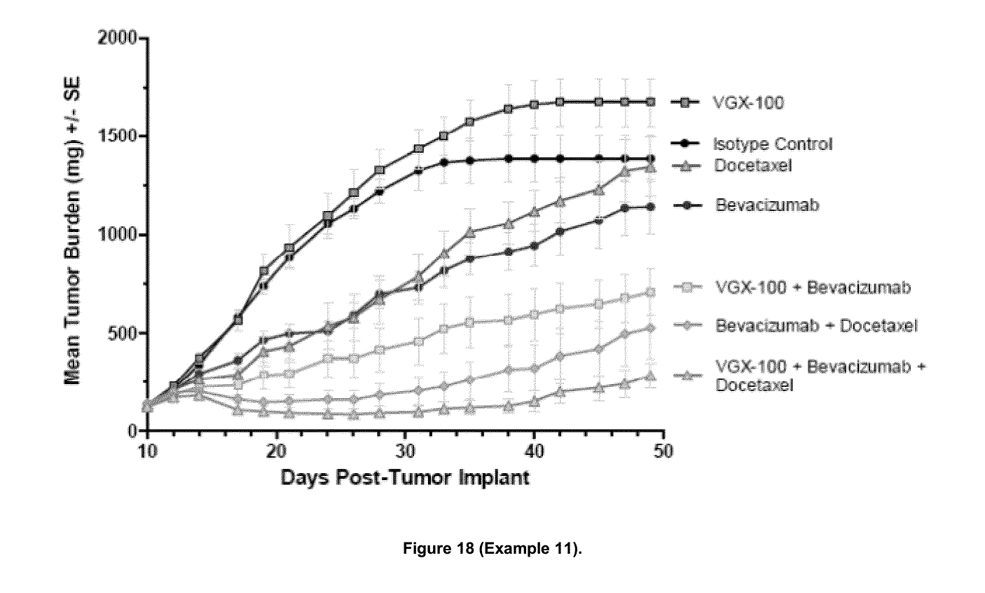

A similar protocol as that described in Example 3 was followed. H292 (5×10⁵) cells were implanted subcutaneously in nu/nu mice high in the right axilla. Mice were triaged into treatment groups (n=10/group) when the mean tumor burden was 75-175 mg. Tumor burden was estimated from caliper measurements by the formula: Tumor burden (mg)=(L×W2)/2, where L and W are the respective orthogonal tumor length and width measurements (mm). Antibodies were administered 2×/week via intraperitoneal injection (Isotype control and VGX-100, 40 mg/kg; bevacizumab, 10 mg/kg). Docetaxel (10 mg/kg) was administered intravenously weekly for three weeks. The results are presented in FIG. 18.

### Example 12

### Ovarian Carcinoma (OVCAR-8) Combination Therapy Model

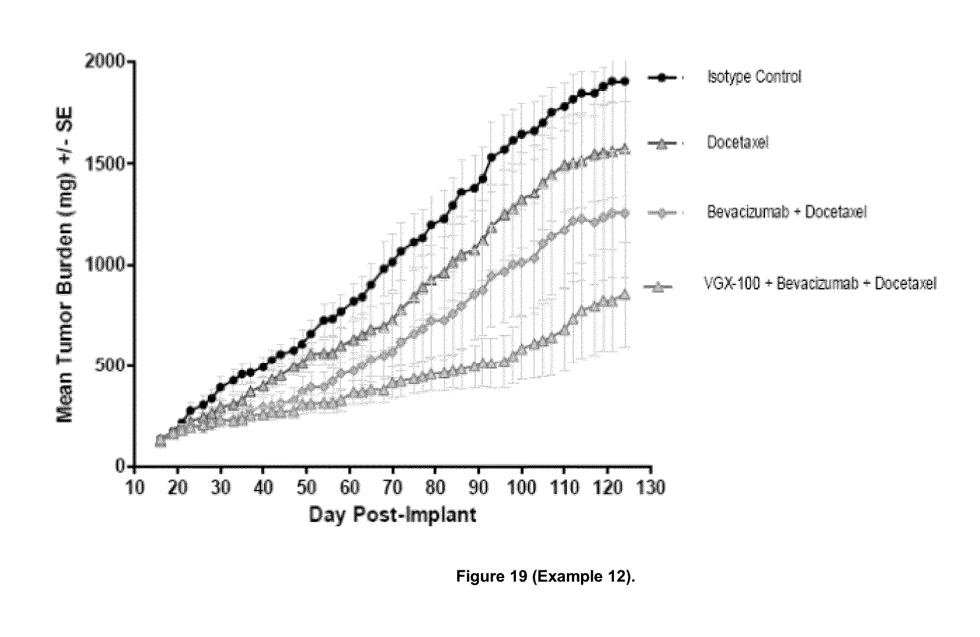

A similar protocol as that described in Example 3 was followed. OVCAR-8 (1×10⁷) cells were implanted subcutaneously in nu/nu mice high in the right axilla. Mice were triaged into treatment groups (n=10/group) when the mean tumor burden was 75-175 mg. Tumor burden was estimated from caliper measurements by the formula: Tumor burden (mg)=(L×W2)/2, where L and W are the respective orthogonal tumor length and width measurements (mm). Antibodies were administered 2×/week via intraperitoneal injection (Isotype control and VGX-100, 40 mg/kg; bevacizumab, 10 mg/kg). Docetaxel (10 mg/kg) was administered intravenously weekly for three weeks. The results are presented in FIG. 19.

### Example 13

### Single Agent Therapy in a Prostate Cancer (PC-3) Orthotopic Model

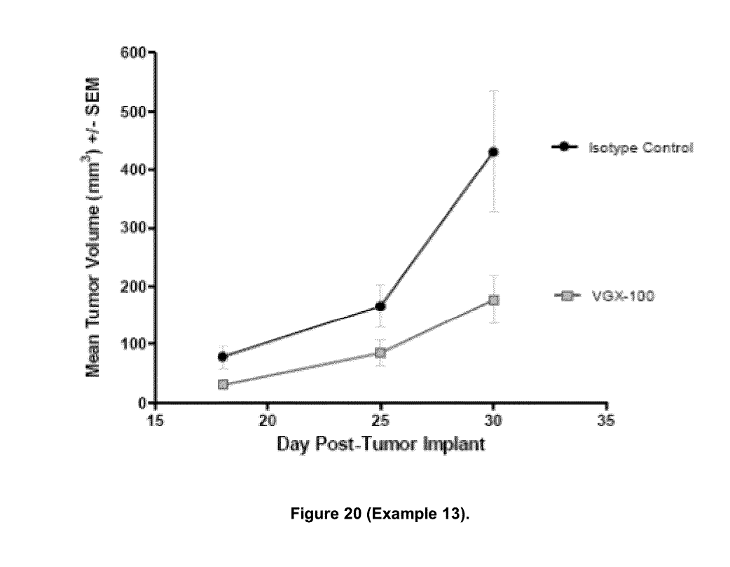

PC-3-GFP human prostate cancer orthotopic MetaMouse® model was conducted by AntiCancer Inc. PC-3-GFP tumor fragments were surgically implanted between the ventral lobes of the prostate and closed by suture. Treatment was started three days after surgery (60 mg/kg, 3×/week, IP). Whole body imaging of GFP-expressing tumors was performed once a week in live animals after GFP-visible tumors were established. Primary tumor sizes were estimated once a week by caliper measurement and tumor volume (mm³) calculated by the formula (L×W2)/2. The results are presented in FIG. 20. Details of the study are as follows:

### Experimental Animals:

Male NCr nu/nu mice, 5-6 weeks old, were used in the study. Original breeding pairs were purchased from Taconic, Germantown, N.Y. Test animals were bred and maintained in a HEPA filtered environment for the experiment. Cages, food and bedding were autoclaved. The animal diets were obtained from PMI Nutrition International Inc. (Brentwood, Mo.).

### Study Compounds and Drug Preparation:

Vehicle, VGX-100 and Isotype were kept at −20° C. until use. VGX-100 and Isotype were diluted with PBS to proper concentration (7.5 mg/ml and 7.5 mg/ml) before use.

### PC-3-GFP Human Prostate Cancer Orthotopic MetaMouse® Model:

Human prostate cancer cell line P-C-3 was purchased from ATCC.
a. GFP Expression Vector:
The pLEIN retroviral vector (CLONTECH) expressing enhanced GFP and the neomycin resistance gene on the same bicistronic message, which contains an internal ribosome entry site, was used to transduce tumor cells.
b. Packaging Cell Culture, Vector Production, Transfection, and Subcloning:
PT67, an NIH 3T3-derived packaging cell line expressing the 10 Al viral envelope, was purchased from CLONTECH. PT67 cells were cultured in DMEM (Irvine Scientific) supplemented with 10% heat-inactivated FBS (Gemini Biological Products, Calabasas, Calif.). For vector production, packaging cells (PT67), at 70% confluence, were incubated with a precipitated mixture of N-[1-(2,3-dioleoyloxy)propyl]-N,N,N-trimethylammonium methylsulfate reagent (Roche Molecular Biochemicals) and saturating amounts of pLEIN plasmid for 18 h. Fresh medium was replenished at this time. The cells were examined by fluorescence microscopy 48 h after transfection. For selection, the cells were cultured in the presence of 500-2000 μg/ml of G418 (Life Technologies, Grand Island, N.Y.) for 7 days.
c. Retroviral GFP Transduction of Tumor Cells:
For GFP gene transduction, 25% confluent PC-3 cells were incubated with a 1:1 precipitated mixture of retroviral supernatants of PT67 cells and RPMI 1640 (GIBCO) containing 10% FBS (Gemini Biological Products) for 72 h. Fresh medium was replenished at this time. Cells were harvested by trypsin-EDTA 72 h after transduction and subcultured at a ratio of 1:15 into selective medium, which contained 200 μg/ml of G418. The level of G418 was increased to 400 μg/ml stepwise. Clones stably expressing GFP were isolated with cloning cylinders (Bel-Art Products) with the use of trypsin-EDTA and were then amplified and transferred by conventional culture methods.
d. Subcutaneous Xenograft:
Tumor stocks were made by subcutaneously injecting PC-3-GFP cells at a concentration of 5×106 cells/100 μl into the flank of nude mice. The tumor tissues harvested from s.c. growth in nude mice were inspected and any grossly necrotic or suspected necrotic or non-GFP tumor tissues were removed. Tumor tissues were subsequently cut into small fragments of approximately 1 mm³.
e. Surgical Orthotopic Implantation (SOI).
The animals were anesthetized with the mixture of Ketaset, Xylazine and PromAce and the surgical area was sterilized using iodine and alcohol. An incision approximately 2 cm long was made in the lower abdomen of the nude mouse using a sterile scalpel and blade (#10). The prostate was exposed and the capsule of the prostate was carefully opened. Two fragments (1 mm³ each) of PC-3-GFP tissue were sutured between two ventral lobes of the prostate with sterile 8-0 surgical suture (nylon). The abdomen was closed in one layer with sterile 6-0 surgical sutures (silk). All procedures were carried out under a 5× dissecting microscope under HEPA filtered laminar flow hoods.

### Study Design:

Treatment in both groups was started three days after SOI. Table 9 shows the study design.

TABLE 9
Study Design

| Group | Agent | Dose | Schedule | Route | # |
|---|---|---|---|---|---|
| 1 | Isotype | 60 mg/kg | Three times per week | IP | 20 |
| | Antibody | | | | |
| | Control | | | | |
| 2 | VGX-100 | 60 mg/kg | Three times per week | IP | 20 |

### Fluorescence Optical Tumor Imaging (FOTI):

The FluorVivo imaging system (INDEC Biosystems, Santa Clura, Calif.) was used for whole body imaging. Whole body optical imaging of GFP-expressing tumors was performed once a week in live animals after GFP-visible tumors were established. At necropsy, open imaging was performed in the thoracic and abdomen for inspection of metastasis to the lymph nodes.

Tumor size and body weight measurement: Primary tumor sizes and body weights were measured once a week by using a calipers and an electronic scale, respectively. Primary tumor sizes were estimated once a week by measuring the perpendicular minor dimension (W) and major dimension (L). Approximate tumor volume (mm³) was calculated by the formula (W2×L)×1/2.

### Study Endpoint:

All the animals were sacrificed at day 30 after treatment initiation.

### Final Tumor Weight:

Primary tumor was excised and weighed at necropsy.

### Tissue Collection:

Primary prostate tumor and any organs with metastasis were harvested. Tumors and organs were cut symmetrically in order to have equal representation of the tumor in each half. One half of each tumor and each organ was fixed in 10% NBF. The other half of the tumor and each organ were snap frozen in liquid nitrogen.

### Statistical Analysis Used in the Study:

The Student's t-test with a equal to 0.05 was used to compare the mean tumor volume and tumor weight among the experimental groups.

### Results

Animals in this study were sacrificed on day 30 after treatment. Tumor volume and tumor weight were analyzed with Student's t-test to compare the vehicle control and treated Groups.

### 1. Effect on Tumor Volume:

On day 28 after treatment, the mean tumor volume of the treated Group was compared to that of the control Group. Tumor volumes showed a statistically significant reduction (p<0.05) in the treated Group compared to the isotype control Group (see Table 10).

TABLE 10
Mean tumor volume on day 28 after
treatment (caliper measurement)

| | | # of | Mean tumor | |
|---|---|---|---|---|
| | | animals | volume (mm³) | |
| Group | Agent | available | mean ± SE | P-value* |
| 1 | Isotype | 18 | 430.8 ± 464.55 | — |
| | antibody | | | |
| | control | | | |
| 2 | VGX-100 | 19 | 176.8 ± 183.2 | 0.019 |

*Student's t-test was used to compare between the treated group and vehicle control group. A difference is considered statistically significant with a P ≦ 0.05.

### 2. Effect on Tumor Weight:

The mean tumor weight of the treated group was compared to that of the control group. A statistically significant reduction was not observed in the treated group compared to the isotype control group on day 30 after treatment (see Table 11).

TABLE 11
Mean tumor weight on day 30 after treatment

| | | # of | Mean tumor | |
|---|---|---|---|---|
| | | animals | weight (g) | |
| Group | Agent | available | mean ± SE | P-value* |
| 1 | Isotype | 17 | 1.15 ± 0.75 | — |
| | antibody | | | |
| | control | | | |
| 2 | VGX-100 | 19 | 0.95 ± 0.45 | 0.11 |

*Student's t-test was used to compare between the treated group and vehicle control group. A difference is considered statistically significant with a P ≦ 0.05.

### 3. Effect on Metastasis:

All study animals were opened at sacrifice. Optical imaging of GFP-expressing metastases was performed. The metastatic incidence was analyzed by FOTI and the Fisher's exact test. A significant difference in metastatic incidence to lymph nodes (LNs) was found between the treated Group and the control Group (P=0.019) (see Table 12).

TABLE 12
Efficacy of Treatment on Metastasis Analyzed by FOTI

| | | | | # of | |
|---|---|---|---|---|---|
| | | # of | # of | animals | |
| | | animals | analyzed | with lymph | |
| Group | Agent | in group | animals | node (LN) mets | P-value^{a} |
| 1 | Isotype | 20 | 17 | 12 (71%) | — |
| | antibody | | | | |
| | control | | | | |
| 2 | VGX-100 | 20 | 19 | 6 (32%) | 0.019 |

^{a}P value from treated group compared to untreated control by Fisher exact test.

### 4. Estimation of Toxicity:

A stable body weight in antibody treatment groups without significant loss compared to vehicle controls indicated that antibody VGX-100 had no obvious toxicity at these experimental doses.

### CONCLUSION

Based on the results on day 28 after treatment, mean tumor volume showed a statistically significant reduction (a 59% reduction) in the treated Group [VGX-100 (60 mg/kg), IP] compared to the control Group [Isotype (60 mg/kg), IP)]. VGX-100 also reduced incidence of metastatis to local lymph nodes by 55% in the treated Group compared to the control group. VGX-100 and Isotype had no obvious toxicity at these experimental doses. No acute body weight loss was observed in the treated Group during the entire study.

1. A method of treating cancer in a human subject, comprising administering to the subject in combination therapeutically effective amounts of a VEGF-C antagonist and an anti-neoplastic composition.

2. The method of claim 1, wherein the VEGF-C antagonist is selected from a VEGF-C antibody, a VEGF-C variant, a VEGFR-3 antibody, a receptor specific for VEGF-C, a VEGFR-3 receptor, an aptamer capable of blocking VEGF-C or a receptor specific for VEGF-C, and a low molecular weight inhibitor of a VEGFR-3 tyrosine kinase.

3. The method of claim 2, wherein the VEGF-C antibody binds the same epitope as the monoclonal VEGF-C antibody 69D09 produced by hybridoma ATCC PTA-4095.

4. The method of claim 2 or claim 3, wherein the VEGF-C antibody is a human antibody.

5. The method of claim 2 or claim 3, wherein the VEGF-C antibody is a humanized antibody.

6. The method of claim 5, wherein the VEGF-C antibody is a humanized 69D09 antibody or fragment thereof.

7. The method of claim 2 or claim 3, wherein the VEGF-C antibody comprises a heavy chain provided as SEQ ID NO: 3 and/or a light chain provided as SEQ ID NO: 4.

8. The method of any one of claims 2 to 7, wherein the VEGF-C antibody is monoclonal.

9. The method of any one of claims 2 to 8, wherein the VEGF-C antibody is administered intravenously.

10. The method of any one of claims 1 to 9, comprising anti-angiogenic therapy or anti-lymphangiogenic therapy.

11. The method of claim 10, wherein the anti-angiogenic therapy comprises administering to the subject an anti-angiogenic agent.

12. The method of any one of claims 1 to 9, wherein the anti-neoplastic composition comprises an anti-angiogenic agent.

13. The method of claim 11 or claim 12, wherein the anti-angiogenic agent comprises an anti-angiogenic antibody.

14. The method of claim 13, wherein the anti-angiogenic antibody comprises a VEGF-A antibody.

15. The method of claim 14, wherein the VEGF-A antibody binds the same epitope as the monoclonal VEGF-A antibody A4.6.1 produced by hybridoma ATCC HB 10709.

16. The method of claim 14 or claim 15, wherein the VEGF-A antibody is a human antibody or a humanized antibody.

17. The method of claim 16, wherein the VEGF-A antibody is a humanized A4.6.1 antibody or fragment thereof.

18. The method of claim 17, wherein the VEGF-A antibody is Bevacizumab.

19. The method of any one of claims 12 to 18, wherein the anti-angiogenic antibody is monoclonal.

20. The method of any one of claims 12 to 19, wherein the anti-angiogenic antibody is administered intravenously.

21. The method of any one of claims 1 to 20, wherein the anti-neoplastic composition comprises a chemotherapeutic agent.

22. The method of claim 21, wherein the chemotherapeutic agent is selected from alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog.

23. The method of claim 21 or claim 22, wherein the chemotherapeutic agent is selected from the group consisting of docetaxel, 5-fluorouracil (5-FU), temozolomide (TMZ), gemcitabine, oxaliplatin, paclitaxel, carboplatin and irinotecan.

24. The method of any one of claims 1 to 23, wherein the anti-neoplastic composition comprises an anti-angiogenic agent and a chemotherapeutic agent.

25. The method of claim 24, wherein the anti-angiogenic agent is a VEGF-A antagonist.

26. The method of claim 24 or claim 25, wherein the chemotherapeutic agent is selected from docetaxel, 5-fluorouracil (5-FU), temozolomide (TMZ), gemcitabine, oxaliplatin, paclitaxel, carboplatin and irinotecan and the anti-angiogenic agent is bevacizumab.

27. The method of any one of claims 1 to 23, wherein the anti-neoplastic composition comprises at least two chemotherapeutic agents.

28. The method of any one of claims 1 to 23, wherein the anti-neoplastic composition comprises a VEGF-C antagonist.

29. The method of any one of claims 1 to 28, further comprising administering to the subject another antagonist of tumor growth.

30. The method of claim 29, wherein the another antagonist of tumor growth is an antagonist of EGFR, ErbB2 (HER2), ErbB3, ErbB4, TNF, VEGF-A or a VEGFR.

31. The method of any one of claims 1 to 30, further comprising administering to the subject a cytokine, a cytotoxic agent, a growth inhibitory agent, or a small molecule VEGFR antagonist.

32. The method of any one of claims 1 to 31, comprising a standard of care for the cancer to be treated.

33. The method claim 32, wherein the standard of care comprises a standard chemotherapeutic agent for the cancer to be treated.

34. The method of claim 33, wherein the standard chemotherapeutic agent is selected from docetaxel, 5-fluorouracil (5-FU), temozolomide (TMZ), gemcitabine, oxaliplatin, paclitaxel, carboplatin and irinotecan.

35. The method of claim 34, wherein the standard chemotherapeutic agent is 5-FU and the method further comprises administering to the subject leucovorin.

36. The method of any one of claims 1 to 35, wherein the cancer is primary, or is stage I or stage II.

37. The method of any one of claims 1 to 35, wherein the cancer is metastatic or is stage III or stage IV.

38. The method of any one of claims 1 to 37, wherein the cancer is not respectable.

39. The method of any one of claims 1 to 38, wherein the cancer is resistant.

40. The method of claim 39, wherein the cancer is resistant to a VEGF-A antagonist.

41. The method of claim 39 or claim 40, wherein the cancer is resistant to bevacizumab.

42. The method of any one of claims 1 to 41, wherein the cancer is recurrent.

43. The method of claim 42, wherein the cancer is locally recurrent.

44. The method of any one of claims 1 to 43, wherein the cancer comprises a solid tumor.

45. The method of claim 44, wherein the solid tumor is vascularized.

46. The method of claim 44 or claim 45, wherein the solid tumor is selected from a sarcoma, a carcinoma, a lymphoma, a melanoma and a blastoma.

47. The method of any one of claims 1 to 46, wherein the cancer is selected from lung, bronchial, colorectal, prostate, pancreatic, liver, esophageal, urinary, bladder, kidney, renal, breast, ovarian and brain cancers, glioblastoma, and non-Hodgkin lymphomas.

48. The method of any one of claims 1 to 38, wherein the subject is previously untreated.

49. The method of any one of claims 1 to 48, further comprising a conventional cancer therapy.

50. The method of claim 49, wherein the conventional cancer therapy is surgery, radiotherapy, chemotherapy.

51. The method of any one of claims 1 to 50, wherein upon completing treatment with the VEGF-C antagonist and the anti-neoplastic composition, the subject receives further chemotherapeutic treatment with at least one chemotherapeutic agent.

52. The method of any one of claims 1 to 51, wherein the subject does not experience significant toxicity or adverse effect.

53. The method of any one of claims 1 to 52, wherein administering the VEGF-C antagonist and the anti-neoplastic composition to the subject effectively: produces tumor regression; decreases tumor weight or size; or increases time to progression, duration of survival, duration of progression-free survival, duration of response of the human subject, overall response rate in a group of human subjects, or quality of life.

54. A method for increasing the duration of survival of a human subject susceptible to or diagnosed as having a cancer, comprising administering to the subject in combination effective amounts of a VEGF-C antagonist and an anti-neoplastic composition.

55. A method for increasing the progression-free survival of a human subject susceptible to or diagnosed as having a cancer, comprising administering to the subject in combination effective amounts of a VEGF-C antagonist and an anti-neoplastic composition.

56. A method for treating a group of human subjects susceptible to or diagnosed as having a cancer, comprising administering to subjects in the group in combination effective amounts of a VEGF-C antagonist and an anti-neoplastic composition.

57. A method for increasing the duration of response in a human subject or a group of human subjects susceptible to or diagnosed as having a cancer, comprising administering to the subject or subjects in the group in combination effective amounts of a VEGF-C antagonist and an anti-neoplastic composition.

58. A method of treating a human subject or a group of human subjects having metastatic colorectal cancer, prostate cancer, pancreatic cancer or glioblastoma, comprising administering to the subject or subjects in the group in combination effective amounts of a VEGF-C antagonist and an anti-neoplastic composition, wherein the anti-neoplastic composition comprises at least one chemotherapeutic agent, wherein administration of the VEGF-C antagonist and the anti-neoplastic composition results in statistically significant and clinically meaningful improvement of the treated subject or group as measured by the duration of survival, progression free survival, response rate or duration of response.

59. A kit comprising a VEGF-C antagonist when used for treating cancer in a human subject, wherein the VEGF-C antagonist is for administering to the subject in combination with an anti-neoplastic composition.

60. The kit of claim 59, further comprising an anti-neoplastic composition.
